(19) United States
(12) Patent Application Publication
(10) Pub. No.: US 2023/0310300 A1
(43) Pub. Date: Oct. 5, 2023

(54) HAIR COLOURING COMPOSITIONS COMPRISING A BIO-BASED POLYMER AND A CROSSLINKER

(71) Applicant: HFC PRESTIGE SERVICE GERMINY GMBH, Darmstadt (DE)

(72) Inventors: Corinne Violette Mohr, Lorsch (DE); Graham Neil McKelvey, Glashütten (DE); Matija Crne, Wiesbaden (DE); Simon Paul Godfrey, Oberursel (DE); Mathias Kurt Herrlein, Kronberg (DE); Thomas Koch, Seeheim (DE); Judith Kaumanns, Mühltal (DE); Achim Weber, Altbach (DE); Melanie Dettling, Rottenburg (DE); Sarah Schmidt, Köngen (DE); Steffen Thomas Erler, Darmstadt (DE)

(21) Appl. No.: 18/012,577

(22) PCT Filed: Jun. 29, 2021
(86) PCT No.: PCT/EP2021/067924

§ 371 Date: Dec. 22, 2022

Related U.S. Application Data
(60) Provisional application No. 63/046,102, filed on Jun. 30, 2020.

(30) Foreign Application Priority Data
May 17, 2021 (EP) 21174134.3

(51) Int. Cl. A61K 8/65; A61Q 5/06; A61K 8/41; A61K 8/46; A61K 8/84

(52) U.S. Cl. CPC A61K 8/65; A61Q 5/065; A61K 8/41; A61K 8/46; A61K 8/84; A61K 2800/95; A61K 2800/884; A61K 2800/81

(57) ABSTRACT

The instant disclosure generally relates to a composition for treating or coloring keratin fibers, the composition comprising at least one bio-based polymer and optionally a crosslinker. The composition may also comprise pigment microparticles. The composition formed and set in situ as a solid linked coating has a substantially permanent pigment lastingness and minimally alters the keratin fibers.

### BACKGROUND

Treatments to mammalian or synthetic keratin fibers, and their surfaces, are well known in the art. Mammalian keratin fibers (natural hair) is structured as a cuticle or outer surface layer, a cortex which is an internal layer containing melanin or color bodies and keratin bundles, and sometimes a central core termed the medulla. Typical dye treatments focus on changes of the cortex. Another treatment focuses on addition of pigments to the surfaces of hair strands. Typical treatment for surface coloration attaches pigment particles with glue-like material. Of particular note for all of these treatments is their ability to alter the appearance of the hair, for example by changing the color or reflective properties of hair.

For dye treatments the alteration of appearance can be achieved through treating hair with a formulation containing dye molecules (so call direct dyes) which diffuse into or are absorbed on and sometimes through the cuticles of the hair fibers. Alternatively, so called oxidative dyes may be employed wherein the dye precursors diffuse into the hair and then react to form colored species within the hair including within the cortex of the hair. Often the oxidative dye products are designed to also lighten the hair, decolorizing some of the melanin within the cortex to enable a wider range of colors to be achieved. Over time the color imparted to the hair is removed during washing. This can happen rapidly for so called direct dyes and leads to a short-term change in hair appearance, typically lasting for a few washes. The so-called oxidative dyes may last considerably longer, and indeed removing the color can be hard to achieve, even after a considerable number of washes. When oxidative dyes are eventually removed by washing out, the melanin has also been decolorized by bleaching so that it will not return to its original color but to a lighter color. Unfortunately, the process of decolorizing hair leads not only to a lightening of the hair but also to a change in the perceived tone of the hair, leading to what is often described as an off tone or brassy result where the hair looks more orange than untreated hair of a similar lightness.

One disadvantage of the known oxidative dye technologies in this area is that the methods involve applying coloring compositions for an extended period of time to enable the color to develop within the hair. These compositions in some cases may also lead to some temporary scalp irritation. Together, the extended waiting time and potential irritation, prevents the hair coloration process for some users from being a pleasant or a so-called wellness experience. Such coloring compositions may also alter the hair structure itself, leading to oxidation of the hair surface, and partial degradation of the keratinous proteins from which the hair structure is constructed. With repeated coloring, these changes in hair structure become more pronounced and can be felt as poor hair condition. The color obtained when coloring with such a composition is also hard to predict, and even highly experienced users can still be surprised with the actual results that are obtained for a given product. Yet another drawback known to such coloring technologies is that, once the color is on and within the hair, the dye-based coloring material is difficult to remove and/or cannot be completely removed. Another drawback for the dye-based approach is that the application of hair coloration materials can yield uneven results as adherence to the surface and/or penetration of hair coloration materials into the hair can vary with hair type for example for a consumer differing color results may be visible between hair roots and hair tips. This can lead to an unnatural looking result. Some desired differences may still be visible due to the non-uniformity in coloration of the underlying hair, for example subtle difference in strand to strand levels of pheomelanin and eumelanin in a consumer may yield slightly different color results, even when the same color pigments or dyes are applied to a consumer. While some strand to strand variation is needed to provide natural looking hair, too much or too little can again lead to an unnatural looking color result. Due to the number of factors that determine the final hair color result for example, the length of application time, the underlying hair color, the hair changes from root to tip, it's hard for even experienced users to accurately predict the final color result and look.

As mentioned at the outset, alternative techniques have also been investigated. One such approach has involved coating hair strands with color pigment particles. The approach involves attaching the pigment particles to the hair strands using polymeric coatings which are able to hold the pigment particles by covalent bonds, ionic interactions, or entrapped within a matrix of the polymeric coating. Similarly, such polymeric coatings have been used as well without pigment particles, for providing some protection from environmental impacts, or simply for providing additional styling options.

If not removed actively by applying specific removal compositions, these polymeric coatings anyway will be gradually removed from the hair strands over the time inadvertently, for example by mechanical disruption such as combing, and in particular by washing the hair. When removed by washing, the polymeric coatings will end up in wastewater treatment plants, and finally in the oceans.

Up to now, the oligomers, polymers and other building blocks used for forming the crosslinked polymer coatings on hair strands have been derived from conventional materials, i.e. starting materials derived from fossil fuel resources. Fossil fuels are a limited resource, and efforts to replace materials based on fossil fuels by materials from renewable resources are a world-wide challenge. In addition, polymers synthetized from materials derived from fossil fuel resources often are not biodegradable, therefore attributing to environment pollution. Polymeric coatings from hair strands made from no-biodegradable materials and removed by washing will end up as microplastics in the oceans, and may finally find its way back into the food chain.

It is therefore an object to develop a clean, environment-friendly technology that uses renewable resources, does not result in harm to hair protein, is user friendly, provides appropriate color and luster, and leaves the hair manageable, free flowing and capable of moving naturally.

### SUMMARY

These and other objects are accomplished by aspects of the composition and method of use of the present invention. According to aspects of the invention, the composition, method and coated keratin fiber embodiments such as hair of any sort and other keratin fibers provide a clean, environment-friendly coating or coloration of keratin fibers. Especially for hair of all kinds, the coating or coloration that may be substantially uniform to significantly varied, may give hair strands an appearance of lower or higher chroma, shiny or reflective nature. The coloration aspects provide color fastness during a series of washes with shampoo or soap yet with appropriate formulations can be readily removed to leave the natural shade of the hair. These aspects significantly lessen and/or avoid treatment of hair that may cause breakage of keratin protein intermolecular bonds.

An aspect of the invention concerning the composition provides embodiments wherein at least one bio-based polymer is dissolved in an aqueous medium. Being dissolved in an aqueous medium implies that the at least one bio-based polymer has a minimum solubility in the aqueous medium as defined herein. The at least one bio-based polymer further comprises functional groups, also referred to herein as “first” functional groups.

The first functional group of the bio-based polymer may be reactive with another, identical first functional group (hereinafter a self-reactive functional group). The other self-reactive functional group may be present on the same molecule of the at least one bio-based polymer (intra-chain crosslinking), on a different molecule of the at least one bio-based polymer (inter-chain crosslinking), on a crosslinker, or on a keratin fiber. Examples of pairs of self-reactive functional groups are, for instance (mercapto and mercapto), and (olefinoyl and olefinoyl).

Alternatively, the first functional groups of the at least one bio-based polymer forms one part of a pair of complementary functional groups. The remaining part of the pair of complementary functional groups may be present on the same molecule of the at least one bio-based polymer (intra-chain crosslinking), on a different molecule of the at least one bio-based polymer or a different bio-based polymer (inter-chain crosslinking), on a crosslinker, or on a keratin fiber. The complementary pairs may be designated as first and second functional groups. The complementary reactive pairs and the self-reactive functional group can be reactively combined in situ to covalently bond together. Because the complementary reactive pairs and self-reactive functional groups are parts of large molecules having dipolar groups, hydrogen bonding groups and large lipophilic groups, the in situ interaction may also involve electrostatic, ionic, hydrogen bond, coordinate or entanglement interaction. Embodiments comprising the at least one bio-based polymer and a crosslinker with different first and second functional groups typically are kept separate until application to keratin fibers.

The composition comprises at least one bio-based polymer. Bio-based polymers generally encompass synthetic polymers produced from renewable resources, synthetic polymers corresponding to polymers produced in a cell of a living organisms (irrespective of the origin of the material of the polymer), biodegradable polymers (irrespective of the origin of the material of the polymer), and biopolymers. Preferred bio-based polymers are biodegradable. Particularly preferred bio-based polymers are bio-degradable polymers derived from renewable resources; polymers derived from renewable resources and corresponding to polymers produced in a cell of a living organism; and biopolymers.

Further, the composition may comprise a mixture of two or more bio-based polymers. Mixtures of bio-based polymers may comprise different bio-based polymers of the same type, for example proteins having different amino acid sequences, such as gelatin and albumin. Alternatively, mixtures of bio-based polymers may comprise bio-based polymers of different types, for example protein and polysaccharide. Another example of bio-based polymers of different types is carboxy-functional polysaccharides and amino-functional polysaccharides.

In embodiments, the composition comprises polymers other than bio-based polymers. Considering the environmental aspect of the present invention, however, the presence of polymers other than bio-based polymers is less preferred. According to embodiments, at least 50% by weight of the polymers present in the composition, based on the total dry weight of all polymers present in the composition, are bio-based polymers. According to particular embodiments, less than 20% by weight or even less than 10% by weight, based on the sum of the total dry weight of bio-based polymers and polymers other than bio-based polymers, are polymers other than bio-based polymers.

According to embodiments, the at least one bio-based polymer is a biopolymer, i.e. a polymer produced in (and obtained from) a cell of a living organism, which polymer has been modified by derivatization. Derivatization of such polymers may encompass modification on basis of monomer structure, and modification of polymer structure. Modification of the monomer structure includes masking of functional groups or de-masking of groups, activation of functional groups, or modification of functional groups in order to change the charge density, to change the solubility or to change the rheology (viscosity). According to embodiments, the biopolymer may comprise two or more types of monomer structure modifications. Modification of the monomer structure is feasible in an amount of up to about 80% of the repeating units or monomers, respectively. Typically, not more than 40% of the repeating units or monomers, respectively, are modified by masking, activation, changing the charge density, changing the solubility or changing the rheology (viscosity). Modification of the polymer structure includes, for example, decreasing the molecular weight of polymer chains, changing the crosslink density, or changing the secondary structure of biopolymers.

Embodiments of the composition also may include a crosslinker. Crosslinkers catalyze or initiate the formation of covalent bonds, hydrogen bonding, electrostatic or ionic interaction, ionic gelation, etc. According to embodiments, the crosslinkers or remnants thereof become integrated into the crosslinked structure comprising the at least one bio-based polymer, or remain electrostatically or ionically associated with the at least one bio-based polymer. According to other embodiments, the crosslinker initiates the crosslinking, but remnants of the crosslinker are not discernible in the crosslinked polymer structure.

In embodiments comprising a crosslinker, the at least one bio-based polymer and the crosslinker usually are maintained separately, and mixed only shortly prior to application to the keratin fibers. Accordingly, during storage, the at least one bio-based polymer typically is maintained in a first compartment and the crosslinker is maintained in a second compartment.

Embodiments of the composition for coloring keratin fibers may also comprise pigment particles (also synonymously described herein as pigment microparticles). The pigment particles may comprise irregular shapes of at least one pigment color and have at least one dimension of less than one micron.

Embodiments of the pigment microparticles used on the composition described herein may comprise organic pigment microparticles, which imparts color to the hair, having a given D50[vol], and pigment microparticles, for providing light scattering properties to the colored hair, having a D50[vol] which is larger than the D50[vol] value of the organic pigment microparticles. Embodiments may also include microparticle metal flakes for light reflection to add shine to the desired color or to make the hair appear to be lighter than the starting hair color.

Embodiments of the method for applying the composition to keratin fibers focus on the self-reactive features of the at least one bio-based polymer, or on the combination of reactive features of the at least one bio-based polymer, or on the combination of reactive features of the at least one bio-based polymer and the crosslinker. For embodiments of the method utilizing crosslinker, the composition and the crosslinker may be mixed together before application to the keratin fiber, may be applied separately and simultaneously to the keratin fiber, or may be applied sequentially to the keratin fiber. Prior to the application of the composition to the keratin fiber, a pretreatment composition may be applied to the keratin fiber. According to embodiments, the pretreatment composition comprises a cationic polymer.

In addition to the at least one bio-based polymer, the optional crosslinker and the optional pigment microparticles, the composition may optionally contain additional ingredients helpful and beneficial to the keratin fiber and/or its coloration. These additional ingredients include but are not limited to one or more of dispersants, surface treatment agents for the pigment microparticles, plasticizers, conditioners, suspending agents, thickening agents, adjuvants, moisturizers, surfactants, fatty substances, waxes, fatty amides and soluble organic dyes of colors different from those of the pigment microparticles.

An aspect of the invention concerning the wash-fastness or remanence of the coating on the keratin fiber, and especially on hair strands, comprises the ability of the coating to resist dissolution by ordinary cleaning of the keratin fibers such as hair. Ordinary cleaning may involve washing with soap and water, washing with an aqueous dilution of shampoo and washing with water.

An additional aspect of the invention concerns the application of the composition to keratin fiber such as brows, lashes and skin as well as to hair on the scalp. Additionally, the composition may be applied to animal hair or fur. The composition may be applied to these kinds of keratin fibers with appropriate adjustments of the composition parameters within the parameters described for hair on the scalp. Typically, the eyebrow hair may be treated with the composition using parameters similar to or the same as those of the composition for hair on the scalp. The hair of eyelashes typically can be similarly treated with the composition for eyebrows and the viscosity adjusted to provide a somewhat more viscous composition for application to the eye lashes. For skin, the parameters of the composition may have a higher solids content and viscosity may be adjusted to provide embodiments that will not readily drip or otherwise flow off the skin surface to which the composition is applied. The composition for skin will preferably have higher in situ linking to provide a durable coating or covering on the keratin skin substrate.

According to a particular aspect, the composition of the present invention is for coloring a keratin fiber. According to embodiments, a composition for coloring a keratin fiber comprises an aqueous medium, at least one bio-based polymer selected from proteins and polysaccharides, wherein the at least one bio-based polymer comprises olefinoyl-functional groups and is dissolved in the aqueous medium, at least one crosslinker comprising amine-functional groups and/or mercapto-functional groups, and one or more pigments dispersed in the aqueous medium.

Yet another aspect of the invention directed to achievement of the color coating on keratin fibers, especially on anagenic hair, concerns embodiments of the priming and/or deep cleaning of the surfaces of the keratin fibers. These embodiments are developed through practice of Praeparatur and Fundamenta techniques. These techniques deal with unique issues of anagenic hair such as but not limited to sebum coating on the keratin fiber strand surfaces, bound fatty acid layer (F layer) attached to keratin fiber strand surfaces, grime, grit, foulness and deposits from hair formulations previously applied to anagenic hair. The Praeparatur technique substantially to essentially primes the hair to remove surface crusting, sebum and/or glazing while the Fundamenta technique deep cleans the surface character and/or surface structure of keratin fibers as well as removes the F layer. These techniques may be applied separately and individually or may be applied together in either sequence. Irrespective of use of both or use of one or the other, these techniques are applied to hair before applying the pretreatment and film forming compositions. It is believed that the combination of one or more of the Praeparatur/Fundamenta techniques coupled with the pretreatment small molecule forming a network intimately adhering to the contours of the treated topographic surfaces of keratin fibers and the adherence among and between the small molecule network and the film forming composition produce a highly remanent color coating on anagenic hair.

Embodiments of the Praeparatur technique include but are not limited to mild agitation with an aqueous surfactant composition to strong interaction with an aqueous or aqueous organic medium with anionic surfactant and/or rinsing with aqueous media optionally having pH adjustment. Additional procedures include optional mechanical agitation with such surfactant media and combing, brushing, vibrating, ultrasound and similar vibratory action applied to the surfaces of keratin fibers.

Embodiments of the Fundamenta method involve restructuring of the hair strand surfaces including F layer removal and include but are not limited to one or more of a non-thermal equilibrium plasma treatment; chemical treatment with a phase transfer tenside such as a multi-alkyl ammonium halide; chemical treatment with an oxidative agent such as persulfate, ozone or a peroxide such as benzoyl peroxide or hydrogen peroxide with optional alkali and optional surfactant cleaning.

Aspects of the invention are directed to embodiments of methods for application of the pretreatment composition, the film forming composition, and Praeparatur and Fundamenta techniques to form a color coating on keratin fibers such as anagenic hair. Embodiments of the methods comprise parameters, conditions and techniques for any one or more of: a) an application of the pretreatment composition to keratin fibers, preferably anagenic hair; b) an application of the film forming composition to keratin fibers, preferably anagenic hair; c) Praeparatur techniques applied to keratin fibers, preferably anagenic hair; and d) Fundamenta techniques applied to keratin fibers, preferably anagenic hair or any combination of these methods to form a color coating on keratin fibers, preferably anagenic hair.

The methods of the invention involving parameters, conditions and techniques for application of film forming composition may be directed to application of the film forming composition alone to keratin fibers. However, it is preferred that the methods of the invention involve parameters, conditions and techniques for application of the pretreatment composition to keratin fibers, preferably anagenic hair before, or simultaneous with, or mixed with, or in combination with the application of film forming composition. Additionally, application of sequential and/or simultaneous and/or mixed combinations of the pretreatment composition and the film forming composition may call for prior application of Praeparatur and/or Fundamenta techniques to keratin fibers, preferably anagenic hair.

According to the invention, the desirable characteristics of the color coatings on keratin fibers, preferably anagenic hair, may be demonstrated by tests of the coloration on hair tresses prepared from unbleached natural white human hair (hereinafter untreated hair tresses), bleached natural white human hair (hereinafter treated hair tresses) and untreated hair tresses specially prepared with sebum so as to mimic anagenic hair (hereinafter mimic hair tresses). The two base line forms of hair tresses (untreated and treated hair tresses) are not anagenic hair in that they are cut natural hair so that they are not bathed in sebum secretion but nevertheless have the F layer coating. The untreated hair tresses are substantially hydrophobic, have low porosity and have little or no keratin protein surface disruption. The treated hair tresses are substantially less hydrophobic to more hydrophilic than, and have greater porosity than, the untreated hair tresses. The treated hair tresses also display low to mild keratin protein surface disruption. The mimic hair tresses are especially treated to demonstrate the sebum, F layer, grubbiness, foulness, and root, mid-length and tip issues associated with anagenic hair and especially those associated with the root segment of anagenic hair. The root segment of anagenic hair is constantly bathed in sebum and has the F layer coating so that the root segment of anagenic hair shows the most extreme behavior difference relative to treated and untreated hair tresses. To establish the mimicry, the untreated hair tresses undergo a series of preparations designed to establish a close comparison with the behavior of anagenic hair, especially the root segments. To this end, embodiments of color coatings according to the invention have been studied with anagenic hair, untreated hair tresses, treated hair tresses. The results of these preliminary studies have established the techniques and components for development of these hair tresses to enable their similarity to the behavior of anagenic hair, especially the root segments of anagenic hair. Untreated, treated, and mimic hair tresses are accordingly able to function as the substrates for experimental development of the embodiments of the color coatings and methods for hair coloration with anagenic hair.

When color coatings are formed onto untreated and onto treated tresses, long lasting remanence is exhibited. However, when the same color coatings are applied to anagenic hair such as the hair of a live salon hair model, remanence disappears. For efficiency of experimental practice, the above described mimic hair tress has been developed to come as close as possible to the behavior of anagenic hair, especially the root segments of anagenic hair.

### BRIEF DESCRIPTION OF DRAWINGS

FIG. 1 schematically shows biopolymers and derivatives of biopolymers:

FIG. 1a schematically shows sodium alginate

FIG. 1b schematically shows chitosan

FIG. 2 schematically shows polymer modifications:

FIG. 2a schematically shows gelatin modified by activation of an amino-functional group with methacrylic anhydride to give a methacryloyl-functional group

FIG. 2b schematically shows activation of an amino-functional group of gelatin with acrylic anhydride to give gelatin having an acryloyl-functional group

FIG. 2c schematically shows activation of an amino-functional group of gelatin with glycidyl acrylate to give gelatin having an acryloyl-functional group

FIG. 2d schematically shows gelatin modified by masking of an amino-functional group with acetic anhydride to give a masked acetylated group

FIG. 2e schematically shows gelatin modified by reacting a carboxy-functional group with ethylene diamine to give a positively charged (cationized) group at moderately acidic pH.

FIG. 3 shows covalent crosslinking of a mixture of olefinoyloxy-functional and olefinoylamino-functional groups, for example by UV in the presence of a compound initializing free-radical crosslinking, such as a photoinitiator.

FIG. 4 shows covalent crosslinking of amino-functional groups and carboxy-functional groups by EDC. EDC mediates the formation of the carboxy amide crosslinks but does not become integrated into the crosslinked polymer structure.

FIG. 5 shows ionic crosslinking by ionic gelation of amino-functional groups of chitosan with polyphosphate.

FIG. 6 shows covalent crosslinking of an olefinoylamino-functional group with an amine-functional crosslinker such as PEI by Michael Addition.

FIG. 7 schematically shows the impact of acetyl-masking groups on the rheological properties of modified gelatins GM and GMA. GM2 has some of the functional groups modified to crosslinkable methacryloyl groups (shown in green) but still has the ability to gel (shown as lines between NH/OH groups). GM10 has more of those crosslinkable groups and therefore less NH/OH groups left to gel. GM2A8 has the same amount of crosslinkable groups as GM2, but the acetylic groups mask most of the residual NH/OH groups so that GMA has less ability to gel.

FIG. 8 shows experimental results of hair coloring, and wash-fastness:

FIG. 8a shows hair coloring results obtained with a composition comprising chitosan as the at least one bio-based polymer, polyisocyanate as a crosslinker, and pigment.

FIG. 8b shows hair coloring results obtained with a composition comprising methacryloylated gelatin as the at least one bio-based polymer, polythiol as a crosslinker, and pigment.

FIG. 8c shows hair coloring results obtained with a composition comprising acryloylated gelatin as the at least one bio-based polymer, PEI as a crosslinker, and pigment.

FIG. 8d shows hair coloring results obtained with a composition comprising cationized methacryloylated gelatin as the at least one bio-based polymer, EDC as a crosslinker, and pigment.

FIG. 9 shows LC-MS chromatograms of samples of biopolymers described herein, samples of crosslinked biopolymers described herein, and the LC-MS chromatograms of these samples after biodegradation by Proteinase K.

FIG. 10 shows UV detection at 205 nm of samples shown in FIG. 9, and references.

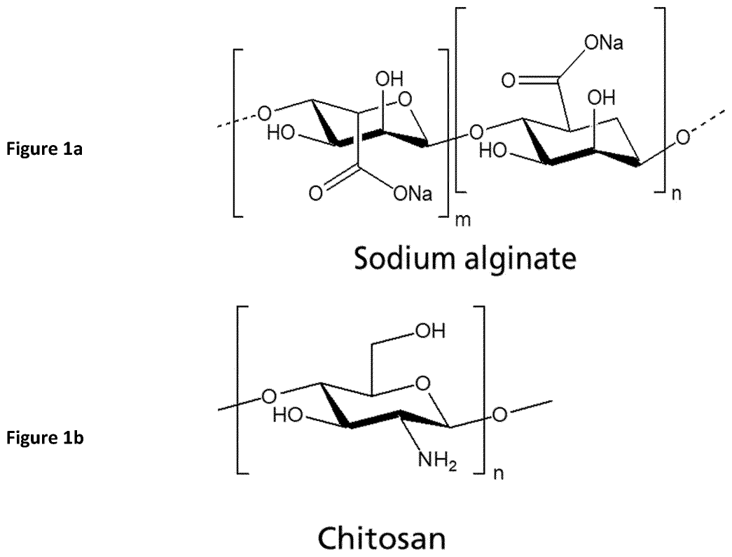

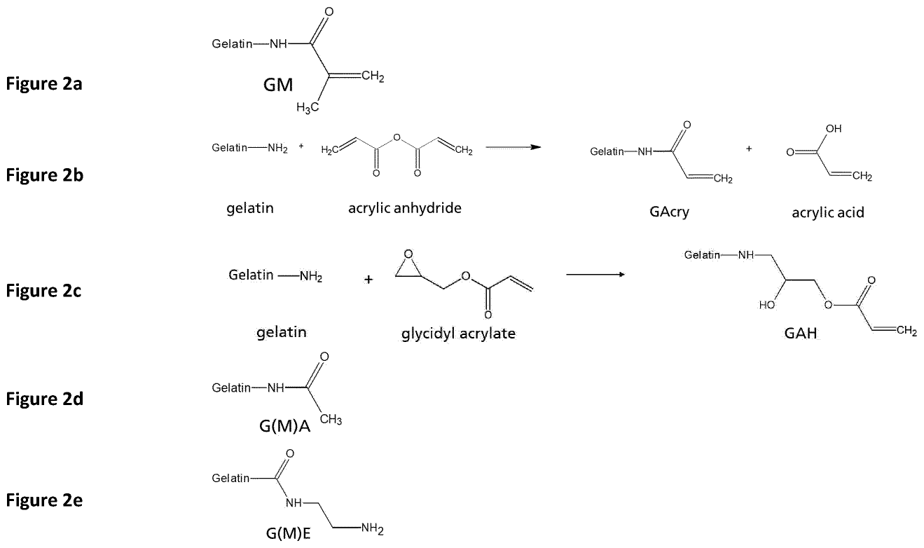

### DEFINITIONS

Unless defined otherwise, all technical and scientific terms used herein have the same meaning as commonly understood by a person of ordinary skill in the art.

As used in the specification and the appended claims, the singular forms “a,” “an” and “the” include plural referents unless the context clearly dictates otherwise

The term “may” in the context of this application means “is permitted to” or “is able to” and is a synonym for the term “can.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B.

The molecular weight of a polymer or oligomer used according to the invention may be measured by a weight average molecular weight, and the distribution of molecules of different molecular weights of a polymer or oligomer used according to the invention is determined by its polydispersity index. Molecular weight is expressed herein in g/mol. Other units for the molecular weight used in the technical field are daltons (Da), kiloDaltons (KDa) and megaDaltons, which is million daltons or (MDa). One Da is equal to one g/mol. The acronym M_{w} stands for weight average molecular weight, Mₙ is the number average molecular weight of a given polymer. Polydispersity is a unit-less number and indicates the breadth of the distribution of the polymer molecular weights and is defined as the M_{w}/Mₙ. Weight average molecular weight, number average molecular weight, and polydispersity can be determined using size exclusion chromatography in accordance with international accepted standard methods. Different standard methods may be applicable for different types of polymers.

Molecular weight parameters of neutral polymers and polyanions are determined according to ISO/DIS 13885-3, while the molecular weight parameters of chitosans are determined according to ASTM F2602-18. Molecular weight parameters of polymers not covered by the above methods may be determined according to ISO 16014-2, ISO 16014-3, ISO 16014-4 or ISO 16014-5. Molecular weight parameters of polymers not covered by the above methods preferably are calculated from a calibration curve constructed using polymer standards, for example as described in ISO 16014-2.

The term “about” is understood to mean±10 percent of the recited number, numbers or range of numbers.

The term “about 0 wt %” is understood to mean that no substance, compound or material to which zero (0) refers is present, up to a negligible but detectable amount is present, assuming that the detectability can be determined on a parts per million basis.

In addition, where features or aspects of the invention are described in terms of Markush groups, those skilled in the art will recognize that the invention is also thereby described in terms of any individual member or subgroup of members of the Markush group. For example, if X is described as selected from the group consisting of methyl, ethyl or propyl, claims for X being methyl and claims for X being methyl and ethyl are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The term “keratin fibers” as used herein means any natural or synthetic fiber made of or comprising a keratin. In particular, the term “keratin fibers” encompasses natural or synthetic keratin fibers. Preferred keratin fibers are natural keratin fibers. Natural keratin fibers include those from mammals and/or on mammals including human, primate, ruminant, camelid, equine, rodent and neovison including but not limited to cow, sheep, deer, goat, buffalo, lama, alpaca, camel, guanaco, vicuna, horse, antelope, moose, elk, rat, mouse, beaver, rabbit, mink, monkey, ape and similar species. Natural keratin fibers may include hair or fur. Particularly preferred keratin fibers in the present invention are mammalian keratin fibers. The term “keratin fibers” is used interchangeably in this document with the terms “hair” or “hair strands” unless the context dictates otherwise.

As used herein “anagenic hair” means hair strands that are in direct connection with a hair follicle which is in either the anagen or telogen state. Anagenic hair is present in one of these states on a scalp of a person, a human. The follicle of anagenic hair produces long chain fatty acids, so-called F-layer, which form a water-resistant coating on the cuticle of the hair shaft. Joining the hair follicle channel is a sebaceous gland that secretes sebum onto the hair shaft and onto the scalp. As a strand of hair grows from the follicle and extends from the scalp, sebum produced at the follicle spreads out from the follicle and continues to coat the strand. Sebum is removed at least in part from strand ends by shampooing but is replenished by this continued production. Hair cut from a living person is no longer anagenic hair.

As used herein, the terms “covalent, coordinate, electrostatic, ionic, dipolar and entanglement or entwining interactions” mean a chemical relationship between two atoms or two groups of atoms. The interaction includes a covalent bond between the atoms such as the covalent bond between the two carbons of ethane. The interaction includes a coordinate bond between two or more atoms such as the coordinate bond between oxygen and sulfur of the sulfate anion (SO₄⁻²) or a complex of zinc and EDTA. The interaction includes an electrostatic or ionic interaction between two charged atoms or particles such as the interaction between sodium and chloride of salt or between ammonium and acetate of ammonium acetate. Dipolar interaction includes hydrogen bonding such as the interaction between water and the hydroxyl of methyl alcohol. The interaction includes entanglement or entwining which is lipophilic interaction or mechanical/physical twisting together such as is present in the molecules of polyethylene.

As used herein, the term “transfer resistance” generally refers to the quality exhibited by compositions that are not readily removed by contact with another material, such as, for example, an item of clothing or the skin. Transfer resistance can be evaluated by any method known in the art for evaluating such transfer. For example, transfer resistance of a composition can be evaluated by the amount of product transferred from a wearer to any other substrate after the expiration of a certain amount of time following application of the composition to the hair. The amount of composition transferred to the substrate can then be evaluated and compared. For example, a composition can be transfer resistant if a majority of the product is left on the wearer's hair. Preferably little or no composition is transferred to the substrate from the hair.

As used herein, the term “minimally alters the keratin material or fibers, upon application” generally means that after removal of the composition coating on the keratin fibers such as hair, the keratin fibers are returned to a substantially unaltered state. The state of the keratin fibers such as hair can be assessed for example using ATR FT-IR for oxidative damage as described later or through tensile testing methods known to those skilled in the art for assessing fiber strength for example using equipment such as those designed and sold by Dia-Stron™.

As used herein, the term “setting” means converting the composition to a solid coating through the application of means designed to remove or otherwise separate the medium from the other constituents of the composition so as to leave a solid coating of the bio-based polymer and other optional ingredients of the composition.

“Aliphatic substituent, group or component” refers to any organic group that is non-aromatic. Included are acyclic and cyclic organic compounds composed of carbon, hydrogen and optionally of oxygen, nitrogen, sulfur and other heteroatoms. This term encompasses all of the following organic groups except the following defined aromatic and heteroaromatic groups. Examples of such groups include but are not limited to alkyl, alkenyl, alkynyl, corresponding groups with heteroatoms, cyclic analogs, heterocyclic analogs, branched and linear versions and such groups optionally substituted with functional groups, as these groups and others meeting this definition of “aliphatic” are defined below.

“Aromatic substituent, group or component” refers to any and all aromatic groups including but not limited to aryl, aralkyl, heteroalkylaryl, heteroalkylheteroaryl and heteroaryl groups. The term “aromatic” is general in that it encompasses all compounds containing aryl groups optionally substituted with functional groups (all carbon aromatic groups) and all compounds containing heteroaryl groups optionally substituted with functional groups (carbon-heteroatom aromatic groups), as these groups and others meeting this definition of “aromatic” are defined below.

As used herein, the term “optionally” means that the corresponding substituent or thing may or may not be present. It includes both possibilities.

“Alkyl” refers to a straight or branched or cyclic hydrocarbon chain group consisting solely of carbon and hydrogen atoms, unless otherwise specifically described as having additional heteroatoms or heterogroups. The alkyl group contains no unsaturation, having from one to twenty four carbon atoms (e.g., C₁-C₂₄ alkyl). Whenever it appears herein, a numerical range such as for example but not limited to “1 to 24” refers to each integer in the given range; e.g., “1 to 24 carbon atoms” means that the alkyl group may consist of 1 carbon atom, 2 carbon atoms, 3 carbon atoms, etc., up to and including 24 carbon atoms, although the present definition also covers the occurrence of the term “alkyl” where no numerical range is designated. In some embodiments, it is a C₁-C₄ alkyl group. In other instances it is a C₁-C₆ alkyl group and in still other instances it is a C₁-C₂₄ alkyl group Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like.

“Alkylenyl” refers to a straight or branched divalent hydrocarbon chain consisting solely of carbon and hydrogen atoms, unless otherwise specifically described as having additional heteroatoms or heterogroups. The alkylenyl group contains no unsaturation has a valence bond at either end of the chain and has a numerical range of carbon atoms of 1 to 24, which numerical range includes each integen in the range. An example of a divalent hydrocarbon chain designated as an alkylenyl group is —CH₂—CH₂—CH₂—CH₂— which is butylenyl.

“Cycloalkyl” is a subcategory of “alkyl” and refers to a monocyclic or polycyclic group that contains only carbon and hydrogen, and may be saturated, or partially unsaturated. Cycloalkyl groups include groups having from 3 to 24 ring atoms (i.e., C₃-C₂₄ cycloalkyl). Whenever it appears herein, a numerical range such as but not limited to “3 to 24” refers to each integer in the given range; e.g., “3 to 24 carbon atoms” means that the cycloalkyl group may consist of 3 carbon atoms, etc., up to and including 24 carbon atoms. In some embodiments, it is a C₃-C₈ cycloalkyl group. In some embodiments, it is a C₃-C₅ cycloalkyl group. Illustrative examples of cycloalkyl groups include but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

“Alkoxy” refers to the group —O-alkyl, including from 1 to 24 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl is an alkyl group which encompasses both straight and branched chain alkyls of from 1 to 4 carbon atoms.

“Amino” or “amine” refers to an —N(R^{a})₂ group, where each R^{a} is independently hydrogen or linear, branched or cyclic alkyl of 1 to 6 carbons. When an —N(R^{a})₂ group has two R^{a} groups other than hydrogen, they can be combined with the nitrogen atom to form a 4-, 5-, 6-, or 7-membered ring.

“Aryl” refers to a conjugated pi group with six or ten ring atoms which has at least one ring having a conjugated pi electron system which is carbocyclic (e.g., phenyl, fluorenyl, and naphthyl). Bivalent groups formed from substituted benzene derivatives and having the free valences at ring atoms are named as substituted phenylene groups. Bivalent groups derived from univalent polycyclic hydrocarbon groups whose names end in “-yl” by removal of one hydrogen atom from the carbon atom with the free valence are named by adding “-idene” to the name of the corresponding univalent group, e.g., a naphthyl group with two points of attachment is termed naphthylidene. The term includes monocyclic or monocyclic-ring polycyclic (i.e., rings which share adjacent pairs of ring atoms) groups.

“Heteroalkyl” “heteroalkenyl” and “heteroalkynyl” include optionally substituted alkyl, alkenyl and alkynyl groups and which have one or more skeletal chain atoms selected from an atom other than carbon, e.g., oxygen, nitrogen, sulfur, phosphorus or combinations thereof. A numerical range may be given, e.g. C₁-C₂₄ heteroalkyl which refers to the chain length in total, which in this example may be as long as 24 atoms long. For example, a —CH₂OCH₂CH₃ group is referred to as a “C₄” heteroalkyl, which includes the heteroatom center in the atom chain length description. Connection to the rest of the molecule may be through either a heteroatom or a carbon in the heteroalkyl chain.

“Heteroaryl” or heteroaromatic refers to a 5, 6 or 10-membered aromatic group (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range refers to each integer in the given range. An N-containing “heteroaromatic” or “heteroaryl” moiety refers to an aromatic group in which at least one of the skeletal atoms of the ring is a nitrogen atom. The polycyclic heteroaryl group may be monocyclic or non-monocyclic. The heteroatom(s) in the heteroaryl group is optionally oxidized. One or more nitrogen atoms, if present, are optionally quaternized. The heteroaryl is attached to the rest of the molecule through any atom of the ring(s). Examples of heteroaryls include, but are not limited to adeninyl, azabenzimidazolyl, azaindolyl, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d] pyrimidinyl, 5,6,7,8,9,10-hexahydrocycloocta[d] pyridazinyl, 5,6,7,8,9,10-hexahydrocycloocta[d]pyridinyl,isothiazolyl, imidazolyl, imidazopyridinyl, isoxazolopyridinyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h] quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thianaphthalenyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl), xanthinyl, guaninyl, quinoxalinyl, and quinazolinyl groups.

“Heterocyclic” refers to any monocyclic or polycyclic moiety comprising at least one heteroatom selected from nitrogen, oxygen and sulfur. As used herein, heterocyclyl moieties can be aromatic or nonaromatic. The moieties heteroaryl and heterocyclyl alkyl are members of the heterocyclic group.

The term “olefinoyl” denotes a functional group of the formula —C(O)—CR1=CR2R3. The olefinoyl group is described herein in particular as an activated functional group susceptible to Michael reactions such as aza-Michael reactions, thio-Michael reactions or oxa-Michael reactions. In the above formula, R1, R2 and R3 may be H or optionally substituted alkyl. According to some embodiments, R1, R2 and R3 may be H or C1-C6 alkyl. According to preferred embodiments, R1 and R2 are selected from H and methyl, and R3 is H or C1-C6 alkyl. For example, R1 may be selected from H and methyl, while R2 and R3 both are H. Linkage of the olefinoyl group to a polymer may be via any of the usual backbone atoms, including C, O, N and S. As described herein, an olefinoyl group may be generated by reacting amine-functional groups or thio-functional groups or hydroxy-functional groups for example with an anhydride derived from unsaturated carboxylic acids. For example, reacting amine-functional groups or thio-functional groups or hydroxy-functional groups of the polymer(s) with (meth)acrylic anhydride gives (meth)acryloyl groups. Depending on the linkage of the olefinoyl group to a polymer, the present disclosure optionally further distinguishes the olefinoyl groups in terms of:
olefinoyloxy groups —O—C(O)—CR1=CR2R3 (by reaction with hydroxy)
olefinoylamino groups —NH—C(O)—CR1=CR2R3 (by reaction with amine)
olefinoylthio groups —S—C(O)—CR1=CR2R3 (by reaction with thio)

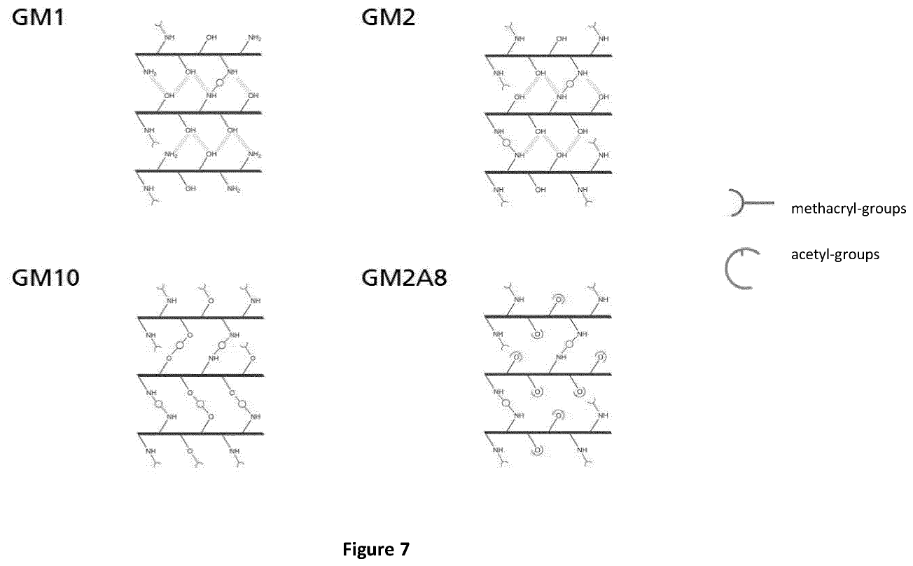

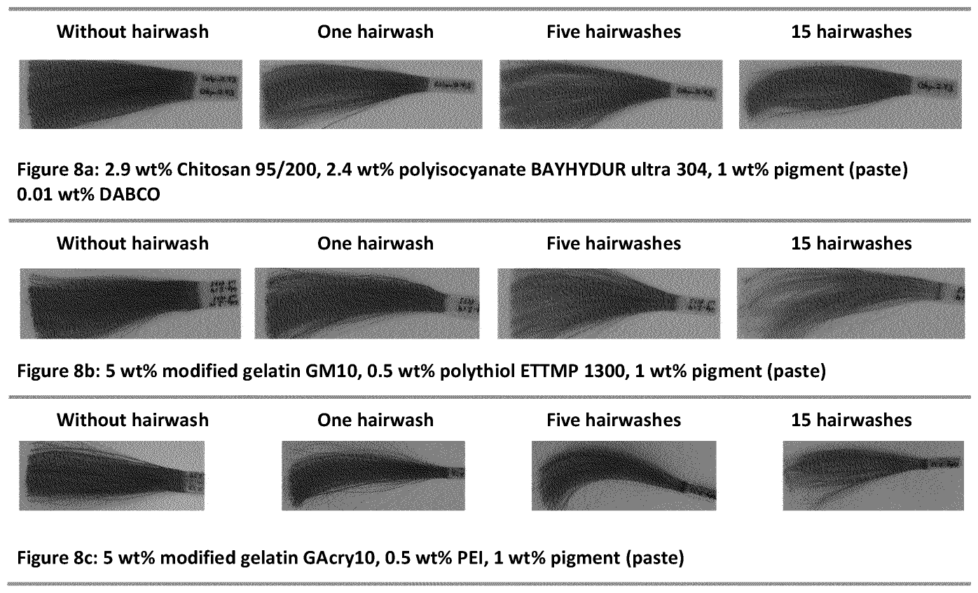

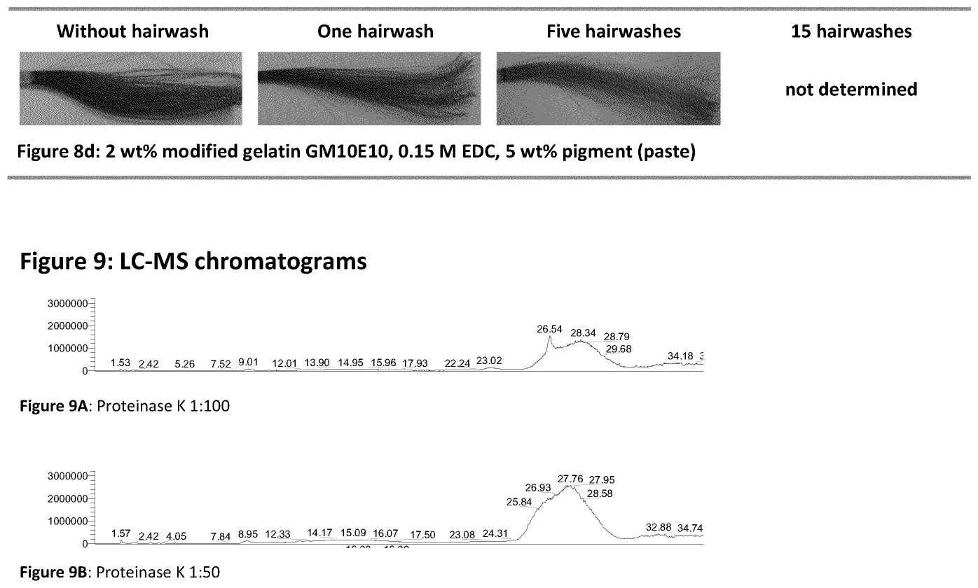

When a polymer comprising different functional groups is reacted to produce olefinoyl groups, the product obtained may comprise olefinoyl groups linked to the polymer via different bridging atoms or groups. For example, activating the functional groups of a protein to generate olefinoyl groups typically results in a protein having olefinoyloxy groups, olefinoylamino groups and maybe olefinoylthio groups. As shown for example in FIG. 7, reacting gelatin with (meth)acrylic anhydride initially will provide predominantly (meth)acryloylamino groups by activation of amine-functional groups (GM1, GM2). At higher activation levels, reaction of hydroxy-functional groups increasingly will contribute to the formation of (meth)acryloyloxy groups (GM10).

The terms “in situ-linkable” and “crosslinkable” mean the potential at a future time to form covalent bonds, coordinate linkages, ionic linkages, electrostatic linkages, polar couplings, hydrogen bonds and polymer entanglement to provide interactions and/or connections between molecules. The terms “in situ-linked” and “crosslinked” mean that in the present state, covalent bonds, coordinate linkages, ionic linkages, electrostatic linkages, polar couplings, hydrogen bonds and entanglement arrangements have already occurred.

“in situ” is a latin phase meaning in its original place. In the context of this invention, it means an activity such a crosslinking that takes place on the hair.

Zeta potential relating to pigment microparticles means the electrokinetic potential of extremely small particles suspended in colloidal dispersions. It is caused by the net electrical charge at the particle interface with the suspending fluid. It is an indicator of the stability of a colloidal dispersion. The magnitude indicates the degree of electrostatic repulsion between adjacent similar charged particles in a dispersion. At zero or minimal + or − potential, rapid coagulation can occur. At a + or − zeta potential above about 40, good colloidal stability is maintained. Zeta potential can be measured using approaches known to those skilled in the art. For example, a Zetasizer Nano Z from Malvern Panalytical may be used to assess the zeta potential of the components.

Biodegradability denotes the capability of a macromolecule to degradation or breakdown by microorganisms and/or abiotic factors such as humidity, heat or sunlight. Preferably, the bio-based polymers used in the present invention are biodegradable to monomers metabolizable by microorganisms, or to the biodegradation end-products methane and/or carbon dioxide and water. Even more preferably, also crosslinked polymer networks formed by bio-based polymers and optional crosslinkers are biodegradable to monomers and/or biodegradation end-products mentioned above.

Biodegradability is determined herein according to one of the following international test standards:
OECD 302C (% Inherent Biodegradability: Modified MITI Test (II) (OECD 302C), with pass criteria of ≥70% mineralisation within 14 days (test duration 28 days according to 302C), pre-adaptation of the inoculum not being allowed.
OECD 301B, C, D, F, fulfilling the Technical Guideline specific criteria for biodegradability in aquatic compartments, with pass criteria: ≥60% mineralisation in 28 days but 10-day window does not apply.
Enhanced/modified ready biodegradation: OECD TG 301 B, C, D, F extended to up to 60 days; means 60% mineralization after 60 days (10-day-window does not apply).

The following briefly describes the test standards noted above:

### OECD Test 302C: Inherent Biodegradability: Modified MITI Test (II):

This Test Guideline describes the modified MITI test (II). This test permits the measurement of the Biochemical Oxygen Demand (BOD) and the analysis of residual chemicals in order to evaluate the inherent biodegradability of chemical substances which have been found by the Standard MITI Method (I) to be low degradable.

An automated closed-system oxygen consumption measuring apparatus (BOD-meter) is used. Chemicals to be tested are inoculated in the testing vessels (six bottles with different quantities of test chemical) with micro-organisms. In order to check the activity of the inoculum, the use of control substances (aniline, sodium acetate or sodium benzoate) is desirable. During the test period, the BOD is measured continuously. Biodegradability is calculated on the basis of BOD and supplemental chemical analysis, such as measurement of the dissolved organic carbon concentration, concentration of residual chemicals, etc. The BOD curve is obtained continuously and automatically for 14 to 28 days. After the 14 to 28 days of testing, pH, residual chemicals and intermediates in the testing vessels are analyzed.

### OECD Test 301—Ready Biodegradability:

In this Guideline six methods are described that permit the screening of chemicals for ready biodegradability in an aerobic aqueous medium. For polymers relevant are:
301 B: CO2 Evolution (Modified Sturm Test)
301 C: MITI (I) (Ministry of International Trade and Industry, Japan)
301 D: Closed Bottle
301 F: Manometric Respirometry:

Methods 301 B, 301 D are modified versions of the earlier OECD Guidelines adopted in 1981.

Method 301 C is virtually identical with earlier Guideline 301 C (MITI I). Method 301 F is new; it is similar to 301 C differing mainly in the inocula employed.

Which of test methods OECD 301B, C, D, F is to be applied depends on the polymer tested. Selecting the respective OECD Test 301 method is well within the general knowledge of the skilled person.

### Enhanced/Modified Ready Biodegradation:

Test duration of OECD TG 301 B, C, D, F is extended to up to 60 days and larger test vessels may be used. Pass criteria: ≥60% mineralisation measured as evolved CO₂ or consumed O₂ in 60 days (10-day window does not apply).

Available test methods: Ready Biodegradability (OECD TG 301 B, C, D, F). 10-day window does not apply here.

Preferred bio-based polymers (and optionally crosslinked polymer networks formed by said bio-based polymers and optional crosslinkers) pass the enhanced/modified ready biodegradation test according to OECD 301B, C, D, F, with ≥60% mineralisation measured as evolved CO₂ or consumed 02 in 60 days. More preferred bio-based polymers (and optionally crosslinked polymer networks formed by said bio-based polymers and optional crosslinkers) pass OECD Test 302C (MITI test II) with ≥70% mineralisation within 14 days; test duration 28 days. Particularly preferred bio-based polymers (and optionally crosslinked polymer networks formed by said bio-based polymers and optional crosslinkers) pass OECD Test 301B, C, D or F with ≥60% mineralisation within 28 days. According to embodiments, bio-based polymer suitable in the present invention (and optionally crosslinked polymer networks formed by said bio-based polymers and optional crosslinkers) pass both OECD Test 302C and OECD Test 301B, C, D or F.

The term “bio-based polymer” as used herein has its ordinary and customary meaning and includes generally synthetic polymers corresponding to polymers produced in a cell of a living organism, synthetic polymers derived from renewable resources, and polymers, biodegradable polymers and biopolymers.

In particular, based on type of polymer and origin of the material of the polymer, the definition of the term “bio-based polymer” encompasses:
A1 Synthetic polymers, wherein the synthetic polymer corresponds to a polymer produced in a cell of a living organism
A2 Synthetic polymers, wherein the monomers are derived or obtained from renewable resources
A3 Synthetic polymers, wherein the synthetic polymer corresponds to a polymer produced in a cell of a living organism, and wherein the monomers are derived or obtained from renewable resources
A4 Biopolymers, i.e. polymers produced in a cell of a living organism
A5 Derivatives of bio-based polymers, as defined below

Based on the degradability of the polymer, the definition of the term “bio-based polymer” encompasses:
B1 Synthetic polymers (irrespective of type of polymer and origin of monomers) exhibiting enhanced/modified ready biodegradability as determined by passing OECD TG 301 B, C, D or F, with test duration extended to 60 days
B2 Synthetic bio-based polymers A1-A3, biopolymers A4 and derivatives A5 of bio-based polymers able to pass OECD Test 302C exhibiting enhanced/modified ready biodegradability as determined by passing OECD TG 301 B, C, D or F, with test duration extended to 60 days
B3 Synthetic polymers (irrespective of type of polymer and origin of monomers) exhibiting inherent biodegradability as determined by passing OECD 302C
B4 Synthetic bio-based polymers A1-A3, biopolymers A4 and derivatives A5 of bio-based polymers exhibiting inherent biodegradability as determined by passing OECD 302C
B5 Synthetic polymers (irrespective of type of polymer and origin of monomers) exhibiting ready biodegradability as determined by passing OECD 301 B, C, D or F
B6 Synthetic bio-based polymers (A1-A3), biopolymers A4 and derivatives A5 of bio-based polymers exhibiting ready biodegradability as determined by passing OECD 301 B, C, D or F

Since the above definitions based on polymer type, monomer origin and degradability overlap, the term “bio-based polymer” also encompasses combinations of the definitions according to (A1-A5) and (B2, B4, B6). According to embodiments, the at least one bio-based polymer is at least derived from renewable resources (A2-A5), and is biodegradable according to B4 or B6. According to a specific embodiment, the bio-based polymer is a biopolymer A4 or derivative A5 thereof, and is biodegradable according to B4. According to another specific embodiment, the bio-based polymer is a biopolymer A4 or derivative A5 thereof, and is biodegradable according to B6.

The term “biopolymer” as used herein is intended to differentiate the polymers suitable in the present invention from petroleum-based polymers, and from synthetic bio-based polymers. As used herein, and consistent with the IUPAC definition, a biopolymer is defined as a macromolecule that is produced in polymeric form in a cell of a living organism. Biopolymers encompass, for example, proteins and peptides such as collagen, gelatin, keratin, silk fibroin, spider silk; α-polysaccharides such as starches; β-polysaccharides such as celluloses, alginates; polyglucosamines such as chitosans, chitins; polynucleotides such as DNA and RNA; polyhydroxyalkanoates such as polyhydroxybutyrate; and other biopolymers such as polyisoprene (rubber), suberin, melanin and lignin.

“Derivatives of bio-based polymers” as used herein refers to bio-based polymers which have been modified as compared to the bio-based polymer as synthesized or biopolymer as produced by and obtained from, respectively, a cell of a living organism. Derivates of bio-based polymers encompasses modification on basis of monomer structure, and modification of polymer structure.

Derivates of polymers comprising modification of the monomer structure include:
Masking of functional groups, in order to render inert or decrease the reactivity of a particular functional group on at least part of the monomers of the polymer. Examples of the masking of functional groups include methylation or ethylation of hydroxy-functional groups of the repeating units of polysaccharides. Another example is the acetylation of amine-functional groups of chitosan repeating units as disclosed herein. For functional groups present on each repeating unit, masking is feasible in an amount up to about 80% of the repeating units. For example, up to 80% of the repeating units of chitosan, or up to 80% of the amine-functional groups of chitosan may be modified by masking. Typically, 0.05-60%, more typically 0.05-40% or 0.1-30%, in particular 0.2-20% such as 0.3-15% or 0.5-10% of the repeating units of a bio-based polymer such as for example chitosan may be modified by masking of functional groups. For functional groups not present on each repeating unit, for example as is the case for proteins, masking will be feasible to a lesser degree. For example, up to about 10% of the repeating units of gelatin may be modified by masking. Termed differently, 0.05-60%, more typically 0.05-40% or 0.1-30%, in particular 0.2-20% such as 0.3-15% or 0.5-10% of the functional groups (sum of hydroxy-functional, amine-functional, thio-functional, carboxy-functional groups) of a protein such as for example gelatin may be modified by masking of functional groups.
Demasking, in order to increase the reactivity of a particular group on at least part of the monomers of the polymer, or to convert such group to a functional group. Examples of demasking include the deacetylation of chitin repeating units to give chitosan repeating units, or amine-functional groups, respectively. Demasking amounts or degrees are considered herein essentially reciprocal to the masking degrees mentioned above. Accordingly, at least 20% of the repeating units of chitin, or up to 20% of the aminoacetyl-functional groups of chitin may be modified by demasking. Typically, 40-99.95%, more typically 60-99.95% or 70-99.9%, in particular 80-99.8% such as 85-99.7% or 90-99.5% of the repeating units of a bio-based polymer such as for example chitosan may be modified by demasking of functional groups.
Activating functional groups, in order to provide the polymer(s) with specific reactivities. Examples of the activation of functional groups include reacting functional groups of the polymer(s) to give pendant or terminal self-reactive groups, pendant or terminal unsaturated groups, and pendant or terminal groups susceptible to Michael reactions such as aza-Michael reactions, thio-Michael reactions or oxa-Michael reactions. Specific examples of activation include reacting amine-functional groups or thio-functional groups or hydroxy-functional groups of the polymer(s) with (meth)acrylic anhydride or glycidyl (meth)acrylate to give olefinolyl groups, i.e. (meth)acryloyl groups. Modification amounts or degrees by activation of functional groups are analogous to the masking degrees mentioned above. For example, up to 80% of the repeating units of a polysaccharide such as chitosan, or up to 10% of the structural units of a protein such as gelatin may be modified by activating functional groups. Termed differently, typically 0.05-60%, more typically 0.05-40% or 0.1-30%, in particular 0.2-20% such as 0.3-15% or 0.5-10% of the functional groups of a bio-based polymer such as for example chitosan (amine-functional groups) or gelatin (sum of hydroxy-functional, amine-functional, thio-functional, carboxy-functional groups) may be modified by activation of functional groups.
Modification of functional groups in order to change the charge density of the polymer(s). An example of changing the charge density is reacting carboxy-functional groups of the polymer(s) with for example ethylene diamine to give groups which at moderate to low pH will be positively charged, or reacting hydroxyl-functional groups for example with betaine, to give groups which irrespective of the pH will be positively charged. Positively charged polymers, also referred to herein as “cationized polymers” may provide for enhanced electrostatic interactions with keratin fibers such as human hair. Another example of changing the charge density is reacting carboxy-functional groups of the polymer(s) with for example 2-aminoethane sulfonic acid, or reacting amino-functional groups of the polymer(s) with a dicarboxylic acid, to give groups carboxy-functional which at moderate to high pH will be negatively charged. Negatively charged polymers, also referred to herein as “anionized polymers” may provide for enhanced electrostatic interactions with cationic layers on keratin fibers such as human hair.
Modification of functional groups in order to change the water solubility of polymer(s). An example of changing the water solubility is the sulfonation of lignin. It is noted that masking, demasking and activating as well will have an impact on the water solubility, but the effect of masking, demasking and activating is primarily a change of the reactivity of the polymer(s) to itself, a different polymer or a crosslinker, and not a change of a physical parameter such as water solubility.
Modification of functional groups in order to change the rheological properties, i.e. the viscosity of polymer(s). It is noted that masking, demasking and activating as well may have an impact on the rheological properties, so that there may be an overlap in this context. An example of changing the rheological properties of a polymer by modification of functional groups is the acetylation (masking) of amino-functional groups of gelatin.

Derivates of polymers comprising modification of the polymer structure include:
Changing the molecular weight of individual polymer chains. Such modifications include for example reducing the weight of protein chains by enzymatic treatment or hydrolysis. An example of such modification is hydrolysis of collagen to gelatin.
Changing the crosslink density of individual polymer chains. Such modifications include for example treatment of polysaccharides with amylases.
Changing the secondary structure of the polymers. Such modifications include for example denaturation.

Considering that any modification on basis of monomer structure may have occurred prior to the synthesis of a synthetic polymer, and any modification of the polymer structure typically is not applicable to synthetic polymers, modification of the monomer structure by masking or demasking, and modification of the polymer structure are applicable essentially to the biopolymers. Modifications like activation of functional groups and changing physical parameters such as charge density or water solubility are applicable to the bio-based polymers in general.

The term “biogenic” as used herein refers to a substance produced by a living organism or resulting from the activity of a living organism. The term encompasses constituents, secretions and metabolites of living organisms of contemporary age, but excludes constituents, secretions and metabolites of geologic age.

The term “organism” as used herein encompasses any live forms that are able to reproduce and grow. In particular, the term “organism” encompasses animals, plants, fungi, eukaryotic and prokaryotic microorganisms, and superorganisms.

The term “water-soluble” as used herein refers to a solubility at 25° C. in the aqueous medium of the composition for treating keratin fibers of at least 1% by weight, in particular at least 5% by weight, for example at least 10% by weight such as at least 25% by weight. A polymer being “water-soluble” represents a distinction from pigments, which per definition are non-dissolved solids.

The term “crosslinker” as used herein refers to compounds able to initiate crosslinking of the at least one bio-based polymer. In particular, crosslinkers may catalyze or initiate the crosslinking by promoting the formation of covalent bonds, hydrogen bonding, electrostatic or ionic interaction, ionic gelation, etc.

Examples of crosslinkers able to catalyze crosslinking are compounds such as photoinitiators and thermal crosslinkers, and other compounds able to initialize free-radical polymerization.

Crosslinkers suitable for promoting ionic gelation provide electrical charges complementary to and interacting with electrical charges provided by functional groups of particular polymer(s), which electrostatic interaction promotes immobilizing of an entangled matrix comprising the particular polymer(s) and crosslinkers on the keratin fibers. Examples of crosslinkers promoting ionic gelation include for instance polyvalent ions.

Crosslinkers comprising functional groups complementary to functional groups of the polymer(s) generally are compounds able to react with functional groups present on the at least one bio-based polymer, present on any polymer other than the bio-based polymer(s), if present, and/or present on the keratin fibers. A particular crosslinker may comprise one type of functional groups, or more than one type of functional groups, such as hydroxy, amine, carboxy, mercapto, isocyanate, or olefinoyl. Furthermore, the number of functional groups present on each individual crosslinker molecule is at least two, preferably at least three, at least four, at least five, or at least six functional groups, which groups are able to react with functional groups of the polymer(s) (optionally with functional groups present on the keratin fibers). In view of the crosslinking reaction, the crosslinkers comprising complementary functional groups will become integrated into the crosslinked polymer structure.

According to further embodiments, the crosslinkers may induce the crosslinking of functional groups without becoming integrated into the crosslinked polymeric structure. Examples of such crosslinkers include for example carbodiimides. Carbodiimides are able to react with carboxy-functional groups via formation of an unstable o-acylisourea intermediate, which intermediate subsequently may react with a primary amine group, thereby forming an amide bond. In this latter reaction step, the remnant of the carbodiimide moiety represents a leaving group, which does not become integrated into the crosslinked polymeric structure.

Still further examples of crosslinkers encompass silanes as disclosed herein

Crosslinkers can be distinguished from the polymers and in particular biopolymers in terms of their molecular weight, or the polymer backbone or structural units, respectively, or the density of particular functional groups. For example, crosslinkers able to initialize free-radical polymerization, carbodiimides, and many of the crosslinkers providing for ionic gelation are small molecules. Crosslinkers comprising complementary functional groups such as polythiols and polyisocyanates as well exhibit molecular weights significantly lower the average molecular weights of the polymers. Crosslinkers such as polyethylene imines, which may be oligomers or small polymers, can be distinguished from bio-based polymers and biopolymers in terms of the structural units and the number of amine-functional groups relative to the weight of an individual polymer chain. Silane-functional crosslinkers obviously can be distinguished from the polymers and in particular biopolymers in terms of the silane-functional groups.

The term “polymer” as used herein denotes homopolymers, copolymers or terpolymers. Further, the term “polymer” as used herein denotes macromolecules comprised of or essentially comprised of structural units, and comprising more than 20, usually more than 50, more typically 80 or more, in particular 100 or more of said structural units. These structural units or repeating units account for at least 80% by weight of an individual polymeric chain, which polymeric chain accordingly may comprise constituents other than the repeating units, which constituents account for up to 20% by weight of an individual polymeric chain. Polymers may comprise up to several thousand or up to several million of structural units. Copolymers comprise two different types of structural units, and terpolymers comprise three different types of structural units. The term “oligomer” as used herein denotes macromolecules made of structural units, and comprising at least 5 and up to 50, usually 40 or less, more typically 30 or less of said structural units.

Termed differently, the term “polymer” as used herein denotes macromolecules comprised of or essentially comprised of structural units, and having a weight average polymer molecular weight M_{w} in the range of 10,000 g/mol to 5,000,000 g/mol, typically in the range 15,000 g/mol to 1,000,000 g/mol, for example in the range of 20,000 g/mol to 500,000 g/mol, such as 30,000 g/mol to 300,000 g/mol. Analogously, “oligomers as used herein have a weight average oligomer molecular weight M_{w} in the range of 500,000 g/mol to 8,000 g/mol, typically in the range 1,000 g/mol to 7,500 g/mol, for example in the range of 1,500 g/mol to 5,000 g/mol, such as 2,000 g/mol to 4,000 g/mol.

The terms “repeating unit” and “monomer” are used herein interchangeably, and denote the structural units of which the polymers are composed. If the emphasis is on the structural unit required for synthesizing a particular polymer, the term “monomer” is preferred herein. If the emphasis is on the remnant of the structural unit present in a particular macromolecule in polymeric form, the term “repeating unit” is preferred. As is well-known in the art, the monomers used for synthesizing a particular biopolymer are identical, but the repeating units in the resulting polymers may differ due to branching. For example, the biopolymer “starch” is formed by polymerization of glucose monomers via α-1,4-glycosidic bonds, with a part of the glycosidic repeating units branched via α-1,6-glycosidic bonds.

Proteins may be homopolymers or heteropolymers. Homopolymers are comprised of identical amino acids, while protein heteropolymers are comprised of different amino acids, and in the case of biopolymers have a specific amino acid sequence, i.e. primary structure. As used herein in the context of protein heteropolymers, the terms “monomer” and “repeating unit” denote an amino acid. Accordingly, and deviating from the usual meaning of these terms, the “monomers” or “repeating units” of a protein heteropolymer are not necessarily identical, but have formulas corresponding to one of the natural occurring amino acids.

The term “polymer type” as used herein denotes polymer classes based on the structural units and their linkage. Polyesters, polyamides, proteins and polysaccharides, for example, are different polymer types. Another example of bio-based polymers of different types is carboxy-functional polysaccharides and amino-functional polysaccharides. Vice versa, proteins having different amino acid sequences, such as gelatin and albumin, for example, are different bio-based polymers of the same polymer type. In particular when used in the context of a polymer produced in a cell of a living organism, the term “polymer type” encompasses derivatives of such polymers as defined herein.

The term “essentially free” of a particular component denotes an amount of less than 1% by weight of the respective component in an entity such as a composition, based on the total weight of the entity. In particular, the term “essentially free” denotes an amount of less than 0.5% by weight, for example less than 0.1% by weight, such as 0.01% by weight of the respective component in the entity.

### DETAILED DESCRIPTION

Embodiments of the instant invention generally relate to coatings of at least one bio-based polymer, optionally with colored pigment microparticles, on the surfaces of keratin fibers, and especially on mammalian keratin fibers. The coating is formed by treatment of the keratin fibers, especially keratin fibers such as hair with the embodiments of the above described composition. The coating is wash resistant yet can be removed without damage to the keratin fibers such as hair. The embodiments of the composition minimize or avoid damage to keratin proteins within the keratin fibers, particularly after repeated dying events. The embodiments of the composition limit irritation of the scalp which may result from application of known hair dye compositions. The present invention is directed to embodiments of compositions for coloration of keratin fibers in such a way that the color can be applied and will remain until it is desired to remove the color. This makes the treatment process more pleasurable for the user and or stylist. It is also desired that the results are predictable, enabling the users to achieve their target hair color result.

The composition, method and coating aspects of the invention are directed to embodiments of a composition that are adapted to provide coatings on the surfaces of keratin fibers, especially hair strands. According to embodiments, the composition is adapted to provide colored coatings on the surfaces of keratin fibers. The colored coating embodiments have remanence that enables them to remain in somewhat to substantial to essential original composition especially upon the hair embodiment of keratin fibers through at least a series of washings with diluted aqueous media containing soap and/or shampoo. The compositions minimally alter the keratin fibers upon their application. As used herein, the term “minimally alters the keratin fibers” generally means that after removal of the composition the keratin fibers are returned to a substantially unaltered state.

The embodiments of the composition form a coating that surprisingly is durable and resistant to repeated washings with ordinary shampoos, soap, detergent and water. The bio-based polymers and optional crosslinkers in situ form covalent, coordinate, entanglement, electrostatic, ionic and/or dipolar linkages. It is believed that the forming of in situ linkages produces an arrangement of coating, optional microparticles and keratin fibers that are interconnected and develop the unexpected, surprising long standing remanence.

Embodiments of the invention also include methods for preparation of the composition, kits for storage and delivery of the composition, methods for application of the composition to keratin fibers such as hair, as well as coatings on keratin fibers.

### I. The Composition for Treating a Keratin Fiber

The composition comprises components for production of a remanent, in particular a remanent colored coating on keratin fibers such as hair. The components interact in situ to provide covalent bonding or electrostatic interactions among the components and the keratin fibers.

### A the at Least One Bio-Based Polymer

The compositions described herein comprise as an essential component at least one bio-based polymer. Bio-based polymers as used herein generally encompass biopolymers, i.e. polymers produced in a cell of a living organism, as well as synthetic polymers produced from materials derived or obtained from renewable resources and/or being biodegradable. According to embodiments, the composition comprises two types of bio-based polymers, or even more than two types of bio-based polymers.

### A1 Synthetic Bio-Based Polymers and Biopolymers

The synthetic bio-based polymers encompass synthetic polymers derived from renewable resources, synthetic polymers corresponding in structure to biopolymers, and polymers having a minimum biodegradability. The bio-based polymers have a weight average polymer molecular weight M_{w} in the range of 10,000 g/mol to 5,000,000 g/mol, typically in the range 15,000 g/mol to 1,000,000 g/mol, for example in the range of 20,000 g/mol to 500,000 g/mol, such as 30,000 g/mol to 300,000 g/mol. In addition, the bio-based polymers exhibit a solubility in the aqueous medium of the composition for treating keratin fibers of at least 1% by weight. In particular, the bio-based polymers exhibit a solubility in the aqueous medium of the composition at least 5% by weight, for example at least 10% by weight such as at least 25% by weight.

Based on type of polymer and origin of the material of the polymer, the definition of the term “bio-based polymer” encompasses:

A1 Synthetic polymers, wherein the synthetic polymer corresponds to a polymer produced in a cell of a living organism. Synthetic polymers A1 accordingly in structure correspond to biopolymers, but are not obtained in polymeric form from renewable resources, and the materials used for synthesizing the polymers are not necessarily derived from renewable resources. Examples of synthetic bio-based polymers A1 include macromolecules as indicated for the biopolymers, for example, proteins and peptides such as collagen, gelatin, keratin, silk fibroin, spider silk; α-polysaccharides such as starches; β-polysaccharides such as celluloses, alginates; polyglucosamines such as chitosans, chitins; polynucleotides such as DNA and RNA; polyhydroxyalkanoates such as polyhydroxybutyrate; gums such as polyisoprenes (rubber); and other polymers such as suberin, melanin or lignin.
A2 Synthetic polymers, wherein the monomers are derived or obtained from renewable resources. Accordingly, the materials for synthesizing polymers A2 are derived or obtained from renewable resources, but the polymers per se not necessarily are produced in a cell of a living organism. Examples of synthetic bio-based polymers A2 include for example polyethers, polyesters, polyether-esters, polyamides, polyesteramides, polyolefines, polyurethanes, polyacrylates, or poly(lactic acid) (PLA), the monomers of which are derived or obtained from renewable resources.
A3 Synthetic polymers, wherein the synthetic polymer corresponds to a polymer produced in a cell of a living organism, and wherein the monomers are derived or obtained from renewable resources. Synthetic polymers A3 accordingly in structure correspond to biopolymers, the materials used for synthesizing the polymers are derived or obtained from renewable resources, but the polymers per se are obtained synthetically. Synthetic polymers A3 may differ from biopolymers, for example, with respect to average molecular weight or molecular weight distribution, or polydispersity index. Examples of synthetic bio-based polymers A3 include the macromolecules as indicated for the biopolymers and synthetic polymers A1.
A4 Biopolymers. Biopolymers per definition are macromolecules produced in polymeric form in a cell of a living organism. Biopolymers encompass, for example, proteins and peptides such as collagen, gelatin, keratin, silk fibroin, spider silk; α-polysaccharides such as starches; β-polysaccharides such as celluloses, alginates; polyglucosamines such as chitosans, chitins; polynucleotides such as DNA and RNA; polyhydroxyalkanoates such as polyhydroxybutyrate; and other biopolymers such as polyisoprene (rubber), suberin, melanin and lignin. Preferred gelatins exhibit a weight average polymer molecular weight M_{w} in the range of 20,000 g/mol to 100,000 g/mol, for example in the range of 50,000 g/mol to 80,000 g/mol. Preferred chitosans exhibit a weight average polymer molecular weight M_{w} in the range of 50,000 g/mol to 500,000 g/mol, for example in the range of 100,000 g/mol to 400,000 g/mol, typically in the range of 150,000 g/mol to 300,000 g/mol. Preferred alginates exhibit a weight average polymer molecular weight M_{w} in the range of 60,000 g/mol to 250,000 g/mol, for example in the range of 90,000 g/mol to 180,000 g/mol.
A5 Derivatives of bio-based polymers, as defined herein below. Generally, derivatives of the bio-based polymers differ from the bio-based polymers as synthesized or biopolymers as obtained from a cell of a living organism by modification on basis of monomer structure, and/or modification of polymer structure.

According to embodiments, the at least one bio-based polymer is a polymer A2 or A3 made of material derived or obtained from renewable resources, or is a biopolymer A4, or a derivative A5 of a polymer A2, A3 or A4.

Based on the degradability of the polymer, the definition of the term “bio-based polymer” encompasses:

B1 Synthetic polymers (irrespective of type of polymer and origin of monomers) exhibiting enhanced/modified ready biodegradability as determined by passing OECD TG 301 B, C, D or F, with test duration extended to 60 days
B2 Synthetic bio-based polymers A1-A3, biopolymers A4 and derivatives A5 of bio-based polymers able to pass OECD Test 302C exhibiting enhanced/modified ready biodegradability as determined by passing OECD TG 301 B, C, D or F, with test duration extended to 60 days
B3 Synthetic polymers (irrespective of type of polymer and origin of monomers) exhibiting inherent biodegradability as determined by passing OECD 302C
B4 Synthetic bio-based polymers A1-A3, biopolymers A4 and derivatives A5 of bio-based polymers exhibiting inherent biodegradability as determined by passing OECD 302C
B5 Synthetic polymers (irrespective of type of polymer and origin of monomers) exhibiting ready biodegradability as determined by passing OECD 301 B, C, D or F
B6 Synthetic bio-based polymers A1-A3, biopolymers A4 and derivatives A5 of bio-based polymers exhibiting ready biodegradability as determined by passing OECD 301 B, C, D or F

According to embodiments, the at least one bio-based polymer is a polymer A2 or A3 made of material derived or obtained from renewable resources, or is a biopolymer A4, or a derivative A5 of a polymer A2, A3 or A4, which polymer or derivative thereof further is biodegradable according to B2, B4 or B6. In particular, a bio-based polymer (A1-A3), biopolymer A4 or derivative A5 thereof may be able to pass OECD Test 301B, C, D or F, or able to pass OECD Test 302C, or able to pass both OECD Test 301B, C, D or F and OECD Test 302C.

According to embodiments, the crosslinked matrix of synthetic polymers (irrespective of type of polymer and origin of monomers) may exhibit enhanced/modified ready biodegradability as determined by passing OECD TG 301 B, C, D or F, with test duration extended to 60 days. According to embodiments, the crosslinked matrix of synthetic polymers (irrespective of type of polymer and origin of monomers) may exhibit inherent biodegradability as determined by passing OECD 302C. According to embodiments, the crosslinked matrix of synthetic polymers (irrespective of type of polymer and origin of monomers) may exhibit ready biodegradability as determined by passing OECD 301 B, C, D or F. According to embodiments, the crosslinked matrix of synthetic polymers (irrespective of type of polymer and origin of monomers) may be able to pass both OECD Test 301B, C, D or F and OECD Test 302C.

According to embodiments, the crosslinked matrix of synthetic bio-based polymers A1-A3, biopolymers A4 and/or derivatives A5 may exhibit enhanced/modified ready biodegradability as determined by passing OECD TG 301 B, C, D or F, with test duration extended to 60 days. According to embodiments, the crosslinked matrix of synthetic bio-based polymers A1-A3, biopolymers A4 and/or derivatives A5 may exhibit inherent biodegradability as determined by passing OECD 302C. According to embodiments, the crosslinked matrix of synthetic bio-based polymers A1-A3, biopolymers A4 and/or derivatives A5 may exhibit ready biodegradability as determined by passing OECD 301 B, C, D or F. According to embodiments, the crosslinked matrix of synthetic bio-based polymers A1-A3, biopolymers A4 and/or derivatives A5 may be able to pass both OECD Test 301B, C, D or F and OECD Test 302C.

### A2 Derivatives of Bio-Based Polymers

“Derivatives of bio-based polymers” as used herein refers to bio-based polymers which have been modified as compared to the bio-based polymer as synthesized or biopolymer as produced by and obtained from, respectively, a cell of a living organism. Derivates of bio-based polymers encompasses modification on basis of monomer structure, and modification of polymer structure.

Derivates of polymers comprising modification of the monomer structure include:
Masking of functional groups, in order to render inert or decrease the reactivity of a particular functional group on at least part of the monomers of the polymer. Examples of the masking of functional groups include methylation or ethylation of hydroxy-functional groups of the repeating units of polysaccharides. Another example is the acetylation of amine-functional groups of chitosan repeating units as disclosed herein. For functional groups present on each repeating unit, masking is feasible in an amount up to about 80% of the repeating units. For example, up to 80% of the repeating units of chitosan, or up to 80% of the amine-functional groups of chitosan may be modified by masking. Typically, 0.05-60%, more typically 0.05-40% or 0.1-30%, in particular 0.2-20% such as 0.3-15% or 0.5-10% of the repeating units of a bio-based polymer such as for example chitosan may be modified by masking of functional groups. For functional groups not present on each repeating unit, for example as is the case for proteins, masking will be feasible to a lesser degree. For example, up to about 10% of the repeating units of gelatin may be modified by masking. Termed differently, 0.05-60%, more typically 0.05-40% or 0.1-30%, in particular 0.2-20% such as 0.3-15% or 0.5-10% of the functional groups (sum of hydroxy-functional, amine-functional, thio-functional, carboxy-functional groups) of a protein such as for example gelatin may be modified by masking of functional groups.
Demasking, in order to increase the reactivity of a particular group on at least part of the monomers of the polymer, or to convert such group to a functional group. Examples of demasking include the deacetylation of chitin repeating units to give chitosan repeating units, or amine-functional groups, respectively. Demasking amounts or degrees are considered herein essentially reciprocal to the masking degrees mentioned above. Accordingly, at least 20% of the repeating units of chitin, or up to 20% of the aminoacetyl-functional groups of chitin may be modified by demasking. Typically, 40-99.95%, more typically 60-99.95% or 70-99.9%, in particular 80-99.8% such as 85-99.7% or 90-99.5% of the repeating units of a bio-based polymer such as for example chitosan may be modified by demasking of functional groups.
Activating functional groups, in order to provide the polymer(s) with specific reactivities. Examples of the activation of functional groups include reacting functional groups of the polymer(s) to give pendant or terminal self-reactive groups, pendant or terminal unsaturated groups, and pendant or terminal groups susceptible to Michael reactions such as aza-Michael reactions, thio-Michael reactions or oxa-Michael reactions. Specific examples of activation include reacting amine-functional groups or thio-functional groups or hydroxy-functional groups of the polymer(s) with (meth)acrylic anhydride or glycidyl (meth)acrylate to give olefinolyl groups, i.e. (meth)acryloyl groups. Modification amounts or degrees by activation of functional groups are analogous to the masking degrees mentioned above. For example, up to 80% of the repeating units of a polysaccharide such as chitosan, or up to 10% of the structural units of a protein such as gelatin may be modified by activating functional groups. Termed differently, typically 0.05-60%, more typically 0.05-40% or 0.1-30%, in particular 0.2-20% such as 0.3-15% or 0.5-10% of the functional groups of a bio-based polymer such as for example chitosan (amine-functional groups) or gelatin (sum of hydroxy-functional, amine-functional, thio-functional, carboxy-functional groups) may be modified by activation of functional groups.
Modification of functional groups in order to change the charge density of the polymer(s). An example of changing the charge density is reacting carboxy-functional groups of the polymer(s) with for example ethylene diamine to give groups which at moderate to low pH will be positively charged, or reacting hydroxyl-functional groups for example with betaine, to give groups which irrespective of the pH will be positively charged. Positively charged polymers, also referred to herein as “cationized polymers” may provide for enhanced electrostatic interactions with keratin fibers such as human hair. Another example of changing the charge density is reacting carboxy-functional groups of the polymer(s) with for example 2-aminoethane sulfonic acid, or reacting amino-functional groups of the polymer(s) with a dicarboxylic acid, to give groups carboxy-functional which at moderate to high pH will be negatively charged. Negatively charged polymers, also referred to herein as “anionized polymers” may provide for enhanced electrostatic interactions with cationic layers on keratin fibers such as human hair.
Modification of functional groups in order to change the water solubility of polymer(s). An example of changing the water solubility is the sulfonation of lignin. It is noted that masking, demasking and activating as well will have an impact on the water solubility, but the effect of masking, demasking and activating is primarily a change of the reactivity of the polymer(s) to itself, a different polymer or a crosslinker, and not a change of a physical parameter such as water solubility.
Modification of functional groups in order to change the rheological properties, i.e. the viscosity of polymer(s). It is noted that masking, demasking and activating as well may have an impact on the rheological properties, so that there may be an overlap in this context. An example of changing the rheological properties of a polymer by modification of functional groups is the acetylation (masking) of amino-functional groups of chitosans.

Derivates of polymers comprising modification of the polymer structure include:
Reducing the molecular weight of individual polymer chains. Such modifications include for example reducing the weight of protein chains by enzymatic treatment or hydrolysis. An example of such modification is hydrolysis of collagen to gelatin.
Changing the crosslink density of individual polymer chains. Such modifications include for example treatment of polysaccharides with amylases.
Changing the secondary structure of the polymers. Such modifications include for example denaturation.

Modification of the polymer structure will affect the molecular weight parameters of a given polymer sample. Molecular weight parameters of polymers are determined using size exclusion chromatography in accordance with international accepted standard methods.

According to embodiments, the derivatives of the polymers comprise more than one type of monomer modification. In embodiments, the polymer derivatives may comprise 0.1-30% activated repeating units, and 0.1-30% masked repeating units or cationized or anionized repeating units. In other embodiments, the polymer derivatives may comprise 70-99.9% demasked repeating units and 0.1-30% activated repeating units. In particular examples, the polymers may comprise may comprise a total of 0.5-60% masked, activated and/or cationized or anionized repeating units, for example a total of 1-30% such as 2-20% masked, activated and/or cationized or anionized repeating units. A specific example of a biopolymer derivative is gelatin (i.e. hydrolyzed collagen) comprising 0.5-15% masked repeating units, 0.5-15% activated repeating units and optionally 0.5-15% cationized repeating units. A particular example is a derivatized gelatin comprising 5-10% masked repeating units, 2-12% activated repeating units and optionally 5-12% cationized repeating units. Another specific example of a biopolymer derivative is chitosan (i.e. demasked chitin) comprising 0.5-15% activated repeating units and optionally 0.5-15% cationized repeating units. A particular example is a derivatized chitosan comprising 0.2-12% activated repeating units and optionally 5-12% cationized repeating units.

A3 Examples of bio-based Polymers

In addition to biopolymers and bio-based polymers exemplified above, further examples of biopolymers and bio-based polymers, respectively, include Polysaccharides, Polyethylenglycol, polyanhydrides, hyaluronic acid, natural polyaminoacids, phosphorous based polymers, alginic acid, xantham gum, gum Arabic, starch, cellulose, collagen, pectin, carrageenan, polyesters, polylactides, polyglycolides, poly(lactide-co-glycolite), polycaprolactone, polyhdyroxyalkanoates, polyester amides, polyorthoesters, polyanhydrides, polyalkylcyanoacrylates, polyorthoesters, mussel adhesive protein, Poly(γ-glutamic acid), Poly lysine, Cellulose acetate, Cellulose acetate butyrate, Cellulose acetate propionate, Carboxymethyl cellulose, Cellulose nitrate, Hydroxyethyl cellulose, Caddisfly adhesive protein, Sandcastle worm protein, Barnacle protein, Fibrin, Gelatin, Gelatin-resocinol-formaldehyde, Poly(hydroxydodecanoate), Poly(hydroxydecanoate), Poly(hydroxynonanoate), Poly(hydroxyoctanoate), Poly(hydroxypropionate), Poly(hydroxyvalerate), Poly(hydroxyundecanoate), Poly(hydroxybutyrate), Polyamide, Poly(alkylene alkanoate), Poly(alkylene dicarboxylate), Poly(aspartic acid), Poly(butylene adipate), Poly(butylene carbonate), Poly(butylene sebacate), Poly(cyclohexene carbonate), Poly caprolactone, Poly(lactide), Poly(d-lactic acid), Polyethylene, Poly(ethylene carbonate), Poly(ethylene oxalate), Poly(ethylene succinate), Poly(ethylene terephthalate), Poly(ester urethane urea), Polyglycolide, poly(glycolic acid), Polyglycerol sebacate, Polyhydroxyalkanoate, Polylactide, poly(lactic acid), Poly(β-malic acid), Poly(ortho ester), Polyphthalamide, Poly(propylene fumarate), Poly(β-propiolactone), Poly(propylene succinate), Poly(propylene terephthalate), Poly succinimide, Poly(tetramethylene adipate-coterephthalate), Poly(tetramethylene carbonate), Poly(trimethylene carbonate), Poly(tetramethylene succinate), Poly(butylene succinate), Poly(trimethylene terephthalate), Polyurethane, Poly(vinyl alcohol).

A4 Polymers Other than Bio-Based Polymers

The compositions according to the present invention comprise at least one bio-based polymer, and may comprise different types of bio-based polymers. The amount of the bio-based polymers is at least 80% by weight, based on the sum of the dry weight of bio-based polymers and polymers other than bio-based polymers. According to embodiments, the amount of the bio-based polymers is at least 90% by weight, or at least 95% by weight, for example at least 98% by weight, based on the sum of the dry weight of bio-based polymers and polymers other than bio-based polymers. The term “polymer other than bio-based polymer” as used herein denotes any macromolecule irrespective of its function, which macromolecule is comprised of, or essentially comprised of repeating units, and has a weight average polymer molecular weight M_{w} in the range of 10,000 g/mol to 5,000,000 g/mol, typically in the range 15,000 g/mol to 1,000,000 g/mol, for example in the range of 20,000 g/mol to 500,000 g/mol, such as 30,000 g/mol to 300,000 g/mol.

Polymers other than bio-based polymers include but are not limited to polymers of appropriate monomeric units such as but not limited to one or more olefin monomers, ester units of diacids/diol monomers or of hydroxy acid monomers, ether monomeric units, thioether monomeric units, polyol monomeric units, alkylene oxide monomeric units, alkylene imine monomeric units, urethane monomeric units urea monomeric units, amide units of diacid/diamine monomers; hydroxylated polyester, acrylate functionalized polyester, polyester polyurethane acrylic copolymer, polyurethane-polyglycol copolymer, polycarbonate diols, styrene-allyl alcohol copolymer, ketone resins; as well as other repeating residues based on carbon or carbon in combination with other atoms such as oxygen and/or nitrogen, and any combination thereof. Additional polymers include but are not limited to non-polar olefinic polymers, polar, non-protonic olefinic polymers, vinyl polymers, polyethers, polycondensates, block polymers and any compound with repeating carbon unit residues. Preferably the precursor organic polymers are polyolefins including polyvinyl compounds, polyesters, polyethers, polyurethanes or polyamides or any combination thereof. More preferably, the organic polymers are polyolefins including polyvinyl compounds, polyesters or polyurethanes or any combination thereof. Especially more preferably, the organic polymers are polyolefins, polyvinyl compounds or polyesters.

Organic polymers containing acid groups may be developed from any monomeric unit containing acid groups such as carboxylic acid, sulfonic acid, sufinic acid, phosphoric acid. The acidic units may be combined with non-acidic units which are hydrophilic or hydrophobic to provide appropriate precursor organic polymers. Such polymers are described in the following passages.

Organic polymers may include copolymers of (meth)acrylic acid and of at least one linear, branched or cyclic (cycloaliphatic or aromatic) (meth)acrylic acid ester monomer and/or of at least one linear, branched or cyclic (cycloaliphatic or aromatic) mono- or disubstituted (meth)acrylic acid amide monomer.

Included are copolymers such as acrylic acid/ethyl acrylate/N-tert-butylacrylamide terpolymers such as the product sold under the name Ultrahold 8 and that sold under the name Ultrahold Strong by the company BASF; (meth)acrylic acid/tert-butyl (meth)acrylate and/or isobutyl (meth)acrylate/C₁-C₄ alkyl (meth)acrylate copolymers such as the acrylic acid/tert-butyl acrylate/ethyl acrylate terpolymer sold by the company BASF under the name Luvimer 100P; (meth)acrylic acid/ethyl acrylate/methyl methacrylate terpolymers and tetrapolymers such as the ethyl acrylate/methyl methacrylate/acrylic acid/methacrylic acid copolymer such as the product sold under the name Amerhold DR-25 by the company Amerchol; methyl methacrylate/butyl or ethyl acrylate/hydroxyethyl or 2-hydroxypropyl acrylate or methacrylate/(meth)acrylic acid tetrapolymers such as the methyl methacrylate/butyl acrylate/hydroxyethyl methacrylate/methacrylic acid tetrapolymers sold by the company Rohm & Haas under the name Acudyne 255.

Additional examples of organic polymers include copolymers of acrylic acid and of C1-C4 alkyl methacrylate and terpolymers of vinylpyrrolidone, of acrylic acid and of C1-C20 alkyl, for example lauryl, methacrylate, such as that sold by the company ISP under the name Acrylidone M and the copolymer of methacrylic acid and of ethyl acrylate sold under the name Luvimer MAEX by the company BASF.

Yet other examples of organic polymers include amphoteric copolymers such as N-octylacrylamide/methyl methacrylate/hydroxypropyl methacrylate/acrylic acid/tert-butylaminoethyl methacrylate copolymers, in particular that sold under the name Amphomer by the company National Starch, or the copolymer Lovocryl L47 sold by the same company.

Additional examples of organic polymer include copolymers of (meth)acrylic acid and of (meth)acrylic acid esters or amides furthermore containing linear, branched or cyclic (cycloaliphatic or aromatic, which may or may not be substituted) vinyl esters, such as vinyl acetate; vinyl propionate; vinyl esters of branched acid such as vinyl versatate; vinyl esters of substituted or unsubstituted benzoic acid; these copolymers may furthermore also contain groups resulting from the copolymerization with styrene, alpha-methylstyrene or a substituted styrene. Other examples include copolymers of (meth)acrylic acid and of at least one olefinic monomer chosen from vinyl esters such as those mentioned above and containing no (meth)acrylic acid acrylamide or ester monomer. These copolymers may also contain olefinic groups resulting from the copolymerization with styrene, ·alpha·-methylstyrene, a substituted styrene and optionally monoethylenic monomers such as ethylene.

Still other examples include copolymers of vinyl monoacid such as crotonic acid and vinylbenzoic acid and/or of allylic monoacid such as allyloxyacetic acid.

Organic polymers include copolymers of crotonic acid containing vinyl acetate or propionate units in their chain and optionally of other monomers such as allylic or methallylic esters, vinyl ethers or vinyl esters of a saturated, linear or branched carboxylic acid containing a long hydrocarbon chain, such as those containing at least 5 carbon atoms, it being possible for these polymers optionally to be grafted and crosslinked, or alternatively a vinyl, allylic or methallylic ester of an alpha- or beta-cyclic carboxylic acid. These copolymers may also contain olefinic groups resulting from the copolymerization with styrene, alpha-methylstyrene, a substituted styrene and optionally monoethylenic monomers such as ethylene.

Organic polymers include vinyl polymers such as vinyl acetate/crotonic acid/polyethylene glycol copolymers such as that sold by the company Hoechst under the name “Aristoflex A”; vinyl acetate/crotonic acid copolymers such as that sold by the company BASF Additional examples of precursor organic polymers include the polyolefins, polyvinyls, polyesters, polyurethanes, polyethers, polycondensates and natural polymers of the following passages.

Additional organic polymers include but are not limited to homopolymers and copolymers of olefins; cycloolefins; butadiene; isoprene; styrene; vinyl ethers, esters, or amides; (meth)acrylic acid esters or amides containing a linear, branched, or cyclic C1-C24 alkyl group, a C6-C24 aryl group or a C2-C24 hydroxyalkyl group. These polymers may be obtained from monomers such as isooctyl(meth)acrylate, isononyl(meth)acrylate, 2-ethylhexyl(meth)acrylate, lauryl(meth)acrylate, isopentyl(meth)acrylate, n-butyl(meth)acrylate, isobutyl(meth)acrylate, ethyl(meth)acrylate, methyl(meth)acrylate, tert-butyl(meth)acrylate, tridecyl(meth)acrylate, stearyl(meth)acrylate, hydroxyethyl(meth)acrylate, 2-hydroxypropyl(meth)acrylate, benzyl acrylate, phenyl acrylate, and mixtures thereof. Amides monomers include but are not limited to (meth)acrylamides, such as N-alkyl(meth)acrylamides, for example of a C2-C12 alkyl, such as N-ethylacrylamide, N-t-butylacrylamide, and N-octylacrylamide; N-di(C1-C4)alkyl (meth)acrylamides and perfluoroalkyl(meth)acrylates.

Organic polymers may also include embodiments based upon attachment of a vinyl group to a diverse number of compounds. Polymerization delivers the polyvinyl compound (e.g., a version of polyolefins) with a large variation of substituent identity. Examples of vinyl monomers for such polymerization include but are not limited to vinyl alkanoate such as vinyl acetate, N-vinylpyrrolidone, vinylcaprolactam, vinyl N—(C1-C6)alkylpyrroles, vinyloxazoles, vinylthiazoles, vinylpyrimidines, vinyl pyridine, vinyl thiophene, and vinylimidazoles, olefins such as ethylene, propylene, butenes, isoprene, and butadienes.

Organic polymers also include but are not limited to, for example, of the alkyl acrylate/cycloalkyl acrylate copolymer, the acrylates/C12-22 alkyl methacrylate copolymer and vinylpyrrolidone copolymers, such as copolymers of a C2-C30 alkene, such as a C3-C22 alkene, and combinations thereof. VP copolymers include but are not limited to VP/vinyl laurate copolymer, the VP/vinyl stearate copolymer, the butylated polyvinylpyrrolidone (PVP) copolymer, the VP/hexadecene copolymer, the VP/eicosene copolymer, the VP/triacontene copolymer or the VP/acrylic acid/lauryl methacrylate copolymer, octylacrylamide/acrylates/butylaminoethyl methacrylate copolymer, acrylates/octylacrylamide copolymer, polymers bearing fluoro groups belonging to one of the classes described in the above text, and the copolymers of alkyl(meth)acrylate and perfluoroalkyl(meth)acrylate. Additional precursor organic polymers include those resulting from the polymerization or copolymerization of an ethylenic monomer, comprising at least one ethylenic bond, which can be, for example, conjugated (or dienes). Precursor organic polymer resulting from the polymerization or copolymerization of an ethylenic monomer, vinyl, acrylic, or methacrylic copolymers are also included without limitation.

Organic polymers as block copolymers are also included, examples of which include but are not limited to a block copolymer comprising at least one block comprising styrene units or styrene derivatives (for example methylstyrene, chlorostyrene, or chloromethylstyrene). The copolymer comprising at least one styrene block may also comprise, for example, an alkylstyrene (AS) block, an ethylene/butylene (EB) block, an ethylene/propylene (EP) block, a butadiene (B) block, an isoprene (I) block, an acrylate (A) block, or a methacrylate (MA) block, or a combination of these blocks. The copolymer comprising at least one block of styrene units or styrene derivatives may be a diblock or triblock copolymer, for example of the polystyrene/polyisoprene or polystyrene/polybutadiene type, those of the polystyrene/copoly(ethylene-propylene) type or alternatively of the polystyrene/copoly(ethylene/butylene) type as well as styrene-methacrylate copolymers.

Further embodiments of organic polymers include but are not limited to those chosen from copolymers of vinyl ester (the vinyl group being directly connected to the oxygen atom of the ester group and the vinyl ester having a saturated, linear or branched hydrocarbon-based radical of 1 to 19 carbon atoms bonded to the carbonyl of the ester group) and of at least one other monomer chosen from vinyl esters (other than the vinyl ester already present), α-olefins (containing from 8 to 28 carbon atoms), alkyl vinyl ethers (in which the alkyl group contains from 2 to 18 carbon atoms), or allylic or methallylic esters (containing a linear or branched saturated hydrocarbon-based radical of 1 to 19 carbon atoms, bonded to the carbonyl of the ester group).

Further non-limiting examples of the organic polymers include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, vinyl dimethylpropionate/vinyllaurate, vinyl acetate/octadecyl vinyl ether, vinyl acetate/allyl stearate, vinyl acetate/1-octadecene and allyl propionate/allyl stearate.

Organic polymers also include but are not limited to polycondensates which include but are not limited to polyurethanes, polyurethane-acrylics, polyurethane-polyvinylpyrrolidones, polyester-polyurethanes, polyether-polyurethanes, polyureas, polyurea-polyurethanes, and mixtures thereof. The precursor polyurethanes may be, for example, a copolymer of aliphatic, cycloaliphatic, or aromatic polyurethane, or of polyurea-polyurethane.

The polyurethanes may also be obtained from branched or unbranched polyesters or from alkyds comprising mobile hydrogens that are modified via a polyaddition with a diisocyanate and an organic difunctional (for example dihydro, diamino or hydroxy-amino) coreagent.

Non-limiting examples of organic polymer may also include polyesters, polyester amides, fatty-chain polyesters, polyamides, and epoxyester resins. The precursor polyesters may be obtained in a known manner via the polycondensation of aliphatic or aromatic diacids with aliphatic or aromatic diols or with polyols. Succinic acid, glutaric acid, adipic acid, pimelic acid, suberic acid, or sebacic acid may be used as aliphatic diacids. Terephthalic acid or isophthalic acid, or even a derivative such as phthalic anhydride, may be used as aromatic diacids. Ethylene glycol, propylene glycol, diethylene glycol, neopentyl glycol, cyclohexanedimethanol, and 4,4-N-(1-methylpropylidene)bisphenol may be used as aliphatic diols.

The polyesteramides may be obtained in a manner similar to that for the polyesters, via the polycondensation of diacids with amino alcohols. The polyamides may be obtained in a manner similar to that for the polyesters, via the polycondensation of diacids with diamines. Exemplary precursor polyesters that may be mentioned include aliphatic polyesters containing C4-50 alkyl side chains or polyesters resulting from the condensation of fatty acid dimers, or alternatively polyesters comprising a silicone segment in the form of a terminal block, graft, or group.

### B The Crosslinker

The term “crosslinker” as used herein refers to compounds able to initiate crosslinking of functional groups present on the at least one bio-based polymer, present on any polymer other than the bio-based polymer(s), if present, and/or present on the keratin fibers. In particular, crosslinkers may catalyze or initiate the crosslinking by promoting the formation of covalent bonds, hydrogen bonding, electrostatic or ionic interaction, ionic gelation, etc.

### B1 the Crosslinker Initiating Free-Radical Polymerization

According to embodiments, the crosslinker comprises a compound suitable to induce or promote free-radical polymerization. Examples of crosslinkers able to catalyze crosslinking are compounds such as photoinitiators and thermal crosslinkers, and other compounds able to initialize free-radical polymerization. Photoinitiators generally encompass compounds which decompose to reactive species, i.e. free radicals when exposed to radiation. Suitable photoinitiators typically absorb radiation having a wavelength in the visible or ultraviolet range, for example in the visible or near-UV range, more specifically in the range of 300-500 nm, such as 350-430 nm. Preferred photoinitiators include for example compounds commercially available under the tradename Irgacure 2959, or lithium phenyl-2,4,6-trimethylbenzoylphosphinate (LAP).

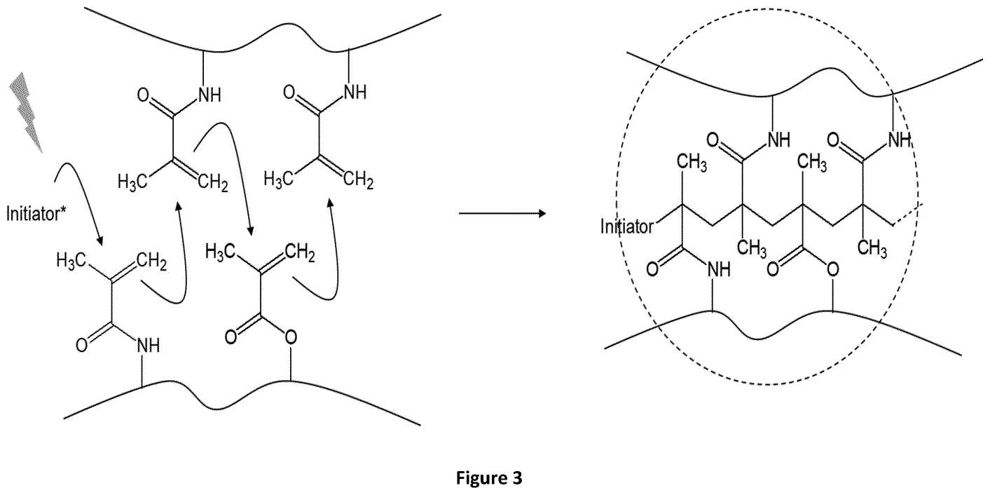

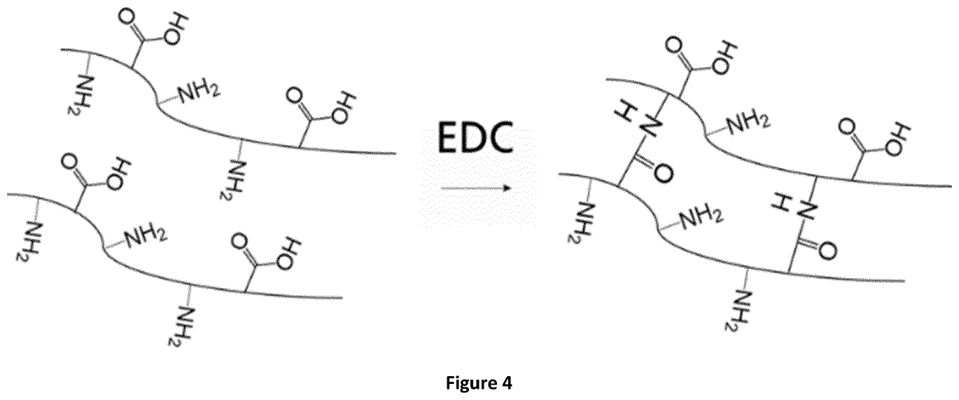

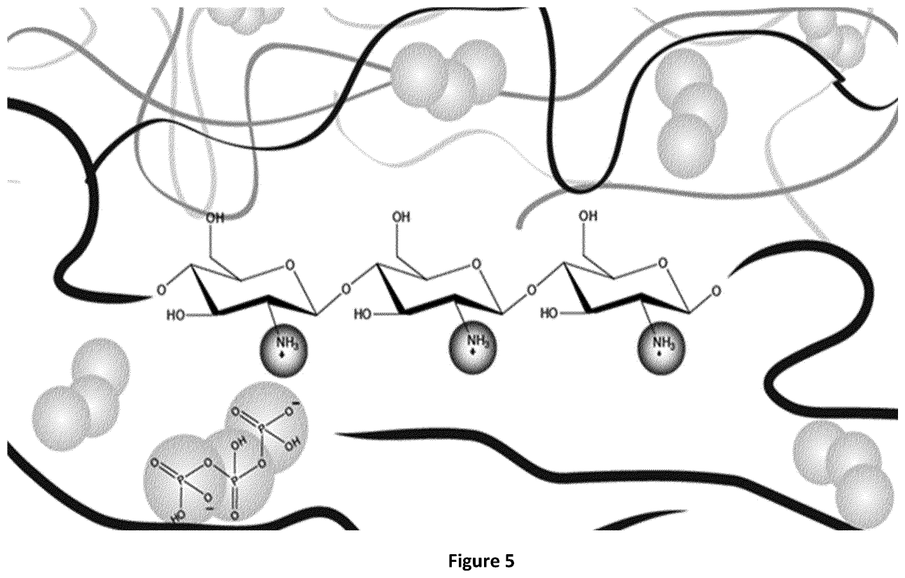

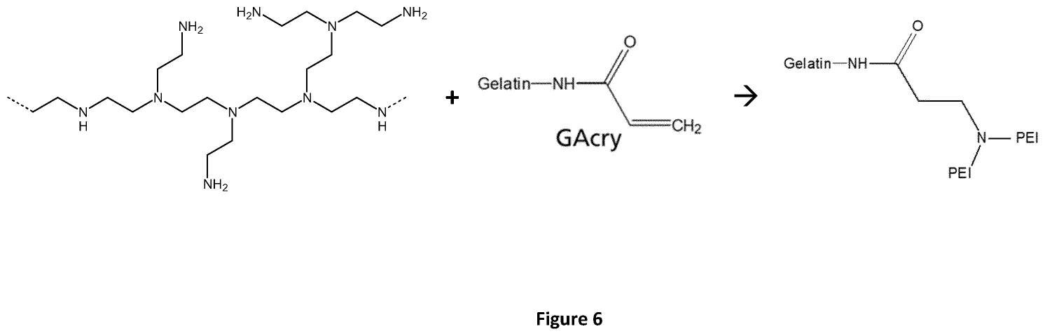

As shown for example in FIG. 3, according to a specific embodiment of the present invention a bio-based polymer modified with methacryloyl groups may crosslink via a crosslinker initializing free-radical polymerization. For example, crosslinking may be induced by exposure to UV radiation in the presence of a photoinitiator.

### B2 the Crosslinker Providing for Ionic Gelation

Crosslinkers suitable for promoting ionic gelation provide electrical charges complementary to and interacting with electrical charges provided by functional groups of particular polymer(s), which electrostatic interaction promotes immobilizing of an entangled matrix comprising the particular polymer(s) and crosslinkers on the keratin fibers. Examples of crosslinkers promoting ionic gelation include for instance polyvalent ions. A specific example of a pair of a crosslinker promoting ionic gelation and a particular polymer is Ca²⁺ ions and polyalginate. Another specific example of a pair of a crosslinker promoting ionic gelation and a particular polymer is polyphosphate anions and chitosan.

### B3 the Crosslinker Having Complementary Functional Groups

Crosslinkers comprising functional groups complementary to functional groups of the polymer(s) generally are compounds able to react with functional groups present on the at least one bio-based polymer, present on any polymer other than the bio-based polymer(s), if present, and/or present on the keratin fibers. The crosslinkers with complementary functional groups may comprise one type of a specific functional group, or more types of specific functional groups. According to embodiments, the crosslinkers with complementary functional groups comprise two types of specific functional groups. Furthermore, the number of functional groups present on each individual crosslinker molecule is at least two, preferably at least three, at least four, at least five, or at least six functional groups are able to react with functional groups of the polymer(s) (optionally with functional groups present on the keratin fibers). According to embodiments, the number of functional groups present on each individual crosslinker molecule may be at least ten, at least fifteen, or at least twenty functional groups able to react with functional groups of the polymer(s) (optionally with functional groups present on the keratin fibers). In view of the crosslinking reaction involving functional groups of the polymer(s) and optionally of the keratin fibers, the crosslinkers comprising complementary functional groups will become integrated into the crosslinked polymer structure. A particular crosslinker may comprise one type of functional groups, or more than one type of functional groups, such as hydroxy, amine, carboxy, mercapto, isocyanate, or olefinoyl. In particular embodiments of the crosslinkers comprising complementary functional groups, the functional groups are selected for example from amine, mercapto, isocyanate and combinations thereof. In specific embodiments, the crosslinkers comprising complementary functional groups may be selected from polythiols, polyisocyanates, and polyethyleneimines. It should be noted in that context that due to toxicity and regulations, (poly)isocyanates may not be used for treating for example keratin fibers (such as scalp) of humans or animals, but might be used for treating keratin fibers on non-living objects.

The crosslinker having complementary functional groups may be a small molecule, a dimer, trimer, tetramer, pentamer, hexamer, oligomer, or small polymer.

One preferred embodiment of the crosslinker is an amine polymer comprising at least two amine-functional groups per polymer chain, wherein the amino functional groups are selected from the group consisting of primary, secondary, tertiary amine-functional groups and mixtures thereof. Embodiments of the crosslinker may be selected from the group consisting of polyethyleneimine, polyallylamine, polyvinylamine, aminopolysaccharides, copolymers thereof and mixtures thereof.

Additional crosslinkers include polythiols. Polythiols suitable as crosslinkers in the present invention typically are molecules having a molecular weight in the range of 200-4,000 g/mol and thio-functionality of at least 2, typically 3-10, for example 3-6. Preferred polythiol crosslinkers are molecules having a molecular weight in the range of 350-1,500 g/mol, such as 480-1,350 g/mol and thio-functionality of 3-6. Examples of polythiol crosslinkers include tri-mercapto and tetra-mercapto branched alkyl compounds wherein the mercapto groups are the termini and the branches are C3-C10 methylenyl groups on a C3-C10 polymethylenyl backbone. Specific examples of crosslinkers include for instance Thiocure® PETMP, Thiocure® TMPMP, Thiocure® GDMP, Thiocure® PETMA, Thiocure® TMPMA, Thiocure® GDMA, Thiocure® ETTMP 1300, Thiocure® ETTMP 700, Thiocure® TEMPIC, Thiocure® Di-PETMP, and Thiocure® PCL4MP. Additional polythiols that might conceivably be used are so called Thiokols which are available and may suitably used at much higher molecular weights. Specific examples of Thiokols are THIOKOL LP manufactured by Toray, POLYTHIOL™ QE-340M manufactured by Toray and THIOKOL® Sealant Systems from PolySpec.

Exemplary polyamine crosslinker include, for example:
a) Linear polyethyleneimine of the formula:

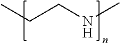

in which n is an integer representing the degree of polymerization, wherein n ranges from 100 to 3,500, alternatively from 200 to 2,000;
b) Branched polyethyleneimine consisting of primary, secondary and tertiary amine groups of the formula:

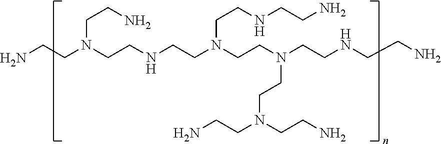

in which n is an integer representing the degree of polymerization, wherein n ranges from 5 to 4,000, alternatively from 50 to 500.

Polyethylene imine crosslinkers suitable in the present invention typically have a weight average molecular weight from about 3,000 g/mol to about 100,000 g/mol, for example from about 5,000 g/mol to about 80,000 g/mol such as from about 10,000 g/mol to about 70,000 g/mol.

Isocyanates suitable as crosslinkers in the present invention include small molecules having 2 or 3 isocyanates groups, such as toluene-2,4-diisocyanate, toluene-2,6-diisocyanate, commercial mixtures of toluene-2,4 and 2,6-diisocyanates, isophorone diisocyanate, ethylene dii socyanate, ethylidene dii socyanate, propylene-1,2-dii socyanate, cyclohexyl ene-1,2-dii socyanate, cyclohexylene-1,4-diisocyanate, m-phenylene diisocyanate, 4,4′-biphenylene diisocyanate, 4,4′-diphenylmethane diisocyanate, 1,6-hexamethylene diisocyanate, 1,4-tetramethylene diisocyanate, 1,10-decamethylene diisocyanate, cumene-2,4-diisocyanate, methylene bis(cyclohexyl isocyanate), p-phenylene diisocyanate, 2,4,6-toluene triisocyanate, and p,p′,p″-triphenylmethane triisocyanate. Further isocyanates suitable as crosslinkers include the reaction products of diisocyanates or triisocyanates with polyols or polyamines, which reaction products typically comprise 2-10 such as 4-6 iosocyanate groups, and a molecular weight in the range of 120-2,000 g/mol. As already noted above herein, diisocyanates, triisocyanates and polyisocyanates may not be used for treating for example keratin fibers (such as scalp) of humans or animals, but might be used for treating keratin fibers on non-living objects

According to embodiments, the functional groups of the crosslinker may be selected from isocyanate, mercapto, amino, olefinoyl, or a combination thereof, and the functional groups of the at least one bio-based polymer may be selected from olefinoyl, carboxy, amino, mercapto, or a combination thereof. According to specific embodiments, the at least one bio-based polymer is selected from proteins, olefinoyl-modified proteins, polysaccharides, carboxy-functional polysaccharides, amino-functional polysaccharides, olefinoyl-modified polysaccharides, and combinations thereof, and the crosslinker comprises isocyanate-functional groups. According to other specific embodiments, the at least one bio-based polymer comprises olefinoyl-functional groups, and the crosslinker comprises amine-functional groups and/or mercapto-functional groups.

### B4 Other Crosslinkers

According to further embodiments, the crosslinkers induce the crosslinking of functional groups without becoming integrated into the crosslinked polymeric structure. Examples of such crosslinkers include for example carbodiimides. Carbodiimides are able to react with carboxy-functional groups present on the polymer(s) or present on the keratin fibers via formation of an unstable o-acylisourea intermediate, which intermediate subsequently may react with a primary amine group, thereby forming an amide bond. In this latter reaction step, the remnant of the carbodiimide moiety represents a leaving group, which does not become integrated into the crosslinked polymeric structure. A typical example of a carbodiimide suitable as an inducing crosslinker is 1-ethyl-3-(3-dimethylaminopropyl)carbodiimide (EDC).

Crosslinkers such as EDC may be used for crosslinking one or more types of bio-based polymers comprising both carboxy functional groups and amine functional groups. Similarly, crosslinkers such as EDC may be used for crosslinking two or more types of bio-based polymers wherein at least one bio-based polymer comprises carboxy functional groups and at least one bio-based polymer comprises amine functional groups. In addition to provide for crosslinking of functional groups present on one or more types of bio-based polymers, crosslinkers such as EDC may also provide for crosslinking between functional groups present on bio-based polymers and functional groups present on the keratin fibers.

Other suitable crosslinkers for the bio-based polymers are monomeric, oligomeric, polymeric silanes including but not limited to aminosilanes, epoxysilanes, isocyanate silanes, alkylsilane, alkoxysilane, chlorosilane, ureidosilane, phenylsilane, vinylsilane, alkyl silicate, methacrylatesilane, methacryloxysilane, mercaptosilane, 4-Ethyl-2,3-dioxo-1-piperazine carbonyl chloride(EDPC), N-(Trimethylsilyl)imidazole (TSIM), Hexamethyldisilazane (HMDS), Hexamethyldisiloxane (HMDO), N,O-Bis(trimethylsilyl)acetamide (BSA), N,O-Bis(trimethylsilyl)trifluoroacetamide (BSTFA), N,N′-Thiocarbonyldiimidazole(TCDI). The said silanes can be used to separately crosslink the bio-based polymers or can be incorporated as additives in the end-use formulation to provide a crosslinking function in-situ at the point of use. The said silanes can contain one or more (preferably more than 1) reactive group.

Another suitable crosslinker for the bio-based polymers is genipin (methyl (1R,2R,6S)-2-hydroxy-9-(hydroxymethyl)-3-oxabicyclo[4.3.0]nona-4,8-diene-5-carboxylate). Genipin is a natural molecule that may be obtained from fruit extract of Genipa Americana. It is a crosslinker having low acute toxicity.

### C Functional Groups of Polymers and Crosslinkers

Crosslinking may be accomplished by reaction between pairs of complementary functional groups present on the bio-based polymer(s), polymers other than bio-based polymers, and crosslinkers form complementary reactive pairs of functional groups according to the following pairs. Complementary functional groups on the keratin fibers also may contribute to the crosslinking. Such pairs of complementary functional groups include:
i) isocyanate or thioisocyanate and hydroxyl, amine or mercapto or any combination of hydroxyl, amine and mercapto;
ii) carboxyl and hydroxyl, amine or mercapto or any combination of hydroxyl, amine and mercapto;
iii) alkylepoxy and hydroxyl, amine or mercapto or any combination of hydroxyl, amine and mercapto;
iv) olefinoyl and hydroxyl, amine or mercapto or any combination of hydroxyl, amine and mercapto (an example of olefinoyl is acryloyl or crotonyl);
v) olefinoyl and furanyl or pentadienyl or a combination of furanyl and pentadienyl;
vi) malonic anhydrido and hydroxyl, amine or mercapto or any combination of hydroxyl, amine and mercapto;
vii) formyl and amine or mercapto or any combination of amine and mercapto;
viii) vinyl and amine or mercapto or a combination of amine and mercapto;
ix) vinyl and furanyl or pentadienyl or a combination of furanyl and pentadienyl;
x) azido and alkynyl;
xi) when the functional groups are a self-reactive functional group, mercapto and mercapto to form disulfide; and,
xii) when the functional groups are a self-reactive functional group, olefinoyl and olefinoyl, to form for example a structure such as shown in FIG. 3.

Preferred reactive pairs of complementary functional groups include:
i) isocyanate and hydroxyl, amine or mercapto or any combination of hydroxyl, amine and mercapto;
ii) epoxy and hydroxyl, amine, or mercapto or any combination any two or more of hydroxyl, amine and mercapto;
iii) olefinoyl and hydroxyl, amine or mercapto or any combination of any two or more of hydroxyl and amine and mercapto, and a preferred embodiment of the olefinoyl group is (meth)acryloyl or crotonyl;
iii) carboxyl and hydroxyl, amine or mercapto or any combination of any two or more of hydroxyl, amine and mercapto;
iv) mercapto and mercapto;
v) olefinoyl and olefinoyl.

Especially preferred reactive pairs of complementary functional groups include:
i) isocyanate and hydroxyl, amine or mercapto or any combination of hydroxyl, amine and mercapto;
ii) carboxyl and hydroxyl or amine or a combination of hydroxyl and amine;
iii) amine and carboxy or isocyanate or (meth)acryloyl;
iv) mercapto and isocyanate or (meth)acryloyl; and
v) (meth)acryloyl and amine or mercapto or hydroxy.

According to embodiments, especially preferred functional groups present on the bio-based polymer(s) are carboxy, amine, mercapto and (meth)acryloyl. According to embodiments, especially preferred functional groups present on the crosslinker(s) are amine, mercapto and (meth)acryloyl.

### D The Pigment Microparticle

The color composition embodiments of the present invention make it possible to obtain colored and remnant coatings, without substantially altering the keratin fibers. As used herein, the term “pigment” generally refers to any particle colorant having or containing pigment material that gives hair fibers color including black and white, such as titanium dioxide that give only white to hair fibers. The pigments, to distinguish from dyes presented in molecular from, are also referred to as pigment microparticles or pigment particles. The terms pigment microparticles and pigment particles are synonymous and are used herein interchangeably. The pigments can be organic, inorganic, or a combination of both. The pigments may be in pure form or coated, for example with a polymer or a dispersant.

Selections, multiple kinds and varying forms of the pigment microparticles as described in the following passages can be incorporated in the composition of the present invention, or can be applied separately to the keratin fibers. Preferably, pigment microparticles can be incorporated into the composition. When applied separately, pigment particles will be applied in the form of a composition, typically an aqueous dispersion.

The at least one pigment that can be used can be chosen from the organic and/or mineral pigments known in the art, such as those described in Kirk-Othmer's Encyclopedia of Chemical Technology and in Ullmann's Encyclopedia of Industrial Chemistry. The pigments comprised in the microparticles comprising at least one pigment will not substantially diffuse or dissolve into keratin fibers. Instead, the pigment comprised in the microparticles comprising at least one pigment will substantially remain separate from but attached to the keratin fibers.

The at least one pigment can be in the form of powder or of pigmentary paste. It can be coated or uncoated. The at least one pigment can be chosen, for example, from mineral pigments, organic pigments, elemental metal and their oxides, and other metal modifications, lakes, pigments with special effects such as nacres or glitter flakes, and mixtures thereof.

D1 Pigment shape

The pigment microparticles can have any suitable shape, including substantially spherical. But the pigment microparticles can also be oval, elliptical, tubular, irregular, etc., or even combinations of various shapes. In addition, the pigment microparticles can have two dimensions, length and width/diameter, of similar magnitude. In addition, the pigment microparticles can be micro platelets, i.e. having a thickness that is substantially smaller than the planar dimension. For example, five, ten or even 20 times smaller in thickness than in the planer dimension. In one embodiment with any of the reactive components of the instant invention, the pigments may be surface treated, surface coated or encapsulated.

In a particular aspect, the pigment microparticles can have a shape approximating that of a sphere, in which case the microparticles are referred to as being microspheres. Pigment microparticles which can be described as microspheres are understood as particles having an aspect ratio, defined as a function of the largest diameter, or largest dimension, dmax and the smallest diameter, or smallest dimension, dmin, which can be orthogonal to each other: AR=dmax/dmin which is from about 1:1 to 10:1, preferably from 1:1 to 5:1, more preferably from 1:1 to 4:1, such as from 1:1 to 3:1. More particularly, the expression “spherical-type” means that the pigment microparticles have a shape approximating that of a sphere. In other words, the pigment microparticles can be nearly orbicular in shape and can have a cross-sectional geometry that is essentially circular. Although not excluded, this does not necessarily mean that the pigment microparticles have the shape of a perfect sphere or ball. More likely, the shape of the pigment microparticles can exhibit a certain deviation from a sphere as long as the skilled person considers the shape as being similar to a sphere or as an approximation of a sphere.

In addition, the pigment microparticles can have a rather two-dimensional shape, with the smallest dimension substantially smaller than the two other dimensions, in which case the microparticles are referred to as being 2-dimensional microparticles. For example, the thickness of the microparticles can be significantly less than their length and width. The length and width can be of similar magnitude. Examples includes pigment microparticles having a shape of platelets, i.e. with a thickness that is substantially smaller than the planar dimension. For example, the aspect ratio AR=dmax/dmin, as defined above, of microparticles having a substantially two-dimensional shape, can be from about 10:1 to about 1000:1, preferably from about 10:1 to about 800:1, preferably from about 20:1 to about 800:1, preferably from about 10:1 to about 600:1, preferably from about 20:1 to about 600:1. Typically, the 2D-microparticles have a largest and a smallest dimension in their planer dimension, which both are significantly larger than the smallest dimension of the 2D-microparticles extending perpendicular to the planer dimension.

According to an embodiment, the pigments can include pigment microparticles of different shape. For example, microparticles of different size can be used to provide different reflecting and absorbing properties. Microparticles having different shape can also be formed of different pigment material. Furthermore, microparticles having different shape can also formed of different pigment material to provide different color.

### D2 Pigment Size

The pigments can be present in the composition in undissolved form. Depending on the shape, the pigments can have a D50[vol] particle diameter of from 0.001 micron to 1 micron.

For example, pigments that can be described as being microspheres can have a D50[vol] particle diameter of from 0.01 micron to 1 micron, preferably of from 0.015 micron to 0.75 micron, more preferably of from 0.02 micron to 0.50 micron. The microspheres can also have a D50[vol] particle diameter of from 0.6 micron to 0.9 micron, preferably of from 0.08 micron to 0.9 micron, and more preferably between of from 0.08 micron to 0.9 micron, such as from 0.08 micron to 0.8 micron, or such as of from 0.8 micron to 0.6 micron. According to an embodiment, the microspheres can also have a D50[vol] particle diameter of from 0.1 micron to 1 micron, preferably of from 0.12 micron to 1 micron, and more preferably between of from 0.16 micron to 1 micron, such as of from 0.2 micron to 1 micron, or such as of from 0.08 micron to 0.4 micron. The terms “micron” and “microns” describe the size in micrometers [μm].

In further embodiments, which can be combined with other embodiments described herein, the pigments, which can be described as microspheres, can have a D90[vol] particle diameter of from 0.1 micron to 1 micron, preferably of from 0.2 micron to 1 micron, and more preferably between of from 0.3 micron to 1 micron, such as of from 0.3 micron to 0.9 micron, or such as of from 0.4 micron to 0.8 micron, or such as of from 0.5 micron to 0.9 micron.

In some embodiments described herein, the pigments, which can be described as microspheres, can have a D10[vol] particle diameter of from 0.02 micron to 0.3 micron, preferably of from 0.06 micron to 0.3 micron, more preferably of from 0.08 micron to 0.3 micron, such as of from 0.08 micron to 0.2 micron, or such as of from 0.1 micron to 0.2 micron, or such as 0.12 micron to 0.3 micron.

In embodiments described herein, the D10[vol] particle diameter can be of from 0.02 micron to 0.3 micron and the D90[vol] can be of from 0.3 micron to 1 micron. In further embodiments, the D10[vol] particle diameter can be of from 0.06 micron to 0.2 micron and the D90[vol] can be of from 0.4 micron to 1 micron.

The particle diameter is represented by D10, D50 and/or by D90, which is the median diameter by volume. D10, D50 and D90 is measured with a Malvern Mastersizer 2000, which is a laser diffraction particle sizer and it is measured according to ISO 13320:2009(en) with Hydro 2000G or Hydro 2000S where the dispersant is water or ethanol. Detection range is from 0.01 micron to 2000 micron. D50 is expressed as x50 in ISO 13320:2009(en).

The term “D10,” as used herein refers, to the 10th percentile number- or volume-based median particle diameter, which is the diameter below which 10% by number or volume of the particle population is found. The term “D50,” as used herein refers, to the 50th percentile number -or volume-based median particle diameter, which is the diameter below which 50% by number or volume of the particle population is found. The term “D90,” as used herein refers, to the 90th percentile number- or volume-based median particle diameter, which is the diameter below which 90% by number or volume of the particle population is found. The number or volume measurement is indicated by [num] for number or [vol] for volume. If not indicated otherwise, the particle size is given as D10[vol], D50[vol], and D90[vol], respectively.

Laser diffraction measures particle size distributions by measuring the angular variation in intensity of light scattered as a laser beam passes through a dispersed particulate sample analyzer and the particle size is reported as a volume equivalent sphere diameter. A discussion of calculating D50 is provided in Barber et al, Pharmaceutical Development and Technology, 3(2), 153-161 (1998), which is incorporated herein by reference. Pigment microparticles having a D50[vol] particle diameter of less than 20 nm may enter the cuticles and are therefore difficult to remove. For scattering purposes, Pigment(s) having a D10[vol] particle diameter of at least 60 nm, or at least 80 nm can be used. Pigment(s) having a D50[vol] particle diameter of more than 1 micron typically do not sufficiently adhere onto hair fibers.

According to an embodiment, the particle size distribution, either relative to the number or volume of the particles, of the pigment microparticles can be at least bi-modal. A bi-modal particle size distribution has two distinct peaks which are spaced relative from, while tri-modal particle size distribution has three distinct peaks. The term “peak” means a local maximum of the distribution curve. The “distance” between two peaks, expressed relative to the particle size, can be at least 0.05 micron, preferably at least 0.1 micron, such as at least 0.2 micron. Providing an at least bi-modal particle size distribution allows to tailor the optical appearance of the colored hair. For example, the scattering properties varies with the particle size so that particles of different size scatter the light into different directions.

The at least bi-modal particle size distribution can be relative to pigment microparticles formed by the same pigment material. In addition to that or alternatively, the at least bi-model particle size distribution can be provided by pigment microparticles of different pigment material.

The size of pigment microparticles which can be described to have a 2-dimensional shape, and which are referred to as 2-dimensional microparticles can be determined by SEM. The size of 2-dimensional microparticles can also be determined by laser diffraction measurements. The particle size determined by laser diffraction is a mean size of the different dimensions of the 2-dimensional particles. The apparent D50[vol] particle diameter of 2-dimensional microparticles, as measured by SEM, can be from 0.5 micron to 50 microns, more preferably from 0.8 micron to 20 microns, more preferably from 1 micron to 15 microns, more preferably from 1.5 micron to 10 microns.

According to an embodiment, pigment particles are referred to as being microspheres can be used light-scattering and/or light absorbing purposes. Those particles, due to their pigment material, impart the hair with a specific color.

According to an embodiment, pigment particles are referred to as being 2-dimensional microparticles can be mainly used for light-reflecting and/or light absorbing purposes. Those particles, due to their pigment material, mainly reflect the light without significantly alter the color of the light.

The pigment microparticles can be light absorbing, but which for wavelengths of visible light provide negligible to low or no scattering. While not wishing to bound by any specific theory, it is believed that such pigments can provide more chromatic colors. Such pigment microparticles can have a D50[vol] value between about 0.001 micron and about 0.15 micron, between about 0.005 micron and about 0.1 micron or between about 0.010 micron and about 0.075 micron.

The pigment microparticles can be predominantly light scattering for wavelengths of visible light and provide low light absorption. While not wishing to bound by any specific theory, it is believed that such pigments can provide the visual effect of lightening the hair. Such pigment microparticles, which can be microspheres, can have a D50[vol] value between about 0.05 micron to about 1 micron, between 0.08 micron to about 0.9 micron, between about 0.05 micron and about 0.75 micron, between about 0.1 micron and about 0.5 micron or about 0.15 micron and about 0.4 micron. Such materials can have a refractive index above 1.5, above 1.7 or above 2.0.

Pigments made from metal and metal like materials which can conduct electricity, and which can absorb light and re-emit the light out of the metal to give the appearance of strong reflectance. While not wishing to be bound by any specific theory, it is believed that the absorbed light will induce alternating electric currents on the metal surface, and that this currents immediately re-emit light out of the metal. Such pigment microparticles can be platelets, e.g., having a thickness that is substantially smaller than the planar dimension. For example about five, about 10 or even about 400 times smaller in thickness than in the planer. Such platelets can have a planar dimension less than about 30 nm, but with a thickness less than about 10 micron wide. This includes a ratio of 10000 to 30, or 333. Platelets larger in size, such as 50 microns are even available in this thickness of 10 microns, and so the ratios can even go up to 2000.

The pigment microparticles can be a composite formed by two different types of pigment microparticles. Examples include a composite of a 2-dimensional microparticle and at least one micro spherical particle (microsphere), a composite of different micro spherical particles, and a composite of different 2-dimensional particles. Composite particles formed by 2-dimensional microparticles to which micro spherical particles adhere provide an attractive alternative to a pure mixture of 2-dimensional microparticles and micro spherical particles. For example, a metallic 2-dimensional microparticle can carry one or more micro spherical particle such as one or more organic micro spherical particle. The micro spherical particles attached or bonded to the 2-dimensional microparticle can be formed of the same pigment material or can be formed of different pigment material. Composite microparticles formed of 2-dimensional microparticles and micro spherical particles can provide multiple functionality in one particle such as (metallic) reflectance and dielectric scattering, reflectance and absorption.

Pigment microparticles may be materials which are composite comprising a core of pigments made from metal and metal like materials which can conduct electricity, and which can absorb light and re-emit the light out of the metal to give the appearance of strong reflectance. While not wishing to be bound by any specific theory, it is believed that the absorbed light will induce alternating electric currents on the metal surface, and that this currents immediately re-emit light out of the metal. Upon this pigment light absorbing microparticles is immobilized. Such pigment microparticles can be platelets, e.g., having a thickness that is substantially smaller than the planar dimension. For example, five, ten or even 20 times smaller in thickness than in the planer. Such platelets can have a planer dimension less than 15 microns, but with a thickness less than 1 microns, more preferably with a planer dimension less than 12 microns but with a thickness less than 750 nm, even more preferably with a plan dimension less than 10 microns and a thickness less than 0.5 micron. The light absorbing microparticles can have D50 [vol] value between 0.001 micron and 0.15 micron, more preferably between 0.002 micron and 0.1 micron and even more preferable between 0.005 micron and 0.075 micron.

The light absorbing microparticles may also include dyes, pigments, or materials with color centers in the crystal structure, or photonic structures resulting in destructive or constructive interference, diffraction or other structures and materials mentioned in the book “The Physics and Chemistry of Color: the Fifteen Causes of Color”, 2nd Edition by K. I. Nassau (ISBN 978-0-471-39106-7).

The pigment microparticles can be both light scattering and absorbing for wavelengths of visible light. While not wishing to bound by any specific theory, it is believed that such pigments can provide both some visual effect of lightening the hair. Such pigment microparticles can have a D50[num] value between about 50 nm and about 750 nm, between about 100 nm and about 500 nm or between about 150 nm and about 400 nm. Such materials have a refractive index above about 1.5, above about 1.7 or above about 2.0.

According to an embodiment, different pigment microparticles are combined to provide reflective, transmitting and refractive properties of the hair colored with the color composition described herein. A microparticle combination can be a material composite using at least two different pigment materials to form the pigment microparticles. In addition to, or alternating to, the microparticle combination, a mixture of separate pigment microparticles of different type can be used to bring about the desired reflective, transmitting and refractive properties.

The composite pigments, combination of pigments, and mixtures of pigment microparticles eliminate, or at least significantly reduce, hair penetration and scattering by light and thus eliminate the perception of pigment of natural hair color change.

### D3 Pigment Concentration

The composition for coloring keratin fibers such as hair fibers according to the present disclosure comprises microparticles comprising at least one pigment. The color composition comprises from about 0.01% to about 40%, about 0.05% to about 35%, about 0.1 to about 25%, or about 0.15% and about 20% pigment(s), by weight of the color composition.

### D4 Pigment Material

The material of the pigment microparticles can be inorganic or organic. Inorganic-organic mixed pigments are also possible.

According to an embodiment, inorganic pigment(s) are used. The advantage of inorganic pigment(s) is their excellent resistance to light, weather, and temperature. The inorganic pigment(s) can be of natural origin, and are, for example, derived from material selected from the group consisting of chalk, ochre, umber, green earth, burnt sienna, and graphite. The pigment(s) can preferably be white pigments, such as, for example, titanium dioxide or zinc oxide. The pigment(s) can also be colored pigments, such as, for example, ultramarine or iron oxide red, luster pigments, metal effect pigments, pearlescent pigments, and fluorescent or phosphorescent pigments. The pigment(s) can be selected from the group consisting of metal oxides, hydroxides and oxide hydrates, mixed phase pigments, sulfur-containing silicates, metal sulfides, complex metal cyanides, metal sulfates, chromates and molybdates, alloys, and the metals themselves. The pigment(s) can be selected from the group consisting of titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal), zinc sulfide, barium sulfate, zinc oxide, siliconised titanium dioxide, siliconised zinc sulfide, siliconised zinc oxide, and mixtures thereof. The pigment(s) can be selected from the group consisting of iron oxide, titanium dioxide, mica, borosilicate, and combinations thereof. The pigment(s) can comprise an iron oxide (Fe₂O₃) pigment. The pigment(s) can comprise a combination of mica and titanium dioxide.

The pigment(s) can be pearlescent and colored pigment(s), and can preferably be based on mica which are coated with a metal oxide or a metal oxychloride, such as titanium dioxide or bismuth oxychloride, and optionally further color-imparting substances, such as iron oxides, Prussian blue, ultramarine, and carmine. The color exhibited by a pigment can be adjusted by varying the layer thickness. Such pigments are sold, for example, under the trade names Rona®, Colorona®, Dichrona®, RonaFlair®, Ronastar®, Xirona® and Timiron® all of which are available from Merck, Darmstadt, Germany. For example, Xirona® is a brand for color travel pigments that display color shifting effects depending on the viewing angle and are based on either natural mica, SiO₂ or calcium aluminum borosilicate flakes, coated with varying layers of TiO₂. Pigment(s) from the line KTZ® from Kobo Products, Inc., 3474 So. Clinton Ave., So. Plainfield, USA, are also useful herein, in particular the Surface Treated KTZ® Pearlescent Pigments from Kobo. Particularly useful are KTZ® FINE WHITE (mica and TiO2) having a D50 particle diameter of 5 to 25 micron and also KTZ® CELESTIAL LUSTER (mica and TiO2, 10 to 60 micron) as well as KTZ® CLASSIC WHITE (mica and TiO2, 10 to 60 micron). Also useful are SynCrystal Sapphire from Eckart Effect Pigments, which is a blue powder comprising platelets of synthetic fluorphlogopite coated with titanium dioxide, ferric ferrocyanide and small amounts of tin oxide. Also useful is SYNCRYSTAL Almond also from Eckart, which is a beige powder with a copper reflection color and is composed of platelets of synthetic fluorphlogopite and coated with titanium dioxide and iron oxides. Also useful is Duocrome® RV 524C from BASF, which provides a two color look via a lustrous red powder with a violet reflection powder due to its composition of mica, titanium dioxide and carmine. The colored pigment(s) can be lightly bright colored pigment(s), and can particularly be white color variations.

The pigment(s) can be organic pigments. The at least one pigment can be an organic pigment. As used herein, the term “organic pigment” means any pigment that satisfies the definition in Ullmann's encyclopedia in the chapter on organic pigments. For instance, the at least one organic pigment can be chosen from nitroso, nitro, azo, xanthene, quinoline, anthraquinone, phthalocyanin, copper phthalocyanin, copper hexadecachlorophthalocyanine, 2-[(2-Methoxy-4-nitrophenyl)azo]-N-(2-methoxyphenyl)-3-oxobutyramide, metal-complex, isoindolinone, isoindoline, quinacridone, perinone, perylene, diketopyrrolopyrrole, thioindigo, dioxazine, triphenylmethane, dimethylquinacridone and quinophthalone compounds, Azo-dyes, Nonionic azo dyes, Anionic Azo dyes, Cationic azo dyes, Complex forming azo dye, aza annulene dyes, aza analogue of diarylmethane dyes, aza annulene dyes, Nitro-dyes and their pigments, Carbonyl dyes and their pigments (for example, Anthrachinon dyes, indigo), Sulfur dyes, Florescence dyes, Anthracene or Insoluble alkali or earth metal acid dyes.

Or the pigment can be at least one of uncolored and UV absorbing.

The organic pigment(s) can be selected from the group consisting of natural pigments sepia, gamboge, bone charcoal, Cassel brown, indigo, chlorophyll and other plant pigments. The synthetic organic pigments can be selected from the group consisting of azo pigments, anthraquinoids, indigoids, dioxazine, quinacridone, phthalocyanine, isoindolinone, perylene and perinone, metal complex, alkali blue, diketopyrrolopyrrole pigments, and combinations thereof. A particularly preferred pigment is 7-Bis(1,3-dichloropropan-2-yl)benzo[lmn][3,8]phenanthrolin-1,3,6,8(2H,7H)-tetraon.

According to an embodiment, the pigment(s) can be selected from the pigment group consisting of, including any combination thereof (with CI meaning color index and CAS meaning Chemical Abstract Service Number)

Pigment Black 10 [C.I. 77265, (CAS: 7782-42-5)], Pigment Black 11 [C.I. 77499, (CAS: 12227-89-3)], Pigment Black 12 [CI. 77543, (CAS: 68187-02-0)], Pigment Black 13 [C.I. 77322, (CAS: 1307-96-6)], Pigment Black 14 [C.I. 77728, (CAS: 83512-98-5)], Pigment Black 15 [C.I. 77403, (CAS: 1317-38-0)], Pigment Black 17 [C.I. 77975, (CAS: 1314-98-3)], Pigment Black 18 [C.I. 77011, (CAS: 12001-98-8)], Pigment Black 23 [C.I. 77865, (CAS: 68187-54-2)], Pigment Black 24 [C.I. 77898, (CAS: 68187-00-8)], Pigment Black 25 [C.I. 77332, (CAS: 68186-89-0)], Pigment Black 26 [C.I. 77494, (CAS: 68186-94-7)], Pigment Black 27 [C.I. 77502, (CAS: 68186-97-0)], Pigment Black 28 [C.I. 77428, (CAS: 68186-91-4)], Pigment Black 29 [CI. 77498, (CAS: 68187-50-8)], Pigment Black 30 [C.I. 77504, (CAS: 71631-15-7)], Pigment Black 31 [C.I. 71132, (CAS: 67075-37-0)], Pigment Black 32 [CI. 71133, (CAS: 83524-75-8)], Pigment Black 33 [C.I. 77537, (CAS: 188735-18-4)], Pigment Black 34 [C.I. 77770, (CAS: 1317-33-5)], Pigment Black 6 [C.I. 77266, (CAS: 1333-86-4)], Pigment Black 7 [C.I. 77266, (CAS: 1333-86-4)], Pigment Black 8 [C.I. 77268, (CAS: 1339-82-8)], Pigment Black 9 [C.I. 77267, (CAS: 8021-99-6)], Pigment Blue 10 [CI. 44040, (CAS: 1325-93-5)], Pigment Blue 15 [CI. 74160, (CAS: 147-14-8)], Pigment Blue 16 [CI. 74100, (CAS: 574-93-6)], Pigment Blue 18 [CI. 42770, (CAS: 1324-77-2)], Pigment Blue 21 [CI. 69835, (CAS: 1324-26-1)], Pigment Blue 22 [C.I. 69810, (CAS: 1324-27-2)], Pigment Blue 25 [C.I. 21180, (CAS: 10127-03-4)], Pigment Blue 26 [CI. 21185, (CAS: 5437-88-7)], Pigment Blue 28 [CI. 77346, (CAS: 1345-16-0)], Pigment Blue 29 [CI. 77007, (CAS: 57455-37-5)], Pigment Blue 30 [C.I. 77420, (CAS: 1339-83-9)], Pigment Blue 32 [CI. 77365, (CAS: 69458-70-4)], Pigment Blue 33 [C.I. 77112, (CAS: 8046-59-1)], Pigment Blue 34 [C.I. 77450, (CAS: 1317-40-4)], Pigment Blue 35 [C.I. 77368, (CAS: 83712-59-8)], Pigment Blue 36 [C.I. 77343, (CAS: 68187-11-1)], Pigment Blue 56 [C.I. 42800, (CAS: 6417-46-5)], Pigment Blue 57 [C.I. 42795, (CAS: 5905-38-4)], Pigment Blue 60 [C.I. 69800, (CAS: 81-77-6)], Pigment Blue 61 [C.I. 42765, (CAS: 1324-76-1)], Pigment Blue 62 [C.I. 42595, (CAS: 82338-76-9)], Pigment Blue 63 [C.I. 73015, (CAS: 16521-38-3)], Pigment Blue 64 [C.I. 69825, (CAS: 130-20-1)], Pigment Blue 65 [C.I. 59800, (CAS: 116-71-2)], Pigment Blue 66 [C.I. 73000, (CAS: 482-89-3)], Pigment Blue 71 [C.I. 77998, (CAS: 68186-95-8)], Pigment Blue 72 [C.I. 77347, (CAS: 68186-87-8)], Pigment Blue 73 [C.I. 77364, (CAS: 68187-40-6)], Pigment Blue 74 [C.I. 77366, (CAS: 68412-74-8)], Pigment Blue 75 [C.I. 74160, (CAS: 3317-67-7)], Pigment Blue 76 [CI. 742520, (CAS: 176365-61-0)], Pigment Blue 78 [C.I. 42090, (CAS: 68921-42-6)], Pigment Blue 79 [C.I. 741300, (CAS: 14154-42-8)], Pigment Blue 9 [CI. 42025B, (CAS: 596-42-9)], Pigment Brown 1 [C.I. 12480, (CAS: 6410-40-8)], Pigment Brown 10 [C.I. 77227, (CAS: 12013-69-3)], Pigment Brown 11 [CI. 77495, (CAS: 64294-89-9)], Pigment Brown 2 [C.I. 12071, (CAS: 10279-43-3)], Pigment Brown 22 [C.I. 10407, (CAS: 29398-96-7)], Pigment Brown 23 [C.I. 20060, (CAS: 35869-64-8)], Pigment Brown 24 [C.I. 77310, (CAS: 68186-90-3)], Pigment Brown 26 [C.I. 71129, (CAS: 81-33-4)], Pigment Brown 27 [C.I. 73410, (CAS: 3989-75-1)], Pigment Brown 28 [C.I. 69015, (CAS: 131-92-0)], Pigment Brown 33 [C.I. 77503, (CAS: 68186-88-9)], Pigment Brown 34 [C.I. 77497, (CAS: 68187-10-0)], Pigment Brown 35 [C.I. 77501, (CAS: 68187-09-7)], Pigment Brown 37 [C.I. 77890, (CAS: 70248-09-8)], Pigment Brown 38 [CI. 561660, (CAS: 126338-72-5)], Pigment Brown 39 [C.I. 77312, (CAS: 71750-83-9)], Pigment Brown 6 [C.I. 77491, 77492 and 77499, (CAS: 52357-70-7)], Pigment Brown 9 [C.I. 77430, (CAS: 8014-85-5)], Pigment Green 10 [CI. 12775, (CAS: 61725-51-7)], Pigment Green 12 [CI. 10020, (CAS: 84682-41-7)], Pigment Green 15 [C.I. 77600, (CAS: 12224-92-9)], Pigment Green 17 [C.I. 77288, (CAS: 1308-38-9)], Pigment Green 18 [C.I. 77289, (CAS: 12001-99-9)], Pigment Green 19 [C.I. 77335, (CAS: 8011-87-8)], Pigment Green 20 [C.I. 77408, (CAS: 8007-61-2)], Pigment Green 21 [C.I. 77410, (CAS: 12002-03-8)], Pigment Green 22 [C.I. 77412, (CAS: 1345-20-6)], Pigment Green 23 [C.I. 77009, (CAS: 1344-98-5)], Pigment Green 24 [C.I. 77013, (CAS: 1345-00-2)], Pigment Green 26 [C.I. 77344, (CAS: 68187-49-5)], Pigment Green 27 [C.I. 77520, (CAS: 15418-51-6)], Pigment Green 36 [C.I. 74265, (CAS: 14302-13-7)], Pigment Green 37 [C.I. 74255, (CAS: 1330-37-6)], Pigment Green 38 [C.I. 74265, (CAS: 14302-13-7)], Pigment Green 42 [CI. 74260, (CAS: 1328-53-6)], Pigment Green 47 [C.I. 59825, (CAS: 128-58-5)], Pigment Green 50 [C.I. 77377, (CAS: 68186-85-6)], Pigment Green 51 [C.I. 77300, (CAS: 68553-01-5)], Pigment Green 54 [C.I. 59830, (CAS: 25704-81-8)], Pigment Green 58 [C.I. 742655, (CAS: 1143572-73-9)], Pigment Green 8 [C.I. 10006, (CAS: 16143-80-9)], Pigment Green 9 [CI. 49415, (CAS: 1326-13-2)], Pigment Orange 1 [C.I. 11725, (CAS: 6371-96-6)], Pigment Orange 13 [C.I. 21110, (CAS: 3520-72-7)], Pigment Orange 14 [C.I. 21165, (CAS: 6837-37-2)], Pigment Orange 15 [C.I. 21130, (CAS: 6358-88-9)], Pigment Orange 16 [CI. 21160, (CAS: 6505-28-8)], Pigment Orange 17 [C.I. 15510, (CAS: 15782-04-4)], Pigment Orange 17 [C.I. 15510, (CAS: 15876-51-4)], Pigment Orange 18 [CI. 15970, (CAS: 1325-14-0)], Pigment Orange 19 [C.I. 15990, (CAS: 5858-88-8)], Pigment Orange 20 [C.I. 77202, (CAS: 12656-57-4)], Pigment Orange 21 [C.I. 77601, (CAS: 1344-38-3)], Pigment Orange 22 [C.I. 12470, (CAS: 6358-48-1)], Pigment Orange 23 [CI. 77201, (CAS: 1345-09-1)], Pigment Orange 24 [C.I. 12305, (CAS: 6410-27-1)], Pigment Orange 3 [C.I. 12105, (CAS: 6410-15-7)], Pigment Orange 31 [CI. 20050, (CAS: 5280-74-0)], Pigment Orange 34 [C.I. 21115, (CAS: 15793-73-4)], Pigment Orange 39 [C.I. 45370, (CAS: 15876-57-0)], Pigment Orange 4 [C.I. 12459, (CAS: 21889-27-0)], Pigment Orange 40 [C.I. 59700, (CAS: 128-70-1)], Pigment Orange 43 [C.I. 71105, (CAS: 4424-06-0)], Pigment Orange 44 [C.I. 21162, (CAS: 17453-73-5)], Pigment Orange 45 [C.I. 77601, (CAS: 59519-55-0)], Pigment Orange 46 [C.I. 15602, (CAS: 63467-26-5)], Pigment Orange 5 [C.I. 12075, (CAS: 3468-63-1)], Pigment Orange 6 [C.I. 12730, (CAS: 6407-77-8)], Pigment Orange 61 [C.I. 11265, (CAS: 40716-47-0)], Pigment Orange 64 [C.I. 12760, (CAS: 72102-84-2)], Pigment Orange 65 [CI. 48053, (CAS: 20437-10-9)], Pigment Orange 66 [C.I. 48210, (CAS: 68808-69-5)], Pigment Orange 67 [C.I. 12915, (CAS: 74336-59-7)], Pigment Orange 68 [C.I. 486150, (CAS: 42844-93-9)], Pigment Orange 69 [CI. 56292, (CAS: 85959-60-0)], Pigment Orange 7 [C.I. 15530, (CAS: 5850-81-7)], Pigment Orange 71 [C.I. 561200, (CAS: 84632-50-8)], Pigment Orange 72 [C.I. 211095, (CAS: 384329-80-0)], Pigment Orange 73 [C.I. 561170, (CAS: 84632-59-7)], Pigment Orange 75 [CI. 772830, (CAS: 12014-93-6)], Pigment Orange 77 [C.I. 59105, (CAS: 1324-11-4)], Pigment Red 10 [C.I. 12440, (CAS: 6410-35-1)], Pigment Red 100 [C.I. 13058, (CAS: 6371-55-7)], Pigment Red 101 [C.I. 77491, (CAS: 1309-37-1)], Pigment Red 101 [C.I. 77015, (CAS: 529484-30-8)], Pigment Red 103 [C.I. 77601, (CAS: 59519-56-1)], Pigment Red 104 [CI. 77605, (CAS: 12656-85-8)], Pigment Red 105 [C.I. 77578, (CAS: 1314-41-6)], Pigment Red 106 [C.I. 77766, (CAS: 1344-48-5)], Pigment Red 107 [C.I. 77060, (CAS: 1345-04-6)], Pigment Red 108 [CI. 77202, (CAS: 58339-34-7)], Pigment Red 109 [C.I. 77482, (CAS: 1345-24-0)], Pigment Red 11 [C.I. 12430, (CAS: 6535-48-4)], Pigment Red 112 [C.I. 12370, (CAS: 6535-46-2)], Pigment Red 113 [CI. 77201, (CAS: 1345-09-1)], Pigment Red 114 [CI. 12351, (CAS: 6358-47-0)], Pigment Red 115 [CI. 15851, (CAS: 6358-40-3)], Pigment Red 117 [C.I. 15603, (CAS: 10142-77-5)], Pigment Red 119 [C.I. 12469, (CAS: 72066-77-4)], Pigment Red 12 [C.I. 12385, (CAS: 6410-32-8)], Pigment Red 121 [C.I. 77302, (CAS: 12125-42-7)], Pigment Red 122 [C.I. 73915, (CAS: 980-26-7)], Pigment Red 13 [C.I. 12395, (CAS: 6535-47-3)], Pigment Red 133 [CI. 15920, (CAS: 5280-67-1)], Pigment Red 14 [C.I. 12380, (CAS: 6471-50-7)], Pigment Red 141 [CI. 20044, (CAS: 3864-06-0)], Pigment Red 144 [C.I. 20735, (CAS: 5280-78-4)], Pigment Red 146 [CI. 12485, (CAS: 5280-68-2)], Pigment Red 147 [C.I. 12433, (CAS: 68227-78-1)], Pigment Red 148 [C.I. 12369, (CAS: 94276-08-1)], Pigment Red 149 [C.I. 71137, (CAS: 4948-15-6)], Pigment Red 15 [C.I. 12465, (CAS: 6410-39-5)], Pigment Red 150 [C.I. 12290, (CAS: 56396-10-2)], Pigment Red 151 [C.I. 15892, (CAS: 61013-97-6)], Pigment Red 157 [C.I. 12355, (CAS: 6471-49-4)], Pigment Red 16 [CI. 12500, (CAS: 6407-71-2)], Pigment Red 162 [C.I. 12431, (CAS: 6358-59-4)], Pigment Red 163 [CI. 12455, (CAS: 6410-37-3)], Pigment Red 164 [C.I. 212855, (CAS: 72659-69-9)], Pigment Red 166 [CI. 20730, (CAS: 3905-19-9)], Pigment Red 168 [C.I. 59300, (CAS: 4378-61-4)], Pigment Red 169 [C.I. 45160, (CAS: 12237-63-7)], Pigment Red 17 [CI. 12390, (CAS: 6655-84-1)], Pigment Red 170 [CI. 12475, (CAS: 2786-76-7)], Pigment Red 170 [CI. 12474, (CAS: 36968-27-1)], Pigment Red 171 [C.I. 12512, (CAS: 6985-95-1)], Pigment Red 172 [CI. 45430, (CAS: 12227-78-0)], Pigment Red 173 [C.I. 45170, (CAS: 12227-77-9)], Pigment Red 174 [C.I. 45410, (CAS: 15876-58-1)], Pigment Red 175 [CI. 12513, (CAS: 6985-92-8)], Pigment Red 177 [C.I. 65300, (CAS: 4051-63-2)], Pigment Red 179 [C.I. 71130, (CAS: 5521-31-3)], Pigment Red 18 [CI. 12350, (CAS: 3564-22-5)], Pigment Red 181 [CI. 73360, (CAS: 2379-74-0)], Pigment Red 184 [C.I. 12487, (CAS: 99402-80-9)], Pigment Red 185 [C.I. 12516, (CAS: 51920-12-8)], Pigment Red 187 [C.I. 12486, (CAS: 59487-23-9)], Pigment Red 188 [C.I. 12467, (CAS: 61847-48-1)], Pigment Red 189 [C.I. 71135, (CAS: 2379-77-3)], Pigment Red 19 [C.I. 12400, (CAS: 6410-33-9)], Pigment Red 190 [CI. 71140, (CAS: 6424-77-7)], Pigment Red 192 [C.I. 739155, (CAS: 61968-81-8)], Pigment Red 193 [C.I. 16185, (CAS: 12227-62-2)], Pigment Red 195 [C.I. 70320, (CAS: 4203-77-4)], Pigment Red 196 [C.I. 67000, (CAS: 2379-79-5)], Pigment Red 198 [C.I. 73390, (CAS: 6371-31-9)], Pigment Red 2 [C.I. 12310, (CAS: 6041-94-7)], Pigment Red 200 [C.I. 15867, (CAS: 58067-05-3)], Pigment Red 200 [C.I. 15867, (CAS: 32041-58-0)], Pigment Red 202 [C.I. 73907, (CAS: 3089-17-6)], Pigment Red 208 [CI. 12514, (CAS: 31778-10-6)], Pigment Red 21 [C.I. 12300, (CAS: 6410-26-0)], Pigment Red 210 [C.I. 12477, (CAS: 61932-63-6)], Pigment Red 211 [C.I. 15910, (CAS: 85702-54-1)], Pigment Red 212 [CI. 12360, (CAS: 6448-96-0)], Pigment Red 214 [CI. 200660, (CAS: 40618-31-3)], Pigment Red 216 [CI. 59710, (CAS: 1324-33-0)], Pigment Red 22 [C.I. 12315, (CAS: 6448-95-9)], Pigment Red 220 [C.I. 20055, (CAS: 68259-05-2)], Pigment Red 221 [C.I. 20065, (CAS: 71566-54-6)], Pigment Red 222 [CI. 123665, (CAS: 20981-12-8)], Pigment Red 224 [C.I. 71127, (CAS: 128-69-8)], Pigment Red 226 [CI. 597200, (CAS: 72828-01-4)], Pigment Red 229 [CI. 77006, (CAS: 85536-78-3)], Pigment Red 230 [C.I. 77003, (CAS: 68187-27-9)], Pigment Red 231 [C.I. 77005, (CAS: 68186-99-2)], Pigment Red 232 [C.I. 77996, (CAS: 68412-79-3)], Pigment Red 233 [C.I. 77301, (CAS: 68187-12-2)], Pigment Red 235 [C.I. 77290, (CAS: 68201-65-0)], Pigment Red 236 [CI. 77863, (CAS: 68187-53-1)], Pigment Red 242 [C.I. 20067, (CAS: 52238-92-3)], Pigment Red 243 [CI. 15910, (CAS: 50326-33-5)], Pigment Red 243 [CI. 15910, (CAS: 431991-58-1)], Pigment Red 247 [CI. 15915, (CAS: 43035-18-3)], Pigment Red 248 [C.I. 200552, (CAS: 80648-58-4)], Pigment Red 251 [CI. 12925, (CAS: 74336-60-0)], Pigment Red 253 [C.I. 12375, (CAS: 85776-13-2)], Pigment Red 254 [C.I. 56110, (CAS: 84632-65-5)], Pigment Red 255 [C.I. 561050, (CAS: 54660-00-3)], Pigment Red 256 [C.I. 124635, (CAS: 79102-65-1)], Pigment Red 257 [C.I. 562700, (CAS: 70833-37-3)], Pigment Red 258 [C.I. 12318, (CAS: 57301-22-1)], Pigment Red 259 [CI. 77007, (CAS: 113956-14-2)], Pigment Red 260 [C.I. 56295, (CAS: 71552-60-8)], Pigment Red 261 [CI. 12468, (CAS: 16195-23-6)], Pigment Red 264 [CI. 561300, (CAS: 88949-33-1)], Pigment Red 265 [CI. 772830, (CAS: 12014-93-6)], Pigment Red 267 [C.I. 12396, (CAS: 68016-06-8)], Pigment Red 268 [CI. 12316, (CAS: 16403-84-2)], Pigment Red 269 [C.I. 12466, (CAS: 67990-05-0)], Pigment Red 271 [C.I. 487100, (CAS: 85958-80-1)], Pigment Red 273 [CI. 16035, (CAS: 68583-95-9)], Pigment Red 274 [C.I. 16255, (CAS: 12227-64-4)], Pigment Red 3 [C.I. 12120, (CAS: 2425-85-6)], Pigment Red 30 [C.I. 12330, (CAS: 6471-48-3)], Pigment Red 32 [CI. 12320, (CAS: 6410-29-3)], Pigment Red 37 [C.I. 21205, (CAS: 6883-91-6)], Pigment Red 38 [C.I. 21120, (CAS: 6358-87-8)], Pigment Red 39 [C.I. 21080, (CAS: 6492-54-2)], Pigment Red 4 [CI. 12085, (CAS: 2814-77-9)], Pigment Red 40 [C.I. 12170, (CAS: 2653-64-7)], Pigment Red 41 [C.I. 21200, (CAS: 6505-29-9)], Pigment Red 42 [C.I. 21210, (CAS: 6358-90-3)], Pigment Red 48 [C.I. 15865, (CAS: 3564-21-4)], Pigment Red 48 [C.I. 15865, (CAS: 1325-12-8)], Pigment Red 48 [C.I. 15865, (CAS: 7585-41-3)], Pigment Red 48 [C.I. 15865, (CAS: 7023-61-2)], Pigment Red 48 [C.I. 15865, (CAS: 15782-05-5)], Pigment Red 48 [C.I. 15865, (CAS: 5280-66-0)], Pigment Red 48 [C.I. 15865, (CAS: 71832-83-2)], Pigment Red 48 [C.I. 15865, (CAS: 68966-97-2)], Pigment Red 49 [C.I. 15630, (CAS: 1248-18-6)], Pigment Red 49 [C.I. 15630, (CAS: 1325-06-0)], Pigment Red 49 [C.I. 15630, (CAS: 1103-38-4)], Pigment Red 49 [C.I. 15630, (CAS: 1103-39-5)], Pigment Red 49 [C.I. 15630, (CAS: 6371-67-1)], Pigment Red 5 [C.I. 12490, (CAS: 6410-41-9)], Pigment Red 50 [C.I. 15500, (CAS: 5850-76-0)], Pigment Red 50 [C.I. 15500, (CAS: 6372-81-2)], Pigment Red 51 [C.I. 15580, (CAS: 5850-87-3)], Pigment Red 52 [C.I. 15860, (CAS: 5858-82-2)], Pigment Red 52 [C.I. 15860, (CAS: 1325-11-7)], Pigment Red 52 [C.I. 15860, (CAS: 17852-99-2)], Pigment Red 52 [C.I. 15860, (CAS: 17814-20-9)], Pigment Red 52 [C.I. 15860, (CAS: 12238-31-2)], Pigment Red 53 [C.I. 15585, (CAS: 2092-56-0)], Pigment Red 53 [C.I. 15585, (CAS: 1325-04-8)], Pigment Red 53 [C.I. 15585, (CAS: 67990-35-6)], Pigment Red 53 [C.I. 15585, (CAS: 73263-40-8)], Pigment Red 54 [C.I. 14830, (CAS: 6373-10-0)], Pigment Red 55 [C.I. 15820, (CAS: 141052-43-9)], Pigment Red 57 [C.I. 15850, (CAS: 5858-81-1)], Pigment Red 57 [C.I. 15850, (CAS: 17852-98-1)], Pigment Red 57 [CI. 15850, (CAS: 55491-44-6)], Pigment Red 58 [C.I. 15825, (CAS: 1325-09-3)], Pigment Red 58 [C.I. 15825, (CAS: 7538-59-2)], Pigment Red 58 [C.I. 15825, (CAS: 15782-03-3)], Pigment Red 58 [C.I. 15825, (CAS: 76613-71-3)], Pigment Red 58 [C.I. 15825, (CAS: 64552-28-9)], Pigment Red 6 [C.I. 12090, (CAS: 6410-13-5)], Pigment Red 60 [C.I. 16105, (CAS: 15782-06-6)], Pigment Red 60 [C.I. 16105, (CAS: 1325-16-2)], Pigment Red 61 [C.I. 24830, (CAS: 1325-29-7)], Pigment Red 62 [C.I. 23295, (CAS: 109823-18-9)], Pigment Red 63 [C.I. 15880, (CAS: 21416-46-6)], Pigment Red 63 [C.I. 15880, (CAS: 6417-83-0)], Pigment Red 63 [C.I. 15880, (CAS: 15792-20-8)], Pigment Red 63 [C.I. 15880, (CAS: 35355-77-2)], Pigment Red 64 [C.I. 15800, (CAS: 16508-79-5)], Pigment Red 64 [C.I. 15800, (CAS: 6371-76-2)], Pigment Red 65 [C.I. 18020, (CAS: 1325-21-9)], Pigment Red 66 [C.I. 18000, (CAS: 1325-19-5)], Pigment Red 67 [C.I. 18025, (CAS: 1325-22-0)], Pigment Red 68 [C.I. 15525, (CAS: 5850-80-6)], Pigment Red 69 [C.I. 15595, (CAS: 5850-90-8)], Pigment Red 7 [CI. 12420, (CAS: 6471-51-8)], Pigment Red 70 [C.I. 15590, (CAS: 5850-89-5)], Pigment Red 77 [C.I. 15826, (CAS: 6358-39-0)], Pigment Red 8 [C.I. 12335, (CAS: 6410-30-6)], Pigment Red 83 [C.I. 58000, (CAS: 104074-25-1)], Pigment Red 84 [C.I. 58210, (CAS: 1328-07-0)], Pigment Red 85 [C.I. 63350, (CAS: 6370-96-3)], Pigment Red 86 [C.I. 73375, (CAS: 6371-26-2)], Pigment Red 89 [C.I. 60745, (CAS: 6409-74-1)], Pigment Red 9 [C.I. 12460, (CAS: 6410-38-4)], Pigment Red 90 [C.I. 45380, (CAS: 15876-39-8)], Pigment Red 93 [C.I. 12152, (CAS: 6548-36-3)], Pigment Red 95 [C.I. 15897, (CAS: 72639-39-5)], Pigment Red 99 [CI. 15570, (CAS: 5850-85-1)], Pigment Violet 10 [C.I. 42535, (CAS: 1325-82-2)], Pigment Violet 12 [C.I. 58050, (CAS: 1328-03-6)], Pigment Violet 13 [C.I. 125085, (CAS: 83399-83-1)], Pigment Violet 14 [C.I. 77360, (CAS: 10101-56-1)], Pigment Violet 15 [C.I. 77007, (CAS: 12769-96-9)], Pigment Violet 16 [C.I. 77742, (CAS: 10101-66-3)], Pigment Violet 19 [C.I. 46500, (CAS: 1047-16-1)], Pigment Violet 20 [CI. 58225, (CAS: 6486-92-6)], Pigment Violet 23 [C.I. 51319, (CAS: 215247-95-3)], Pigment Violet 25 [C.I. 12321, (CAS: 6358-46-9)], Pigment Violet 27 [C.I. 42535, (CAS: 12237-62-6)], Pigment Violet 29 [C.I. 71129, (CAS: 81-33-4)], Pigment Violet 3 [CI. 42535, (CAS: 68647-35-8)], Pigment Violet 3 [C.I. 42535, (CAS: 68308-41-8)], Pigment Violet 3 [C.I. 42535, (CAS: 67989-22-4)], Pigment Violet 31 [C.I. 60010, (CAS: 1324-55-6)], Pigment Violet 33 [C.I. 60005, (CAS: 1324-17-0)], Pigment Violet 36 [C.I. 73385, (CAS: 5462-29-3)], Pigment Violet 37 [C.I. 51345, (CAS: 17741-63-8)], Pigment Violet 38 [C.I. 73395, (CAS: 2379-75-1)], Pigment Violet 47 [C.I. 77363, (CAS: 68610-13-9)], Pigment Violet 48 [C.I. 77352, (CAS: 68608-93-5)], Pigment Violet 49 [C.I. 77362, (CAS: 16827-96-6)], Pigment Violet 5 [C.I. 58055, (CAS: 1328-04-7)], Pigment Violet 6 [C.I. 58060, (CAS: 6483-85-8)], Pigment Violet 6 [C.I. 58060, (CAS: 1328-05-8)], Pigment Violet 7 [C.I. 58065, (CAS: 1328-06-9)], Pigment Violet 8 [C.I. 18005, (CAS: 1325-20-8)], Pigment Yellow 1 [C.I. 11680, (CAS: 2512-29-0)], Pigment Yellow 10 [CI. 12710, (CAS: 6407-75-6)], Pigment Yellow 100 [C.I. 19140, (CAS: 12225-21-7)], Pigment Yellow 104 [C.I. 15985, (CAS: 15790-07-5)], Pigment Yellow 105 [C.I. 11743, (CAS: 12236-75-8)], Pigment Yellow 109 [C.I. 56284, (CAS: 5045-40-9)], Pigment Yellow 11 [C.I. 10325, (CAS: 2955-16-0)], Pigment Yellow 110 [C.I. 56280, (CAS: 5590-18-1)], Pigment Yellow 111 [C.I. 11745, (CAS: 15993-42-7)], Pigment Yellow 112 [C.I. 70600, (CAS: 475-71-8)], Pigment Yellow 114 [CI. 21092, (CAS: 68610-87-7)], Pigment Yellow 115 [CI. 47005, (CAS: 68814-04-0)], Pigment Yellow 116 [CI. 11790, (CAS: 61968-84-1)], Pigment Yellow 117 [CI. 48043, (CAS: 21405-81-2)], Pigment Yellow 118 [C.I. 77894, (CAS: 61512-65-0)], Pigment Yellow 119 [C.I. 77496, (CAS: 68187-51-9)], Pigment Yellow 12 [C.I. 21090, (CAS: 6358-85-6)], Pigment Yellow 123 [C.I. 65049, (CAS: 4028-94-8)], Pigment Yellow 124 [C.I. 21107, (CAS: 67828-22-2)], Pigment Yellow 126 [C.I. 21101, (CAS: 90268-23-8)], Pigment Yellow 127 [C.I. 21102, (CAS: 68610-86-6)], Pigment Yellow 128 [CI. 20037, (CAS: 79953-85-8)], Pigment Yellow 129 [C.I. 48042, (CAS: 15680-42-9)], Pigment Yellow 13 [C.I. 21100, (CAS: 5102-83-0)], Pigment Yellow 130 [C.I. 117699, (CAS: 23739-66-4)], Pigment Yellow 133 [C.I. 139395, (CAS: 85702-53-0)], Pigment Yellow 134 [CI. 21111, (CAS: 31775-20-9)], Pigment Yellow 138 [C.I. 56300, (CAS: 30125-47-4)], Pigment Yellow 139 [C.I. 56298, (CAS: 36888-99-0)], Pigment Yellow 14 [C.I. 21095, (CAS: 5468-75-7)], Pigment Yellow 147 [C.I. 60645, (CAS: 4118-16-5)], Pigment Yellow 148 [CI. 50600, (CAS: 20572-37-6)], Pigment Yellow 15 [C.I. 21220, (CAS: 6528-35-4)], Pigment Yellow 150 [CI. 12764, (CAS: 872613-79-1)], Pigment Yellow 153 [CI. 48545, (CAS: 29204-84-0)], Pigment Yellow 155 [CI. 200310, (CAS: 68516-73-4)], Pigment Yellow 157 [C.I. 77900, (CAS: 68610-24-2)], Pigment Yellow 158 [C.I. 77862, (CAS: 68186-93-6)], Pigment Yellow 159 [C.I. 77997, (CAS: 68187-15-5)], Pigment Yellow 16 [C.I. 20040, (CAS: 5979-28-2)], Pigment Yellow 160 [CI. 77991, (CAS: 68187-01-9)], Pigment Yellow 161 [C.I. 77895, (CAS: 68611-43-8)], Pigment Yellow 162 [CI. 77896, (CAS: 68611-42-7)], Pigment Yellow 163 [C.I. 77897, (CAS: 68186-92-5)], Pigment Yellow 164 [C.I. 77899, (CAS: 68412-38-4)], Pigment Yellow 167 [C.I. 11737, (CAS: 38489-24-6)], Pigment Yellow 168 [CI. 13960, (CAS: 71832-85-4)], Pigment Yellow 169 [C.I. 13955, (CAS: 73385-03-2)], Pigment Yellow 17 [C.I. 21105, (CAS: 4531-49-1)], Pigment Yellow 173 [C.I. 561600, (CAS: 51016-63-8)], Pigment Yellow 174 [C.I. 21098, (CAS: 78952-72-4)], Pigment Yellow 176 [C.I. 21103, (CAS: 90268-24-9)], Pigment Yellow 177 [C.I. 48120, (CAS: 60109-88-8)], Pigment Yellow 179 [C.I. 48125, (CAS: 63287-28-5)], Pigment Yellow 180 [C.I. 21290, (CAS: 77804-81-0)], Pigment Yellow 181 [C.I. 11777, (CAS: 74441-05-7)], Pigment Yellow 182 [C.I. 128300, (CAS: 67906-31-4)], Pigment Yellow 183 [C.I. 18792, (CAS: 65212-77-3)], Pigment Yellow 184 [C.I. 771740, (CAS: 14059-33-7)], Pigment Yellow 185 [CI. 56290, (CAS: 76199-85-4)], Pigment Yellow 188 [C.I. 21094, (CAS: 23792-68-9)], Pigment Yellow 190 [C.I. 189785, (CAS: 94612-75-6)], Pigment Yellow 191 [C.I. 18795, (CAS: 129423-54-7)], Pigment Yellow 191 [CI. 18795, (CAS: 154946-66-4)], Pigment Yellow 192 [C.I. 507300, (CAS: 56279-27-7)], Pigment Yellow 193 [CI. 65412, (CAS: 70321-14-1)], Pigment Yellow 194 [C.I. 11785, (CAS: 82199-12-0)], Pigment Yellow 199 [C.I. 653200, (CAS: 136897-58-0)], Pigment Yellow 2 [C.I. 11730, (CAS: 6486-26-6)], Pigment Yellow 202 [C.I. 65410, (CAS: 3627-47-2)], Pigment Yellow 203 [CI. 117390, (CAS: 150959-17-4)], Pigment Yellow 213 [C.I. 117875, (CAS: 220198-21-0)], Pigment Yellow 218 [C.I. 561805, (CAS: 910868-14-3)], Pigment Yellow 220 [C.I. 561806, (CAS: 17352-39-5)], Pigment Yellow 227 [CI. 777895, (CAS: 1374645-21-2)], Pigment Yellow 3 [C.I. 11710, (CAS: 6486-23-3)], Pigment Yellow 30 [CI. 77592, (CAS: 1345-30-8)], Pigment Yellow 31 [CI. 77103, (CAS: 10294-40-3)], Pigment Yellow 33 [C.I. 77223, (CAS: 8012-75-7)], Pigment Yellow 34 [C.I. 77603, (CAS: 1344-37-2)], Pigment Yellow 35 [C.I. 77205, (CAS: 90604-89-0)], Pigment Yellow 36 [C.I. 77956, (CAS: 49663-84-5)], Pigment Yellow 37 [C.I. 77199, (CAS: 90604-90-3)], Pigment Yellow 38 [C.I. 77878, (CAS: 1315-01-1)], Pigment Yellow 39 [C.I. 77086, (CAS: 1303-33-9)], Pigment Yellow 4 [C.I. 11665, (CAS: 1657-16-5)], Pigment Yellow 41 [C.I. 77588, (CAS: 8012-00-8)], Pigment Yellow 42 [C.I. 77492, (CAS: 51274-00-1)], Pigment Yellow 43 [C.I. 77492, (CAS: 64294-91-3)], Pigment Yellow 44 [CI. 77188, (CAS: 1345-08-0)], Pigment Yellow 45 [CI. 77505, (CAS: 1328-64-9)], Pigment Yellow 46 [C.I. 77577, (CAS: 1317-36-8)], Pigment Yellow 48 [C.I. 77610, (CAS: 592-05-2)], Pigment Yellow 5 [C.I. 11660, (CAS: 4106-67-6)], Pigment Yellow 53 [C.I. 77788, (CAS: 8007-18-9)], Pigment Yellow 55 [C.I. 21096, (CAS: 6358-37-8)], Pigment Yellow 6 [C.I. 11670, (CAS: 4106-76-7)], Pigment Yellow 60 [C.I. 12705, (CAS: 6407-74-5)], Pigment Yellow 61 [C.I. 13880, (CAS: 5280-69-3)], Pigment Yellow 62 [C.I. 13940, (CAS: 12286-66-7)], Pigment Yellow 62 [C.I. 13940, (CAS: 5280-70-6)], Pigment Yellow 65 [C.I. 11740, (CAS: 6528-34-3)], Pigment Yellow 7 [C.I. 12780, (CAS: 6407-81-4)], Pigment Yellow 73 [CI. 11738, (CAS: 13515-40-7)], Pigment Yellow 74 [C.I. 11741, (CAS: 6358-31-2)], Pigment Yellow 75 [C.I. 11770, (CAS: 52320-66-8)], Pigment Yellow 77 [C.I. 20045, (CAS: 5905-17-9)], Pigment Yellow 81 [C.I. 21127, (CAS: 22094-93-5)], Pigment Yellow 83 [C.I. 21108, (CAS: 5567-15-7)], Pigment Yellow 83 [C.I. 21107, (CAS: 15110-84-6)], Pigment Yellow 9 [C.I. 11720, (CAS: 6486-24-4)], Pigment Yellow 93 [C.I. 20710, (CAS: 5580-57-4)], Pigment Yellow 94 [C.I. 20038, (CAS: 5580-58-5)], Pigment Yellow 95 [C.I. 20034, (CAS: 5280-80-8)], Pigment Yellow 98 [C.I. 11727, (CAS: 32432-45-4)], Prussian blue [C.I. 77510, (CAS: 12240-15-2)], Pigment Blue 1 [(CAS: 1325-87-7)], Pigment Blue 1 [(CAS: 69980-72-9)], Pigment Blue 1 [(CAS: 68409-66-5)], Pigment Blue 10 [(CAS: 84057-86-3)], Pigment Blue 12 [(CAS: 1325-77-5)], Pigment Blue 14 [(CAS: 1325-88-8)], Pigment Blue 2 [(CAS: 1325-94-6)], Pigment Blue 3 [(CAS: 1325-79-7)], Pigment Blue 9 [(CAS: 1325-74-2)], Pigment Green 1 [(CAS: 1325-75-3)], Pigment Green 3 [(CAS: 68845-37-4)], Pigment Green 4 [(CAS: 61725-50-6)], Pigment Red 80 [(CAS: 12224-98-5)], Pigment Red 81 [(CAS: 80083-40-5)], Pigment Red 81 [(CAS: 75627-12-2)], Pigment Red 81 [(CAS: 68310-07-6)], Pigment Red 81 [(CAS: 85959-61-1)], Pigment Red 81 [(CAS: 63022-06-0)], Pigment Red 81 [(CAS: 63022-07-1)], Pigment Violet 1 [(CAS: 1326-03-0)], Pigment Violet 2 [(CAS: 1326-04-1)], Pigment Violet 2 [(CAS: 103443-41-0)], Pigment Violet 4 [(CAS: 1325-80-0)], Pigment Black 1 [(CAS: 73104-73-1)], Pigment Black 1 [(CAS: 9064-44-2)], Pigment Black 11 [(CAS: 120899-48-1)], Pigment Black 11 [(CAS: 128666-38-6)], Pigment Black 11 [(CAS: 128666-37-5)], Pigment Black 11 [(CAS: 128666-36-4)], Pigment Black 11 [(CAS: 147858-25-1)], Pigment Black 16 [(CAS: 7440-66-6)], Pigment Black 19 [(CAS: 874954-47-9)], Pigment Black 2 [(CAS: 12236-57-6)], Pigment Black 20 [(CAS: 12216-93-2)], Pigment Black 21 [(CAS: 12216-94-3)], Pigment Black 22 [(CAS: 55353-02-1)], Pigment Black 3 [(CAS: 945563-42-8)], Pigment Black 35 [(CAS: 945563-51-9)], Pigment Black 5 [(CAS: 945563-45-1)], Pigment Blue 1 [(CAS: 68647-33-6)], Pigment Blue 10 [(CAS: 308086-15-9)], Pigment Blue 11 [(CAS: 71798-70-4)], Pigment Blue 13 [(CAS: 945558-73-6)], Pigment Blue 15-Pigment Green 7 mixt. [(CAS: 1026025-11-5)], Pigment Blue 15-Pigment Red 122-Pigment Yellow 74 mixt. [(CAS: 1357447-02-9)], Pigment Blue 151 [(CAS: 685529-31-1)], Pigment Blue 16 [(CAS: 424827-05-4)], Pigment Blue 17 [(CAS: 153640-87-0)], Pigment Blue 17 [(CAS: 71799-04-7)], Pigment Blue 19 [(CAS: 58569-23-6)], Pigment Blue 2 [(CAS: 1126074-38-1)], Pigment Blue 20 [(CAS: 945558-74-7)], Pigment Blue 209 [(CAS: 215590-82-2)], Pigment Blue 23 [(CAS: 57486-30-3)], Pigment Blue 24 [(CAS: 1042940-03-3)], Pigment Blue 28 [(CAS: 151732-17-1)], Pigment Blue 29 [(CAS: 151732-19-3)], Pigment Blue 31 [(CAS: 945558-75-8)], Pigment Blue 4 [(CAS: 945558-70-3)], Pigment Blue 5 [(CAS: 945558-72-5)], Pigment Blue 52 [(CAS: 945558-90-7)], Pigment Blue 53 [(CAS: 945558-91-8)], Pigment Blue 53 [(CAS: 190454-42-3)], Pigment Blue 56 [(CAS: 64427-27-6)], Pigment Blue 58 [(CAS: 12236-58-7)], Pigment Blue 59 [(CAS: 12236-59-8)], Pigment Blue 6 [(CAS: 371759-37-4)], Pigment Blue 61 [(CAS: 1126075-97-5)], Pigment Blue 63 [(CAS: 815586-00-6)], Pigment Blue 67 [(CAS: 945558-93-0)], Pigment Blue 68 [(CAS: 129406-28-6)], Pigment Blue 69 [(CAS: 945558-94-1)], Pigment Blue 7 [(CAS: 71838-91-0)], Pigment Blue 7 [(CAS: 120177-75-5)], Pigment Blue 70 [(CAS: 72827-99-7)], Pigment Blue 77 [(CAS: 945558-95-2)], Pigment Blue 8 [(CAS: 12224-90-7)], Pigment Blue 80 [(CAS: 391663-82-4)], Pigment Blue 81 [(CAS: 945558-98-5)], Pigment Blue 83 [(CAS: 1126076-49-0)], Pigment Blue 84 [(CAS: 2095508-48-6)], Pigment Brown 126 [(CAS: 128664-60-8)], Pigment Brown 29 [(CAS: 109414-04-2)], Pigment Brown 3 [(CAS: 1325-24-2)], Pigment Brown 30 [(CAS: 135668-57-4)], Pigment Brown 31 [(CAS: 126338-71-4)], Pigment Brown 32 [(CAS: 72828-00-3)], Pigment Brown 36 [(CAS: 945563-08-6)], Pigment Brown 4 [(CAS: 109944-91-4)], Pigment Brown 40 [(CAS: 945563-13-3)], Pigment Brown 41 [(CAS: 211502-16-8)], Pigment Brown 42 [(CAS: 211502-17-9)], Pigment Brown 43 [(CAS: 75864-23-2)], Pigment Brown 44 [(CAS: 945563-18-8)], Pigment Brown 45 [(CAS: 945563-37-1)], Pigment Brown 46 [(CAS: 945563-38-2)], Pigment Brown 47 [(CAS: 945563-39-3)], Pigment Brown 48 [(CAS: 2170864-80-7)], Pigment Brown 5 [(CAS: 16521-34-9)], Pigment Brown 6 [(CAS: 1275574-14-5)], Pigment Green 1 [(CAS: 68814-00-6)], Pigment Green 1 [(CAS: 68123-12-6)], Pigment Green 13 [(CAS: 148092-61-9)], Pigment Green 14 [(CAS: 114013-40-0)], Pigment Green 16 [(CAS: 65505-26-2)], Pigment Green 2 [(CAS: 12213-69-3)], Pigment Green 2 [(CAS: 76963-33-2)], Pigment Green 25 [(CAS: 945560-75-8)], Pigment Green 45 [(CAS: 945561-39-7)], Pigment Green 46 [(CAS: 945561-40-0)], Pigment Green 48 [(CAS: 945561-55-7)], Pigment Green 49 [(CAS: 945561-56-8)], Pigment Green 52 [(CAS: 945562-08-3)], Pigment Green 55 [(CAS: 945563-02-0)], Pigment Green 56 [(CAS: 945563-05-3)], Pigment Green 59 [(CAS: 2170445-83-5)], Pigment Green 6 [(CAS: 945559-56-8)], Pigment Green 62 [(CAS: 2108056-55-7)], Pigment Green 63 [(CAS: 2108056-56-8)], Pigment Green 7 [(CAS: 68022-83-3)], Pigment Green 77 [(CAS: 12715-62-7)], Pigment Green 7-Pigment Yellow 93 mixt. [(CAS: 1046461-83-9)], Pigment Orange 12 [(CAS: 945426-49-3)], Pigment Orange 20 [(CAS: 957128-28-8)], Pigment Orange 25 [(CAS: 12224-97-4)], Pigment Orange 32 [(CAS: 945426-51-7)], Pigment Orange 36 [(CAS: 12236-62-3)], Pigment Orange 38 [(CAS: 12236-64-5)], Pigment Orange 42 [(CAS: 12768-99-9)], Pigment Orange 43-Pigment Orange 64 mixt. [(CAS: 1046461-84-0)], Pigment Orange 47 [(CAS: 71819-73-3)], Pigment Orange 48 [(CAS: 71819-74-4)], Pigment Orange 49 [(CAS: 71819-75-5)], Pigment Orange 50 [(CAS: 76780-89-7)], Pigment Orange 51 [(CAS: 61512-61-6)], Pigment Orange 52 [(CAS: 61512-62-7)], Pigment Orange 53 [(CAS: 945426-52-8)], Pigment Orange 54 [(CAS: 945426-53-9)], Pigment Orange 55 [(CAS: 304891-88-1)], Pigment Orange 56 [(CAS: 74433-73-1)], Pigment Orange 57 [(CAS: 945426-54-0)], Pigment Orange 58 [(CAS: 945426-55-1)], Pigment Orange 59 [(CAS: 304891-93-8)], Pigment Orange 60 [(CAS: 68399-99-5)], Pigment Orange 62 [(CAS: 52846-56-7)], Pigment Orange 63 [(CAS: 76233-79-9)], Pigment Orange 70 [(CAS: 914936-31-5)], Pigment Orange 74 [(CAS: 516493-26-8)], Pigment Orange 76 [(CAS: 945426-61-9)], Pigment Orange 79 [(CAS: 945426-62-0)], Pigment Orange 8 [(CAS: 945426-48-2)], Pigment Orange 80 [(CAS: 945426-63-1)], Pigment Orange 81 [(CAS: 656223-72-2)], Pigment Orange 82 [(CAS: 2170864-77-2)], Pigment Orange 86 [(CAS: 1883421-38-2)], Pigment Orange 9 [(CAS: 71799-05-8)], Pigment Red 1 [(CAS: 39781-24-3)], Pigment Red 102 [(CAS: 1332-25-8)], Pigment Red 108 [(CAS: 918496-78-3)], Pigment Red 110 [(CAS: 854102-21-9)], Pigment Red 111 [(CAS: 12224-99-6)], Pigment Red 118 [(CAS: 945428-13-7)], Pigment Red 120 [(CAS: 57485-96-8)], Pigment Red 123 [(CAS: 24108-89-2)], Pigment Red 134 [(CAS: 12286-59-8)], Pigment Red 135 [(CAS: 945428-14-8)], Pigment Red 136 [(CAS: 945428-21-7)], Pigment Red 137 [(CAS: 71799-07-0)], Pigment Red 139 [(CAS: 12262-44-1)], Pigment Red 140 [(CAS: 383890-12-8)], Pigment Red 142 [(CAS: 109944-97-0)], Pigment Red 143 [(CAS: 12286-63-4)], Pigment Red 152 [(CAS: 405113-25-9)], Pigment Red 154 [(CAS: 109944-98-1)], Pigment Red 155 [(CAS: 109944-99-2)], Pigment Red 156 [(CAS: 109945-00-8)], Pigment Red 158 [(CAS: 945552-90-9)], Pigment Red 159 [(CAS: 109945-01-9)], Pigment Red 160 [(CAS: 854524-60-0)], Pigment Red 161 [(CAS: 945552-91-0)], Pigment Red 165 [(CAS: 12225-03-5)], Pigment Red 167 [(CAS: 12236-66-7)], Pigment Red 176 [(CAS: 12225-06-8)], Pigment Red 178 [(CAS: 3049-71-6)], Pigment Red 17-Pigment Red 150-Pigment White 18 mixt. [(CAS: 2247196-29-6)], Pigment Red 180 [(CAS: 12769-00-5)], Pigment Red 182 [(CAS: 61036-51-9)], Pigment Red 183 [(CAS: 51920-11-7)], Pigment Red 191 [(CAS: 85068-75-3)], Pigment Red 199 [(CAS: 61901-78-8)], Pigment Red 20 [(CAS: 945426-74-4)], Pigment Red 200 [(CAS: 67801-10-9)], Pigment Red 201 [(CAS: 68258-66-2)], Pigment Red 202-Pigment Violet 19 mixt. [(CAS: 1122063-75-5)], Pigment Red 203 [(CAS: 945553-87-7)], Pigment Red 204 [(CAS: 438231-79-9)], Pigment Red 205 [(CAS: 741692-71-7)], Pigment Red 206 [(CAS: 71819-76-6)], Pigment Red 207 [(CAS: 71819-77-7)], Pigment Red 215 [(CAS: 304892-29-3)], Pigment Red 217 [(CAS: 155421-17-3)], Pigment Red 218 [(CAS: 383891-32-5)], Pigment Red 219 [(CAS: 909006-21-9)], Pigment Red 223 [(CAS: 26789-26-4)], Pigment Red 225 [(CAS: 125270-32-8)], Pigment Red 227 [(CAS: 71872-64-5)], Pigment Red 228 [(CAS: 304898-64-4)], Pigment Red 234 [(CAS: 945554-26-7)], Pigment Red 237 [(CAS: 220424-27-1)], Pigment Red 238 [(CAS: 140114-63-2)], Pigment Red 239 [(CAS: 220424-28-2)], Pigment Red 240 [(CAS: 141489-67-0)], Pigment Red 241 [(CAS: 945554-27-8)], Pigment Red 244 [(CAS: 882858-66-4)], Pigment Red 245 [(CAS: 68016-05-7)], Pigment Red 246 [(CAS: 431991-59-2)], Pigment Red 249 [(CAS: 97955-62-9)], Pigment Red 25 [(CAS: 945426-75-5)], Pigment Red 250 [(CAS: 146358-78-3)], Pigment Red 252 [(CAS: 945554-31-4)], Pigment Red 26 [(CAS: 109944-92-5)], Pigment Red 262 [(CAS: 211502-19-1)], Pigment Red 263 [(CAS: 278792-06-6)], Pigment Red 270 [(CAS: 251086-13-2)], Pigment Red 272 [(CAS: 350249-32-0)], Pigment Red 276 [(CAS: 945554-32-5)], Pigment Red 277 [(CAS: 945554-33-6)], Pigment Red 278 [(CAS: 945554-34-7)], Pigment Red 279 [(CAS: 832743-59-6)], Pigment Red 280 [(CAS: 945554-58-5)], Pigment Red 281 [(CAS: 945554-64-3)], Pigment Red 282 [(CAS: 938065-79-3)], Pigment Red 283 [(CAS: 945554-67-6)], Pigment Red 284 [(CAS: 1089180-60-8)], Pigment Red 285 [(CAS: 1248412-35-2)], Pigment Red 29 [(CAS: 109944-93-6)], Pigment Red 34 [(CAS: 71872-60-1)], Pigment Red 35 [(CAS: 104491-86-3)], Pigment Red 46 [(CAS: 945427-33-8)], Pigment Red 47 [(CAS: 945427-55-4)], Pigment Red 48 [(CAS: 16013-44-8)], Pigment Red 48 [(CAS: 17797-35-2)], Pigment Red 48-Pigment Red 122 mixt. [(CAS: 1046461-81-7)], Pigment Red 48 [(CAS: 218138-44-4)], Pigment Red 48 [(CAS: 218138-41-1)], Pigment Red 48 [(CAS: 68023-17-6)], Pigment Red 51 [(CAS: 25705-30-0)], Pigment Red 51 [(CAS: 446242-29-1)], Pigment Red 52 [(CAS: 27757-95-5)], Pigment Red 52 [(CAS: 67828-72-2)], Pigment Red 52 [(CAS: 218138-27-3)], Pigment Red 53 [(CAS: 15958-19-7)], Pigment Red 56 [(CAS: 25310-96-7)], Pigment Red 57 [(CAS: 88593-07-1)], Pigment Red 58 [(CAS: 25310-97-8)], Pigment Red 59 [(CAS: 945427-99-6)], Pigment Red 60 [(CAS: 446245-60-9)], Pigment Red 63 [(CAS: 5858-84-4)], Pigment Red 63 [(CAS: 16510-21-7)], Pigment Red 63 [(CAS: 1325-13-9)], Pigment Red 64 [(CAS: 5858-77-5)], Pigment Red 68 [(CAS: 25311-19-7)], Pigment Red 71 [(CAS: 384329-78-6)], Pigment Red 72 [(CAS: 945428-03-5)], Pigment Red 73 [(CAS: 109944-94-7)], Pigment Red 74 [(CAS: 109944-95-8)], Pigment Red 75 [(CAS: 109944-96-9)], Pigment Red 78 [(CAS: 71799-06-9)], Pigment Red 81-Pigment White 21 mixt. [(CAS: 192390-71-9)], Pigment Red 82 [(CAS: 110927-51-0)], Pigment Red 88 [(CAS: 14295-43-3)], Pigment Red 90 [(CAS: 51868-24-7)], Pigment Red 92 [(CAS: 909006-04-8)], Pigment Red 94 [(CAS: 12213-62-6)], Pigment Red 96 [(CAS: 945428-04-6)], Pigment Red 97 [(CAS: 239795-92-7)], Pigment Red 98 [(CAS: 945428-07-9)], Pigment Violet 1 [(CAS: 63022-09-3)], Pigment Violet 1 [(CAS: 62973-79-9)], Pigment Violet 11 [(CAS: 875014-31-6)], Pigment Violet 11 [(CAS: 765310-46-1)], Pigment Violet 122 [(CAS: 104491-87-4)], Pigment Violet 123 [(CAS: 80619-33-6)], Pigment Violet 17 [(CAS: 945554-69-8)], Pigment Violet 18 [(CAS: 945554-81-4)], Pigment Violet 21 [(CAS: 945555-53-3)], Pigment Violet 26 [(CAS: 945556-80-9)], Pigment Violet 28 [(CAS: 12236-70-3)], Pigment Violet 30 [(CAS: 12225-07-9)], Pigment Violet 32 [(CAS: 12225-08-0)], Pigment Violet 34 [(CAS: 12612-32-7)], Pigment Violet 35 [(CAS: 55177-94-1)], Pigment Violet 39 [(CAS: 64070-98-0)], Pigment Violet 39 [(CAS: 68477-21-4)], Pigment Violet 4 [(CAS: 68310-88-3)], Pigment Violet 40 [(CAS: 61968-83-0)], Pigment Violet 41 [(CAS: 945557-07-3)], Pigment Violet 42 [(CAS: 71819-79-9)], Pigment Violet 43 [(CAS: 79665-29-5)], Pigment Violet 44 [(CAS: 87209-55-0)], Pigment Violet 45 [(CAS: 945557-40-4)], Pigment Violet 46 [(CAS: 945557-42-6)], Pigment Violet 5 [(CAS: 22297-70-7)], Pigment Violet 50 [(CAS: 76233-81-3)], Pigment Violet 51 [(CAS: 945557-43-7)], Pigment Violet 52 [(CAS: 945557-99-3)], Pigment Violet 53 [(CAS: 945558-15-6)], Pigment Violet 54 [(CAS: 1126076-80-9)], Pigment Violet 55 [(CAS: 1126076-86-5)], Pigment Violet 56 [(CAS: 1126076-93-4)], Pigment Violet 7 [(CAS: 16035-60-2)], Pigment Violet 9 [(CAS: 945554-68-7)], Pigment Yellow 1 [(CAS: 12240-03-8)], Pigment Yellow 102 [(CAS: 12236-74-7)], Pigment Yellow 103 [(CAS: 12225-22-8)], Pigment Yellow 106 [(CAS: 12225-23-9)], Pigment Yellow 107 [(CAS: 12270-64-3)], Pigment Yellow 113 [(CAS: 14359-20-7)], Pigment Yellow 120 [(CAS: 29920-31-8)], Pigment Yellow 121 [(CAS: 14569-54-1)], Pigment Yellow 122 [(CAS: 852620-87-2)], Pigment Yellow 125 [(CAS: 304891-45-0)], Pigment Yellow 131 [(CAS: 945423-41-6)], Pigment Yellow 132 [(CAS: 945424-04-4)], Pigment Yellow 135 [(CAS: 945424-77-1)], Pigment Yellow 136 [(CAS: 181285-33-6)], Pigment Yellow 140 [(CAS: 945425-58-1)], Pigment Yellow 141 [(CAS: 945425-59-2)], Pigment Yellow 142 [(CAS: 177020-91-6)], Pigment Yellow 143 [(CAS: 945425-60-5)], Pigment Yellow 144 [(CAS: 945425-61-6)], Pigment Yellow 145 [(CAS: 115742-72-8)], Pigment Yellow 146 [(CAS: 945425-66-1)], Pigment Yellow 149 [(CAS: 945425-67-2)], Pigment Yellow 150 [(CAS: 939382-97-5)], Pigment Yellow 151 [(CAS: 31837-42-0)], Pigment Yellow 154 [(CAS: 68134-22-5)], Pigment Yellow 156 [(CAS: 63661-26-7)], Pigment Yellow 165 [(CAS: 865763-85-5)], Pigment Yellow 166 [(CAS: 76233-82-4)], Pigment Yellow 170 [(CAS: 31775-16-3)], Pigment Yellow 171 [(CAS: 53815-04-6)], Pigment Yellow 172 [(CAS: 76233-80-2)], Pigment Yellow 175 [(CAS: 35636-63-6)], Pigment Yellow 178 [(CAS: 945425-73-0)], Pigment Yellow 17 [(CAS: 221358-38-9)], Pigment Yellow 18 [(CAS: 1326-11-0)], Pigment Yellow 18 [(CAS: 68310-89-4)], Pigment Yellow 186 [(CAS: 945425-92-3)], Pigment Yellow 187 [(CAS: 131439-24-2)], Pigment Yellow 189 [(CAS: 69011-05-8)], Pigment Yellow 191 [(CAS: 1051932-58-1)], Pigment Yellow 195 [(CAS: 135668-58-5)], Pigment Yellow 196 [(CAS: 945425-96-7)], Pigment Yellow 197 [(CAS: 945425-97-8)], Pigment Yellow 198 [(CAS: 516493-10-0)], Pigment Yellow 20 [(CAS: 61512-63-8)], Pigment Yellow 200 [(CAS: 945425-98-9)], Pigment Yellow 201 [(CAS: 945425-99-0)], Pigment Yellow 204 [(CAS: 945426-05-1)], Pigment Yellow 205 [(CAS: 945426-18-6)], Pigment Yellow 206 [(CAS: 945426-19-7)], Pigment Yellow 207 [(CAS: 945426-23-3)], Pigment Yellow 208 [(CAS: 945426-25-5)], Pigment Yellow 209 [(CAS: 945426-27-7)], Pigment Yellow 21 [(CAS: 945421-49-8)], Pigment Yellow 210 [(CAS: 945426-35-7)], Pigment Yellow 211 [(CAS: 945426-36-8)], Pigment Yellow 212 [(CAS: 945426-37-9)], Pigment Yellow 214 [(CAS: 577980-23-5)], Pigment Yellow 215 [(CAS: 913621-26-8)], Pigment Yellow 216 [(CAS: 817181-98-9)], Pigment Yellow 217 [(CAS: 945426-39-1)], Pigment Yellow 219 [(CAS: 874963-72-1)], Pigment Yellow 221 [(CAS: 945426-41-5)], Pigment Yellow 223 [(CAS: 2095507-47-2)], Pigment Yellow 224 [(CAS: 1207669-05-3)], Pigment Yellow 23 [(CAS: 4981-43-5)], Pigment Yellow 231 [(CAS: 2148300-50-7)], Pigment Yellow 25 [(CAS: 945421-63-6)], Pigment Yellow 26 [(CAS: 945421-64-7)], Pigment Yellow 27 [(CAS: 945421-65-8)], Pigment Yellow 28 [(CAS: 945421-66-9)], Pigment Yellow 29 [(CAS: 945421-67-0)], Pigment Yellow 34 [(CAS: 147858-25-1)], Pigment Yellow 36 [(CAS: 37300-23-5)], Pigment Yellow 37 [(CAS: 68859-25-6)], Pigment Yellow 40 [(CAS: 13782-01-9)], Pigment Yellow 47 [(CAS: 12060-00-3)], Pigment Yellow 50 [(CAS: 945421-71-6)], Pigment Yellow 51 [(CAS: 945421-76-1)], Pigment Yellow 56 [(CAS: 12225-09-1)], Pigment Yellow 58 [(CAS: 12225-11-5)], Pigment Yellow 61 [(CAS: 12286-65-6)], Pigment Yellow 72 [(CAS: 945421-81-8)], Pigment Yellow 79 [(CAS: 331414-25-6)], Pigment Yellow 8 [(CAS: 71872-65-6)], Pigment Yellow 80 [(CAS: 945421-85-2)], Pigment Yellow 82 [(CAS: 12225-14-8)], Pigment Yellow 84 [(CAS: 945421-87-4)], Pigment Yellow 85 [(CAS: 12286-67-8)], Pigment Yellow 86 [(CAS: 12286-68-9)], Pigment Yellow 86 [(CAS: 5280-65-9)], Pigment Yellow 88 [(CAS: 945422-67-3)], Pigment Yellow 89 [(CAS: 945422-85-5)], Pigment Yellow 90 [(CAS: 713104-87-1)], Pigment Yellow 91 [(CAS: 945423-18-7)], Pigment Yellow 96 [(CAS: 12213-63-7)], Pigment Yellow 97 [(CAS: 12225-18-2)], Pigment Yellow 99 [(CAS: 12225-20-6)]

The pigment(s) used in the composition according to the coloring embodiments of the present disclosure can include at least two different pigments selected from the above pigment group, or can include at least three different pigments selected from the above pigment group. According to an embodiment, the pigment(s) used in the composition can include at least one yellow pigment selected from the yellow pigment group consisting of: a Pigment Yellow 83 (CI 21108), CAS #5567-15-7, Pigment Yellow 155 (C.I. 200310), (CAS: 68516-73-4), Pigment Yellow 180 (C.I. 21290), (CAS: 77804-81-0).

In addition to the at least one yellow pigment, or alternatively, the pigments(s) used in the composition can include at least one red pigment selected from the red pigment group consisting of: Pigment Red 5 (CI 12490), (CAS #6410-41-9), Pigment Red 112 (CI 12370), (CAS #6535-46-2), Pigment Red 122 (CI 73915), (CAS #980-26-7).

In addition to the at least one yellow pigment and/or the at least one red pigment, or alternatively, the pigments(s) used in the composition can include at least one green pigment selected from the green pigment group consisting of: Pigment Green 36, (C.I. 74265), (CAS: 14302-13-7).

In addition to the at least one yellow pigment and/or the at least one red pigment and or the at least one green pigment, or alternatively, the pigments(s) used in the composition can include at least one blue pigment selected from the blue pigment group consisting of: Pigment Blue 16, (CAS: 424827-05-4), Pigment Blue 60 (C.I. 69800), (CAS: 81-77-6), Pigment Blue 66, (C.I. 73000), (CAS: 482-89-3)

In addition to the at least one yellow pigment and/or the at least one red pigment and/or the at least one green pigment, and/or the at least one blue pigment or alternatively, the pigments(s) used in the composition can include at least one black pigment selected from the black pigment group consisting of: Pigment Black 6 (C.I. 77266), (CAS 1333-86-4), Pigment Black 7 (C.I. 77266), (CAS 1333-86-4).

In addition to the at least one yellow pigment and/or the at least one red pigment and/or the at least one green pigment, and/or the at least one blue pigment, and/or the at least one black pigment or alternatively, the pigments(s) used in the composition can include at least one pigment selected from Pigment White 6, Pigment Violet 19.

The pigment(s) can optionally have a surface zeta potential of ≥±15 mV, preferably ≥±20 mV, more preferably ≥±25 mV. The surface zeta potential can be measured with a zetasizer, for example, a Zetasizer 3000 HS. Surface zeta potential measurements are conducted, for example, according to ISO 13099.

For example, the white or colored organic pigments can be chosen from carmine, carbon black, aniline black, melanin, azo yellow, quinacridone, phthalocyanin blue, sorghum red, the blue pigments codified in the Color Index under the references CI 42090, 69800, 69825, 73000, 74100, and 74160, the yellow pigments codified in the Color Index under the references CI 11680, 11710, 15985, 19140, 20040, 21090, 21100, 21108, 47000, 47005 and 77492.

The green pigments codified in the Color Index under the references CI 61565, 61570, 74265, and 74260, the orange pigments codified in the Color Index under the references CI 11725,12075, 15510, 45370, and 71105, the red pigments codified in the Color Index under the references CI 12085, 12120, 12370, 12420, 12490, 14700, 15525, 15580, 15585, 15620, 15630, 15800, 15850, 15865, 15880, 17200, 26100, 45380, 45410, 45430, 58000, 73360, 73915, 75470, and 77491 and the pigments obtained by oxidative polymerization of indole or phenolic derivatives as described in French Patent Publication No. FR 2 679 771, which is incorporated herein by reference.

Non-limiting examples that can also be mentioned include pigmentary pastes of organic pigments, such as the products sold by the company Hoechst under the names: JAUNE COSMENYL IOG: Pigment Yellow 3 (CI 11710); JAUNE COSMENYL G: Pigment Yellow 1 (CI 11680); ORANGE COSMENYL GR: Pigment Orange 43 (CI 71105); ROUGE COSMENYL R: Pigment Red 4 (CI 12085); CARMINE COSMENYL FB: Pigment Red 5 (CI 12490); VIOLET COSMENYL RL: Pigment Violet 23 (CI 51319); BLEU COSMENYL A2R: Pigment Blue 15.1 (CI 74160); VERT COSMENYL GG: Pigment Green 7 (CI 74260); and NOIR COSMENYL R: Pigment Black 7 (CI 77266).

The at least one pigment in accordance with the present disclosure can also be in the form of at least one composite pigment as described in European Patent Publication No. EP 1 184 426 A2. These composite pigments can be, for example, compounds of particles comprising a mineral core, at least one binder for ensuring the binding of the organic pigments to the core, and at least one organic pigment at least partially covering the core.

The at least one pigment in accordance with the present disclosure can be in the form of small undissolved microparticles, which do not diffuse into the hair color, but deposit on the outer wall of the keratin fiber. Suitable color pigments can be of organic and/or inorganic origin. But the pigments can also be inorganic color pigments, given the excellent light, weather and/or temperature resistance thereof.

Inorganic pigments, whether natural or synthetic in origin, include those produced from chalk, red ocher, umbra, green earth, burnt sienna or graphite, for example. Furthermore, it is possible to use black pigments, such as iron oxide black, color pigments such as ultramarine or iron oxide red, and fluorescent or phosphorescent pigments as inorganic color pigments.

Colored metal oxides, metal hydroxides and metal oxide hydrates, mixed phase pigments, sulfurous silicates, silicates, metal sulfides, complex metal cyanides, metal sulfates, metal chromates and/or metal molybdates are particularly suitable. In particular, preferred color pigments are black iron oxide (Cl 77499), yellow iron oxide (Cl 77492), red and brown iron oxide (Cl 77491), manganese violet (Cl 77742), ultramarine (sodium aluminum sulfosilicates, Cl 77007, Pigment Blue 29), chromium oxide hydrate (CI 77289), iron blue (ferric ferrocyanide, CI 77510) and/or carmine (cochineal).

The at least one pigment can also be colored pearlescent pigments. These are usually mica-based and can be coated with one or more metal oxides from the group consisting of titanium dioxide (CI 77891), black iron oxide (CI 77499), yellow iron oxide (CI 77492), red and brown iron oxide (Cl 77491, CI 77499), manganese violet (Cl 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Mica forms part of the phyllosilicates, including muscovite, phlogopite, paragonite, biotite, lepidolite, and margarite. To produce the pearlescent pigments in combination with metal oxides, the mica, primarily muscovite or phlogopite, is coated with a metal oxide.

As an alternative to natural mica, it is also optionally possible to use synthetic mica coated with one or more metal oxides as the pearlescent pigment. Such suitable pearlescent pigments based on natural micas are described in, e.g., WO 2005/065632. The at least one pigment can also be pearlescent pigments based on natural or synthetic mica and are coated with one or more of the aforementioned metal oxides. The color of the respective pigments can be varied by varying the layer thickness of the metal oxide or metal oxides.

The at least one pigment can also be at least one inorganic color pigment selected from the group consisting of colored metal oxides, metal hydroxides, metal oxide hydrates, silicates, metal sulfides, complex metal cyanides, metal sulfates, bronze pigments and/or colored pigments based on mica, which are coated with at least one metal oxide and/or a metal oxychloride.

The at least one pigment can also be at least one mica-based colored pigment, which is coated with one or more metal oxides from the group consisting of titanium dioxide (CI 77891), black iron oxide (CI 77499), yellow iron oxide (CI 77492), red and/or brown iron oxide (Cl 77491, CI 77499), manganese violet (Cl 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

The at least one pigment can also be color pigments commercially available, for example, under the trade names Rona®, Colorona®, Dichrona® and Timiron® from Merck, Ariabel® and Unipure® from Sensient, Prestige® from Eckart Cosmetic Colors, and Sunshine® from Sunstar.

The at least one pigment can also be color pigments bearing the trade name Colorona® are, for example: Colorona Copper, Merck, MICA, Cl 77491 (IRON OXIDES); Colorona Passion Orange, Merck, Mica, Cl 77491 (Iron Oxides), Alumina; Colorona Patina Silver, Merck, MICA, Cl 77499 (IRON OXIDES), Cl 77891 (TITANIUM DIOXIDE); Colorona RY, Merck, Cl 77891 (TITANIUM DIOXIDE), MICA, Cl 75470 (CARMINE); Colorona Oriental Beige, Merck, MICA, Cl 77891 (TITANIUM DIOXIDE), Cl 77491 (IRON OXIDES); Colorona Dark Blue, Merck, MICA, TITANIUM DIOXIDE, FERRIC FERROCYANIDE; Colorona Chameleon, Merck, Cl 77491 (IRON OXIDES), MICA; Colorona Aborigine Amber, Merck, MICA, Cl 77499 (IRON OXIDES), Cl 77891 (TITANIUM DIOXIDE); Colorona Blackstar Blue, Merck, Cl 77499 (IRON OXIDES), MICA; Colorona Patagonian Purple, Merck, MICA, Cl 77491 (IRON OXIDES), Cl 77891 (TITANIUM DIOXIDE), Cl 77510 (FERRIC FERROCYANIDE); Colorona Red Brown, Merck, MICA, Cl 77491 (IRON OXIDES), Cl 77891 (TITANIUM DIOXIDE); Colorona Russet, Merck, C1 77491 (TITANIUM DIOXIDE), MICA, Cl 77891 (IRON OXIDES); Colorona Imperial Red, Merck, MICA, TITANIUM DIOXIDE (Cl 77891), D&C RED NO. 30 (Cl 73360); Colorona Majestic Green, Merck, Cl 77891 (TITANIUM DIOXIDE), MICA, Cl 77288 (CHROMIUM OXIDE GREENS); Colorona Light Blue, Merck, MICA, TITANIUM DIOXIDE (Cl 77891), FERRIC FERROCYANIDE (Cl 77510); Colorona Red Gold, Merck, MICA, Cl 77891 (TITANIUM DIOXIDE), Cl 77491 (IRON); Colorona Gold Plus MP 25, Merck, MICA, TITANIUM DIOXIDE (Cl 77891), IRON OXIDES (Cl 77491); Colorona Carmine Red, Merck, MICA, TITANIUM DIOXIDE, CARMINE Colorona Blackstar Green, Merck, MICA, Cl 77499 (IRON OXIDES); Colorona Bordeaux, Merck, MICA, Cl 77491 (IRON OXIDES); Colorona Bronze, Merck, MICA, C1 77491 (IRON OXIDES); Colorona Bronze Fine, Merck, MICA, Cl 77491 (IRON OXIDES); Colorona Fine Gold MP 20, Merck, MICA, Cl 77891 (TITANIUM DIOXIDE), Cl 77491 (IRON OXIDES); Colorona Sienna Fine, Merck, Cl 77491 (IRON OXIDES), MICA Colorona Sienna, Merck, MICA, Cl 77491 (IRON OXIDES); Colorona Precious Gold, Merck, Mica, Cl 77891 (Titanium dioxide), Silica, Cl 77491 (Iron oxides), Tin oxide; Colorona Sun Gold Sparkle MP 29, Merck, MICA, TITANIUM DIOXIDE, IRON OXIDES, MICA, Cl 77891, Cl 77491 (EU); Colorona Mica Black, Merck, Cl 77499 (Iron oxides), Mica, Cl 77891 (Titanium dioxide) Colorona Bright Gold, Merck, Mica, Cl 77891 (Titanium dioxide), Cl 77491 (Iron oxides); Colorona Blackstar Gold, Merck, MICA, Cl 77499 (IRON OXIDES); color pigments bearing the trade name Unipure® are, for example: Unipure Red LC 381 EM, Sensient Cl 77491 (Iron Oxides), Silica; Unipure Black LC 989 EM, Sensient, Cl 77499 (Iron Oxides), Silica; Unipure Yellow LC 182 EM, Sensient, Cl 77492 (Iron Oxides), Silica.

The at least one pigment can also include particles of gold, silver, copper, brass, aluminum, or a combination thereof. Alternatively or in addition to the particles, the at least one pigment can also include flakes of gold, silver, copper, brass, aluminum, or a combination thereof.

Depending on the degree of the change in color that is desired on the keratin fiber, the at least one pigment can also be can be used in varying amounts. The more color pigment that is used, the higher is the extent of the change in color in general. Starting at a certain usage amount, however, the adherence of the pigments to the keratin fiber approaches a limiting value, beyond which it is no longer possible to increase the extent of the change in color by further increasing the pigment amount used. While not wishing to be bound by any specific theory, it is believed that when a certain thickness is achieved, an insignificant amount of the incident lights passes through the pigment layer to make a difference to the observed color due to the hair itself. The rest of the light is either scattered back towards the surface, or absorbed.

The at least one pigment can be partially (Scheme 1, (b), where the dark oval represents a pigment, even though the pigment can be white or colorless) or completely enveloped in a matrix (e.g., a polymer matrix or an inorganic matrix; (Scheme 1, (a)). Or the pigment can be adhered to the surface of a matrix that can be colored or colorless (Scheme 1 (c)).

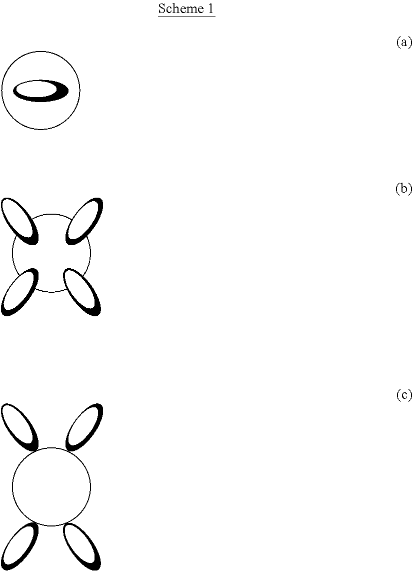

The matrix can be, e.g., CaCO₃, MnCO₃. Or the matrix can be a melamine formaldehyde matrix.

In another example, the at least one pigment can be encapsulated in silica, as described in Published U.S. Appl. No. 2007/0134180. Other examples of encapsulated pigments include encapsulated Carmine, Iron Oxides, Titanium dioxide, and Chrome Oxide/Hydroxide, the colorants D&C Red 21 Aluminum Lake, D&C Red 7 Calcium Lake, D&C Green 6 Liposoluble, and Aluminium Blue #1 (Indigo Carmine Lake). The encapsulated pigment can be titanium dioxide (used to lighten other pigments and to lend opacity to formulations) in any one of its mineral forms anatase, brookite or rutile, or mixtures thereof. Or the pigment can be at least one iron oxide in any of the 3 basic colors—red, black and yellow iron oxides, or mixtures thereof. From these 3 oxides and the addition of titanium dioxide, any shade of brown (skin tones) can be achieved.

The organic pigment can also be a lake. As used herein, the term “lake” means at least one dye adsorbed onto insoluble particles, the assembly thus obtained remaining insoluble during use. The inorganic substrates onto which the dyes are adsorbed can be, for example, alumina, silica, calcium sodium borosilicate, calcium aluminum borosilicate, calcium carbonate, manganese carbonate, aluminum, nitro-dyes, triarylmethin dyes, Azo-dyes, Anthrazen, Acid dyes, polymethine dyes, triarylmethin dyes, aza annulene dyes and polymethine dyes.

Among the dyes, non-limiting mention can be made of cochineal carmine. Non-limiting mention can also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 0 (CI 77 002), D&C Green 3 (CI 42 053), and D&C Blue 1 (CI 42 090). A non-limiting example of a lake that can be mentioned is the product known under the following name: D&C Red 7 (CI 15 850:1).

The at least one pigment can also be a pigment with special effects. As used herein, the term “pigments with special effects” means pigments that generally create a non-uniform colored appearance (characterized by a certain shade, a certain vivacity, and a certain lightness) that changes as a function of the conditions of observation (light, temperature, observation angles, etc.). They thus contrast with white or colored pigments that afford a standard uniform opaque, semi-transparent, or transparent shade.

Several types of pigments with special effects exist, including those with a low refractive index, such as fluorescent, photochromic, or thermochromic pigments, and those with a high refractive index, such as nacres or glitter flakes. Examples of pigments with special effects of which non-limiting mention can be made include nacreous pigments such as mica coated with titanium or with bismuth oxychloride, colored nacreous pigments such as titanium mica with iron oxides, titanium mica for example with ferric blue or with chromium oxide, titanium mica with an organic pigment of the abovementioned type, and also nacreous pigments based on bismuth oxychloride. Nacreous pigments of which non-limiting mention can be made include the CELLINI nacres sold by Engelhard (mica-TiO₂-lake), PRESTIGE sold by Eckart (mica-TiO₂), PRESTIGE BRONZE sold by Eckart (mica-Fe₂O₃), and COLORONA sold by Merck (mica-TiO₂—Fe₂O₃).

In addition to nacres on a mica support, multilayer pigments based on synthetic substrates such as alumina, silica, sodium calcium borosilicate, calcium aluminum borosilicate, and aluminum, can be envisaged.

Non-limiting mention can also be made of pigments with an interference effect that are not fixed onto a substrate, for instance liquid crystals (HELICONES HC from Wacker) and holographic interference flakes (GEOMETRIC PIGMENTS or SPECTRA F/X from Spectratek). Pigments with special effects also comprise fluorescent pigments, whether these are substances that are fluorescent in daylight or that produce an ultraviolet fluorescence, phosphorescent pigments, photochromic pigments, thermochromic pigments, and quantum dots, sold, for example, by the company Quantum Dots Corporation.

Quantum dots are luminescent semiconductive nanoparticles capable of emitting, under light excitation, irradiation with a wavelength ranging from 400 nm to 700 nm. These nanoparticles are known from the literature. They can be manufactured, for example, according to the processes described, for example, in U.S. Pat. Nos. 6,225,198 or 5,990,479 which are incorporated herein by reference, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997 pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029.

The variety of pigments that can be used in the present disclosure makes it possible to obtain a wide range of colors, and also optical effects such as metallic effects or interference effects.

The pigments that can be used in the present disclosure can transmit light of various wavelengths, including visible light (e.g., light having a wavelength of above 350 nm). The pigment(s) can also transmit light of certain wavelengths, but also reflect light of certain wavelengths. And the pigment(s) can also be 100% reflective. For examples, reflective pigments provide a high specular reflection of visible light. Reflective pigments include those that are partially or completely coated with a non-matt and non-scattering surface layer of a metal or metal oxide. The substrate can be chosen from glasses, ceramics, graphite, metal oxides, aluminas, silicas, silicates, especially aluminosilicates and borosilicates and synthetic mica (e.g., fluorophlogopite), to name a few. The metal or metal oxide can be, without limitation, titanium oxides, iron oxides, tin oxide, chromium oxide, barium sulfate, MgF₂, CeF₃, ZnS, ZnSe, SiO₂, Al₂O₃, MgO, Y₂O₃, SeO₃, SiO, HfO₂, ZrO₂, CeO₂, Nb₂O₅, Ta₂O₅ and MoS₂, and mixtures thereof. Reflective pigments can have a spectral reflectance in the visible spectrum of at least 70%.

Other reflective pigments include those having non-goniochromatic layered structure of two or more polymeric and/or metallic layers of different refractive indices. For example, reflective particles comprising layers of 2,6-polyethylene naphthalate (PEN) and of polymethyl (meth)acrylate are sold by 3M under the name Mirror Glitter™. Other effect pigments are available under the trade name Metasomes Standard/Glitter in various colors (yellow, red, green, blue) from Flora Tech.

Color Gamut for Pigment Blends

CIE L*a*b* (CIELAB) is a color space specified by the International Commission on Illumination. It describes all the colors visible to the human eye and serves as a device-independent model to be used as a reference.

The three coordinates of CIELAB represent the lightness of the color (L*=0 yields black and L*=100 indicates diffuse white; specular white may be higher), its position between red/magenta and green (a*, negative values indicate green while positive values indicate magenta) and its position between yellow and blue (b*, negative values indicate blue and positive values indicate yellow).

Since the L*a*b* model is a three-dimensional model, it can be represented properly only in a three-dimensional space. Two-dimensional depictions include chromaticity diagrams: sections of the color solid with a fixed lightness.

Because the red-green and yellow-blue opponent channels are computed as differences of lightness transformations of (putative) cone responses, CIELAB is a chromatic value color space.

In the present invention, the color gamut is determined by adding each pigment to be tested in the hair coloring composition, and then individually tested at a level such that when applied to hair, the resulting CIELAB lightness or L* value of the colored hair is 60±2. The level of pigment needed will depend on the pigment being tested. Two hair tresses (Kerling, Natural White special quality) have the composition applied as described herein. A Minolta spectrophotometer CM-2600d is used to measure the color of the dried hair tresses, five points on both the front and back sides, and the values averaged. The D65 L*a*b values are calculated. When at least three pigments have each been measured such that their resulting color reside within the target L* values of 60±2 the color gamut can be calculated. First the lengths of each side of the resulting triangle of each combination of three pigments in the a*b plane are computed using the following expressions. To calculate the distance between pigments 1 and pigment 2 the following equation is used:

Side LengthSL₁₂=((a_{pigment 1}−a_{pigment 2})²+(b_{pigment 1}−b_{pigment 2})²)^{0.5}.

This is computed for each pair of pigments. Then for a series of three pigments.

The resulting color gamut is calculated using the expression:

Color Gamut=(S(S−SL₁₂)(S−SL₁₃)(S−SL₂₃))^{0.5}
wherein SL₁₂, SL₁₃, and SL₂₃ are the three lengths of the sides of the triangle within the a*b plane, and S=(SL₁₂+SL₁₃+SL₂₃)/2. Where more than three pigments are used, this calculation can be performed for each combination of the three pigment from the more than three pigments used, and the largest Color Gamut is selected.

The hair coloring composition embodiments of the present invention can also have a color gamut of greater than 250, greater than 500, greater than 750, greater than 800, greater than 900, greater than 1100 or even greater than 1250.

Using the above expression, for each combination of three pigments possible from Color Gamut Tables 1, as illustrated below, the color gamut at a nominal L value of 60 was calculated

Color Gamut Table 1:

| | | | wt % | | | |
|---|---|---|---|---|---|---|
| Pigment | Name | Supplier | level | L | a | b |
| Blue 15 | PV Fast Blue BG-NIP | Clariant | 0.155 | 59.3 | −18.7 | −2.1 |
| Blue 16 | Phthalocyanine | Carbosynth | 0.280 | 59.4 | −17.3 | 1.5 |
| Blue 66 | Indigo 229296 | Aldrich | 0.105 | 60.0 | −3.1 | 6.8 |
| Blue 60 | Paliogen Blau L 6482 | BASF | 0.260 | 60.7 | −3.9 | 5.9 |
| Black 7 | Midnight Black | Geotech | 0.045 | 59.8 | 0.0 | 12.3 |
| Green 36 | Heliogen Green K 9362 | BASF | 0.509 | 60.1 | −32.8 | 20.2 |
| Red 112 | Permanent Red FGR 250 | Clariant | 0.150 | 60.1 | 29.8 | 18.8 |
| Red 122 | Hostaperm Pink E02- | Clariant | 0.140 | 59.5 | 24.9 | 6.1 |
| | EDW VP4034 | | | | | |
| Violet 19 | Ink Jet Magenta E5B 02 | Clariant | 0.200 | 60.6 | 28.1 | 10.1 |
| | M250 | | | | | |
| Red 5 | Permanent Carmine FB01 | Clariant | 0.140 | 59.7 | 30.1 | 14.4 |
| Yellow 155 | Ink Jet Yellow 4GC | Clariant | 16.92 | 61.8 | 9.6 | 74.4 |
| Yellow 83 | Novoperm Yellow HR 70 | Clariant | 1.059 | 60.0 | 12.5 | 61.8 |
| Yellow 180 | Toner Yellow HG | Clariant | 9.16 | 61.4 | 11.2 | 72.8 |

### E Dispersants

It will be apparent to one skilled in the art that careful and selective choice of dispersant can help to maximize performance in terms of maximizing the amount of color produced, maximizing the remanence or washfastness, and enabling removal of the color. Generally, according to the present disclosure, the pigments may be used without relying on dispersants. When dispersants are used, it is preferred using dispersants which are bio-based. According to embodiments, dispersants used in the composition of the present invention may be derived from renewable resources, are biodegradable as defined herein. In particular embodiments, dispersants used in the composition of the present disclosure are both derived from renewable resources and are biodegradable as defined herein.

In the case where the bio-based polymer is anionic in nature, dispersants which are anionic or nonionic preferably may be chosen, rather than cationic, as this avoids undesired precipitation in the formula prior to it forming a colored film on the keratin—i.e. utilizing the principle of avoiding opposing charges.

As well as compatibility as noted above, the other critical criterion in selecting dispersant(s) is their ability to enable pigment to be dispersed down to the primary particle size, preferably with the minimum amount of input mechanical energy. It will be recognized by someone skilled in the art that the concentration of dispersing agent is also a critical factor. In general, it is usually required that there is a minimum amount for dispersing activity and that below this, the system is either not fully dispersed or, worse, that the dispersant acts as a flocculant.

These two considerations together are used to define preferred materials and their respective concentrations.

It may also be the case, depending on the type of the bio-based polymer used, that the bio-based polymer itself is also a dispersant (see below for discussion of classes of dispersant). In such cases it is possible that no further dispersing additive may be needed.

### E1 Overview of Dispersant Kinds, Properties and Chemistry

Dispersants are amphiphilic or amphiphathic meaning that they are chemical compounds possessing both hydrophilic (water-loving, polar) and lipophilic (fat-loving) properties. Dispersants are surface-active polymers that allow the homogeneous distribution and stabilization of solids, e.g. pigments in a liquid medium, by lowering the interfacial tension between the two components. As a result, agglomerates are broken up into primary particles and protected by a protecting dispersant envelope of a re-agglomeration.

The dispersants can be subdivided on the basis of the stabilization mechanism in
1. Dispersants for electrostatic stabilization
   a. Anionic dispersing additives
      i. Polyacrylates
      ii. Polyphosphates
   b. Neutral dispersing additives
   c. Cationic dispersing additives
2. Dispersants for steric stabilization

### E2 Electrostatic Stabilization

The pigment surface is occupied by an additive carrying an ionic charge. All pigment particles are charged the same. The mutual repulsion by the charge is greater than the attractions of the pigment particles. The electrostatic stabilization has its relevance mostly in water-based paint systems.
Polyanionic dispersing additives: polycarboxylates (mostly salts of polyacrylic acids), polyphosphates divided into linear polyphosphates and cyclic metaphosphates, polyacrylates
salts of polyacrylic acid, as cations, sodium and ammonium are preferred, these polyacrylates are water-soluble, technical products have molecular weights in the range of 2000 to 20,000 g/mol, optimum is about 8000 g/mol
Sodium and ammonium salts of the homo- or copolymers of acrylic acid, methacrylic acid or maleic acid

### E3 Steric Stabilization

The attractive forces between the pigment particles are effective only over relatively small distances of the particles from each other. The approach of two particles to each other can be prevented by molecules that are firmly anchored to the pigment surface and carry groups that extend from the surface and may reduce the potential for the pigments to contact one another. By sufficiently long chain lengths, agglomeration can be prevented.
Water-soluble polymers
Block or graft copolymers, so-called AB block copolymers
Example: AB block polymer of 2-vinylpyridine and methacrylic acid ester
Example: AB block copolymer of polyester (based caprolactam) and triethylenetetramine
Typical functional groups for the A segment are carboxyl, amine, sulfate and phosphate for inogenous bonds or polyether and polyamide for hydrogen bonds. B represents the solvated side chain, molecular weights 1000 to 15000 g/mol, e.g. modified polyacrylates or polyhydroxystearates
Hydrophilic moieties (e.g., polyethers) and pigment affinic groups (e.g. Groups) containing oligomers or polymers.

The following types are distinguished according to the number of monomer types used in the production:
Homopolymers: only one kind of monomer
Copolymers: two monomers
Terpolymers: three monomers

Classification according to distribution of the monomers in the polymer:
Statistical polymers: A and B segments are distributed arbitrarily
Block polymers: the monomers are grouped into blocks
Graft polymers: these consist of a linear homopolymer backbone on which side chains of other monomer blocks are grafted

### E4 Dispersant Materials

Some examples of dispersants for solvent-based systems are:
Oligomeric titanates and silanes for inorganic pigments with OH or carboxy groups.
Oligomeric polymeric carboxylic acids for inorganic pigments (cationic).
Polyamines for inorganic pigments, e.g., cationic polymers.
Salts of long-chain polyamines and polycarboxylic acids for inorganic and organic pigments (electroneutral).
Amine/amide-functional polyesters/polyacrylates for the stabilization of organic pigments.
Polysilicones with and without functional groups including cyclic siloxanes, amine functional cyclic and linear siloxanes, carboxyl functional cyclic and linear siloxanes.

Some examples of dispersants for aqueous systems are:
Inorganic dispersants such as fine-grained CaCO3, Ca3 (PO4) 2, polyphosphates, polyphosphoric acids.
Nonionic surfactants such as ethoxlyated fatty alcohol (e.g. Neodol 25-9), ethoxylated oils (e.g. ethxylated castor oil under the tradename Cremophore RH410)
Block and graft copolymers of the type having distinct hydrophilic and hydrophobic blocks (e.g. ethylene oxide—propylene oxide polymers under the tradename Poloxamer)
Anionic surfactants consisting of the unethoxylated or ethoxylated salts of acids (e.g. sodium ceteth-10-phosphate under the tradename Crodafos).

Examples and classes of nonionic surfactants that can function as dispersants include oligomers (e.g., example, oligomers have up to 20 monomeric units, polymers have at least 20 monomeric units), polymers, and/or a mixture of several thereof, bearing at least one functional group with strong affinity for the surface of the pigment microparticles. For example, they can physically or chemically attach to the surface of the pigment microparticles. These dispersants also contain at least one functional group that is compatible with or soluble in the continuous medium. For example, 12-hydroxystearic acid esters and C8 to C20 fatty acid esters of polyols such as glycerol or diglycerol are used, such as poly(12-hydroxystearic acid) stearate with a molecular weight of about 750 g/mol, such as the product sold under the name SOLSPERSE 21,000 by the company Avecia, polyglyceryl-2 dipolyhydroxystearate (CTFA name) sold under the reference DEHYMYLS PGPH by the company Henkel, or polyhydroxystearic acid such as the product sold under the reference ARLACEL P100 by the company Uniqema, and mixtures thereof.

The foregoing dispersant category involving cationic polymers includes polymers such as quaternary ammonium polymers. Examples of quaternary ammonium derivatives of polycondensed fatty acids include, such as for instance, SOLSPERSE 17,000 sold by the company Avecia, and polydimethylsiloxane/oxypropylene mixtures such as those sold by the company Dow Corning under the references DC2-5185 and DC2-5225 C.

The dispersant can be a polyolefin polymer. These dispersants include but are not limited to an olefinic polymer having a molecular weight of about 100 g/mol to about 5,000,000 g/mol, such as about 1,000 g/mol to about 1,000,000 g/mol. Examples of polymers, include, but are not limited to poly(ethylene), poly(propylene), poly(butylene), poly(isobutylene), poly(isoprene), poly(acetal), poly(ethylene glycol), poly(propylene glycol), poly(butylene glycol), poly(methylmethacrylate), poly(dimethylsiloxane), poly(vinylalcohol), poly(styrene), poly(maleic anhydride), poly(ethylmethacrylate), poly(isobutylmethacrylate), poly(methacrylate), poly(butylmethacrylate), poly(n-butylmethacrylate), poly(vinyl butyrate), poly(vinyl chloride), polysiloxane, and mixtures thereof. The polymers can be random, block, or alternating copolymers. In some embodiments, the polymer is a co-polymer that is made from two or more different monomers, such as the monomers that make the polymers described above. Examples of co-polymers include, but are not limited to polyethers, polyesters, polyamides, acrylics, and polystyrenes. The co-polymer can be alternating monomers, random, or block. Examples include a polyether of alternating or block PEO, PPO groups. Examples of acidic groups include, but are not limited to, carboxylic acids, sulfinic acids, sulfonic acids, phosphonic acids, phosphate esters, maleic anhydrides, and succinic anhydride. In some embodiments, the dispersive additive comprises a group selected from phosphonate, phosphate, phosphite, phosphine, and phosphate ester, such as a phosphate, phosphite, and phosphonic acid. In some embodiments, the acidic group has been converted into a salt.

Representative dispersants are also available from a variety of suppliers, and include various nonionic (e.g., ethoxylated) and anionic (e.g., non-ethoxylated salt) forms including agents from Air Products and Chemicals, Inc. (e.g., SURFYNOL™ PSA336); Archer Daniels Midland Co. (e.g., ULTRALEC™ F deoiled lecithin); Ashland Inc. (e.g., NEKAL™ WS-25-I, which is a sodium bis(2,6-dimethyl 4heptyl)sulfosuccinate); BASF (e.g., DISPEX™ AA 4144, DISPEX ULTRA FA 4425 which is a fatty acid-modified emulsifier having a viscosity of 40,000 cps, DISPEX ULTRA FA 4420 which is a fatty acid-modified emulsifier and a dark brown liquid of unspecified viscosity, DISPEX ULTRA FA 4431 which is an aliphatic polyether with acidic groups having a viscosity of 350 cps, DISPEX ULTRA PA 4501 which is a fatty acid modified polymer having a viscosity of 10,000 cps, DISPEX ULTRA PA 4510, EFKA™ PU 4010, EFKA PU 4047 which is a modified polyurethane, EFKA PX 4300, EFKA ULTRA PA 4510 and EFKA ULTRA PA 4530 which are modified polyacrylates, EFKA FA 4620 which is an acidic polyether having a viscosity of 1,400 cps, EFKA FA 4642 which is an unsaturated polyamide and acid ester salt having a viscosity of 2,000 cps, HYDROPALAT™ WE 3135, HYDROPALAT WE 3136 and HYDROPALAT WE 3317 which are difunctional block copolymer surfactants terminating in primary hydroxyl groups and having respective viscosities of 375, 450 and 600 cps, and TETRONIC™ 901 and TERTRONIC 904 which are tetrafunctional block copolymers terminating in primary hydroxyl groups and having respective viscosities of 700 and 320 cps); Borchers (e.g., BORCHI™ Gen 0451 which is a polyurethane oligomer having a viscosity of about 30,000 cps, BORCHI Gen 0652 which is an amine neutralized acrylic acid copolymer having a viscosity of about 75-300 cps, and BORCHI Gen 1252 and BORCHI Gen 1253 which are acrylic ester copolymers having respective viscosities of about 1,500-3,500 and 50-300 cps); Byk-Chemie (e.g., BYK™ 156 which is a solution of an ammonium salt of an acrylate copolymer, DISPERBYK™ which is a solution of an alkyl ammonium salt of a low-molecular-weight polycarboxylic acid polymer, DISPERBYK-102 which is an acidic copolymer, DISPERBYK™-145 which is a phosphoric ester salt of a high molecular copolymer with pigment affinic groups and a liquid of unspecified viscosity, DISPERBYK-190 which is a solution of a high molecular weight block copolymer with pigment affinic groups, DISPERBYK-2013 which is a structured copolymer with pigment affinic groups having a viscosity of 8,600 cps, DISPERBYK-2055 which is a copolymer with pigment affinic groups and a liquid of unspecified viscosity, DISPERBYK-2060 which is a solution of a copolymer with pigment affinic groups having a viscosity of 3,600 cps, DISPERBYK-2061 which is a solution of a copolymer with pigment affinic groups having a viscosity of 491 cps, DISPERBYK-2091, DISPERBYK-2200 which is a high molecular weight copolymer with pigment affinic groups sold in solid form as pastilles and BYKJET™-9152 which is a copolymer with pigment affinic groups having a viscosity of 21,600 cps); Clariant (e.g., DISPERSOGEN™ 1728 which is an aqueous solution of a novolac derivative having a viscosity of 4,000 cps, DISPEROGEN 2774 which is a novolac alkoxylate having a viscosity of 4,000 cps, GENAPOL™ X 1003 and GENAPOL X 1005 which are fatty alcohol ethoxylates having respective viscosities of about 400 cps and 1,300 cps, HOST PAL BV concentrate which is a sulfate ester having a viscosity of about 2,700 cps); Cray Valley (e.g., SMA1440H which is an ammonia salt of a styrene maleic anhydride copolymer solution); Dow Chemical Co. (e.g., the TAMOL™ family of dispersants including TAMOL 165A and TAMOL 731A); Elementis (e.g., NUOSPERSE™ FA196 which has a viscosity of 1,200 cps); Lubrizol (e.g., SOLSPERSE™ 27000, SOLSPERSE 28000, SOLSPERSE 32000, SOLSPERSE 39000, SOLSPERSE 64000, SOLSPERSE 65000, SOLSPERSE 66000, SOLSPERSE 71000, SOLSPERSE M387, SOLPLUS™ R700 and SOLPLUS K500); Ethox Chemicals, LLC (e.g., the E-SPERSE™ family of dispersants and ETHOX™ 4658); Evonik (e.g., TEGO™ DISPERS 656, TEGO DISPERS 685, TEGO DISPERS 750 W and TEGO DISPERS 757 W); Rhodia Solvay Group (e.g., ABEX 2514 and ABEX 2525 which are nonionic surfactants, RHODACAL™ IPAM which is isopropyl amine dodecylbenzene sulfonate having a viscosity of 10,000 cps, RHODAFAC™ RS-710 which is a polyoxyethylene tridecyl phosphate ester, and the RHODOLINE™ family of dispersants including RHODOLINE 4170 and RHODOLINE 4188); Sasol Wax GmbH (e.g., ADSPERSE™ 100, ADSPERSE 500 and ADSPERSE 868) and Stepan Company (e.g., G-3300 which is an isopropyl amine salt of an alkyl aryl sulfonate having a viscosity of about 6000 cps, POLYSTEP™ A-15 which is a sodium dodecylbenzene sulfonate having a viscosity of about 85 cps, POLYSTEP B-11 and POLYSTEP B-23 which are ethoxylated ammonium lauryl ether sulfates respectively containing 4 or 12 moles of ethylene oxide and having respective viscosities of 66 and 42 cps, and POLYSTEP B-24 which is sodium lauryl sulfate having a viscosity of 100 cps).

Commercial dispersant compositions and systems of the synthetic kind described above are sold by several companies who manufacture polymer systems. These include:
BASF
   Water-based system
      Dispex® Ultra FA, Dispex® AA, Dispex® CX, Dispex® Ultra PX, Dispex® Ultra PA solvent based system
      Efka® FA, Dispex® Ultra FA, Efka® FA, Efka® PU, Efka® PA, Efka® PX
Clariant
   Dispersogen® 1728, Dispersogen® 2774, Dispersogen® 3169, Dispersogen® AN 100, Dispersogen® AN 200, Dispersogen® ECS, Dispersogen® ECO, Dispersogen® LFS 6, Dispersogen® PCE, Dispersogen® PL 30, Dispersogen® PL 40, Dispersogen® PTS, Dispersogen®, Emulsogen® LCN 217, Emulsogen® TS 200, Dispersogen®, Dispersogen® FN, Dispersogen® FSE, Dispersogen® MT 200, Dispersogen® LFH, Dispersogen® 145, Dispersogen® 4387, Hostapal® BV, Dispersogen® LEC, Dispersogen® PSM, Polyglykol 200 LVC, Polyglykol G500, Polyglykol 300, Polyglykol 400
Lubrizol
   Solsperse™3000, Solsperse™, Solsperse™ 8000, Solsperse™, Solsperse™ 12000S, Solsperse™ 13300, Solsperse™ 13400, Solsperse™ 13500, Solsperse™ 13650, Solsperse™ 13940, Solsperse™ 16000, Solsperse™ 17000, Solsperse™ 17940, Solsperse™ 17000, Solsperse™ 18000, Solsperse™ 19000, Solsperse™ 20000, Solsperse™ 21000, Solsperse™ 22000, Solsperse™ 24000SC, Solsperse™ 26000, Solsperse™ 27000, Solsperse™ 28000, Solsperse™ 32000, Solsperse™ 32500, Solsperse™ 32600, Solsperse™ 33000, Solsperse™ 35000, Solsperse™ 35100, Solsperse™ 35000, Solsperse™ 36000, Solsperse™ 36600, Solsperse™ 37500, Solsperse™ 38500, Solsperse™ 39000, solsperse W100.
Byk
   DISPERBYK-102, DISPERBYK-103, DISPERBYK-106, DISPERBYK-107, DISPERBYK-108, DISPERBYK-109, DISPERBYK-110, DISPERBYK-111, DISPERBYK-115, DISPERBYK-118, DISPERBYK-130, DISPERBYK-140, DISPERBYK-142, DISPERBYK-145, DISPERBYK-161, DISPERBYK-162, DISPERBYK-163, DISPERBYK-164, DISPERBYK-166, DISPERBYK-167, DISPERBYK-168, DISPERBYK-170, DISPERBYK-171, DISPERBYK-174, DISPERBYK-180, DISPERBYK-181, DISPERBYK-182, DISPERBYK-184, DISPERBYK-185, DISPERBYK-187, DISPERBYK-190, DISPERBYK-191, DISPERBYK-192, DISPERBYK-193, DISPERBYK-194 N, DISPERBYK-199, DISPERBYK-2000, DISPERBYK-2001, DISPERBYK-2008, DISPERBYK-2009, DISPERBYK-2010, DISPERBYK-2012, DISPERBYK-2013, DISPERBYK-2015, DISPERBYK-2022, DISPERBYK-2023, DISPERBYK-2025, DISPERBYK-2026, DISPERBYK-2050, DISPERBYK-2055, DISPERBYK-2060, DISPERBYK-2061, DISPERBYK-2062, DISPERBYK-2070, DISPERBYK-2080, DISPERBYK-2081, DISPERBYK-2096, DISPERBYK-2117, DISPERBYK-2118, DISPERBYK-2150, DISPERBYK-2151, DISPERBYK-2152, DISPERBYK-2155, DISPERBYK-2157, DISPERBYK-2158, DISPERBYK-2159, DISPERBYK-2163, DISPERBYK-2164, DISPERBYK-2200, DISPERBYK-2205
DOW
   TAMOL™ 1124; TAMOL™ 1254; TAMOL™ 165A; TAMOL™ 2002; TAMOL™ 2011; TAMOL™ 681; TAMOL™ 731A; TAMOL™ 851; TAMOL™ 901; TAMOL™ 945; TAMOL™ 960; TAMOL™ 963; TAMOL™

Following the foregoing principles and guidelines, the pigment microparticles can be dispersed in the composition with the addition of at least one of a dispersant and a wetting agent. While not wishing to be bound by any specific theory, it is believed that only when the pigments are de-aggregated into their primary particles do they deliver the optimum optical performance. For examples, pigments with a primary particle size of 0.02 micron which provide brilliant bright colors, when present as aggregates of around 0.3 micron provide duller colors.

The dispersant serves to protect the pigment microparticles against agglomeration or flocculation either in the dry state or in the solvent. Dispersants also serve as wetting agents. In this capacity, dispersants as wetting agents can be low or higher molecular weight monomeric surfactants (for example, anionic, cationic or amphoteric surfactants). Dispersants as wetting agents can be higher molecular weight surface-active or pigment particle affinic polymers (for example, polyelectrolyte dispersants such as maleic acid copolymers, and polyurethanes or polyacrylates containing carboxylic acid, amine or isocyanate pigment affinic anchor groups or polyethylene imines) or other type of polyelectrolytes.

Representative wetting agents include those available from a variety of suppliers including Air Products and Chemicals (e.g., CARBOWET™ GA-210 surfactant which has a viscosity of 80 cps, CARBOWET GA-221 surfactant which has a viscosity of 100 cps, DYNOL™ 607 superwetter which has a viscosity of 205 cps and DYNOL 800 superwetter which has a viscosity of 230 cps); Dow Chemical Co. (e.g., CAPSTONE™ fluorosurfactants FS 31, FS 34, FS 35, FS 61 and FS 64); and Stepan Company (e.g., STEPWET™ DOS-70 surfactant which contains 70% active ingredients and has a viscosity of 200 cps, and STEPWET DOS-70EA surfactant which contains 70% active ingredients and has a viscosity of 220 cps).

### F Incorporation of Pigment in Dispersant

The pigments described herein can be chosen and/or modified to be similar enough such that a single dispersant can be used. In other instances, where the pigments are different, but compatible, two or more different dispersants can be used. Because of the extreme small size of the pigment microparticles and their affinity, combination of the pigment microparticles and dispersant to form a substantially homogeneous dispersion that can subsequently be modified and/or diluted as desired is to be accomplished before combination with any or all components of the composition.

The pigment microparticles can be dispersed and stabilized in the medium by one or more dispersants the properties and kinds of which are described above. Exemplary dispersants include non-ionic surfactants moderate weight hydrocarbons such as isododecane. The dispersant can either be added to the medium, or to a precursor medium or can form a coating on the microparticles to facilitate dispersion. It is also possible to provide the microparticles with a coating of a dispersant material and additionally provide a further dispersant to the medium, or to a precursor medium, which is used to form the final medium.

The dispersant, either added to the medium or provided as coating, facilitates wetting of the microparticles, dispersing of the microparticles in the medium, and stabilizing of the microparticles in the medium.

The wetting includes replacing of materials, such as air, adsorbed on the surface of the pigment microparticles and inside of agglomerates of the microparticles by the medium. Typically, a complete wetting of the individual microparticles is desired to singularize the particles and to break off agglomerates formed by microparticles adhering to each other.

After wetting, the microparticles can be subjected to de-aggregate and de-agglomerate step, generally referred to as dispersing step. The dispersing step typically includes the impact of mechanical forces such as shear to singularize the microparticles. In addition to shearing to singularize, the microparticles can be broken into even smaller microparticles using, for example, roller mills, high speed mixers, and bead mills. Usual practice involves substantially homogeneous dispersion of the pigments in dispersant through the use of high shear mixing; for example through use to the appropriate ball mill, ultra-high pressure homogenizer or other system known by those skilled in the art of pigment dispersion.

During wetting and dispersing, the exposed total surface area of the microparticles increases which is wetted by the dispersant. The amount of the dispersant may be gradually increased during dispersing to account for the increased surface area.

The dispersant also functions as de-flocculation agent keeping the dispersed microparticles in a dispersed state and prevent that they flocculate to form loose aggregates. This stabilization is also needed for long term storage purposes. Different type of stabilization such as electrostatic stabilization and steric stabilization are possible, and the type of dispersant is selected in view of the medium and the material of the microparticles.

The dispersant may be added to a dry powder of the pigment particles when the particles are milled to a desired size. During milling, or any other suitable technique to singularize the pigment particles or to break them into smaller part, the dispersant comes in contact with and adheres to the surface of the microparticles. Freshly generated microparticle surface during milling will be coated by the dispersant so that, after milling, the microparticles with a coating formed by the dispersant are provided.

The coating with the dispersant can also be carried out in a liquid carrier medium to which the dispersant is added. The microparticles can also be milled in the liquid carrier.

### G Plasticizer

In general, the glass transition temperature or Tg determines the solid-solid transition of a material such as a polymer from a hard glassy material to a soft rubbery material. If the Tg of a material is too high, and the material is a solid, it will be stiff and inflexible at normal temperatures. The crosslinked polymer network coating should be soft, flexible and unnoticeable to touch and sight yet should not flake, break-up or otherwise release from the keratin fiber, and especially from human hair, when stroked by a hand or brushed with a brush. If the glass transition temperature of the crosslinked polymer network coating is too high for the desired use yet the other properties of the polymer coating are appropriate, such as but not limited to color and wash fastness, one or more plasticizers can be combined with the composition embodiments so as to lower the Tg of the components or the crosslinked network coating and provide the appropriate feel and visual properties to the coating.

The plasticizer can be incorporated directly in the composition or can be applied to the in situ hair before or after the composition. The plasticizer can be chosen from the plasticizers usually used in the field of application.

The plasticizer or plasticizers can have a molecular mass of less than or equal to 5,000 g/mol, such as less than or equal to 2,000 g/mol, for example less than or equal to 1,000 g/mol, such as less than or equal to 900 g/mol. In at least one embodiment, the plasticizer, for example, has a molecular mass of greater than or equal to 40 g/mol.

Thus, the composition can also comprise at least one plasticizer. For example, non-limiting mention can be made, alone or as a mixture, of common plasticizers such as: glycols and derivatives thereof, silicones, silicone polyethers, polyesterpolyols; adipic acid esters (such as diisodecyladipate), trimellitic acid esters, sebacic acid esters, azalaeic acid esters; nonlimiting examples of glycol derivatives are diethylene glycol ethyl ether, diethylene glycol methyl ether, diethylene glycol butyl ether or diethylene glycol hexyl ether, ethylene glycol ethyl ether, ethylene glycol butyl ether, or ethylene glycol hexyl ether; polyethylene glycols, polypropylene glycols, polyethylene glycol-polypropylene glycol copolymers, and mixtures thereof, such as high molecular weight polypropylene glycols, for example having a molecular mass ranging from 500 to 15,000 g/mol, for instance glycol esters; propylene glycol derivatives such as propylene glycol phenyl ether, propylene glycol diacetate, dipropylene glycol ethyl ether, tripropylene glycol methyl ether, diethylene glycol methyl ether, and dipropylene glycol butyl ether. Such compounds are sold by Dow Chemical under the names DOWANOL PPH and DOWANOL DPnB; acid esters, for example esters of carboxylic acids, such as triacids, citrates, phthalates, adipates, carbonates, tartrates, phosphates, and sebacates; esters derived from the reaction of a monocarboxylic acid of formula R₁₁COOH with a diol of formula HOR₁₂OH in which Ru and R12, which can be identical or different, are chosen from a linear, branched or cyclic, saturated, or unsaturated hydrocarbon-based chain containing, for example, from 3 to 15 carbon atoms for example the monoesters resulting from the reaction of isobutyric acid and octanediol such as 2,2,4-trimethyl-1,3-pentanediol, such as the product sold under the reference TEXANOL ESTER ALCOHOL by the company Eastman Chemical; oxyethylenated derivatives, such as oxyethylenated oils, such as plant oils, such as castor oil; mixtures thereof.

Among the esters of tricarboxylic acids mention can be made of the esters of triacids wherein the triacid corresponds to formula

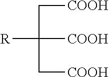

wherein R is a group —H, —OH or —OCOR′ wherein R′ is an alkyl group containing from 1 to 6 carbon atoms. For example, R can be a group —OCOCH₃. The esterifying alcohol for such tricarboxylic acids may be those described above for the monocarboxylic acid esters.

The plasticizer can be present in the composition of the present disclosure in an amount from about 0.01% to 20%.

### H Optional Components

Optional components of the composition include suspending agents, leveling agents and viscosity control agents. The suspending agents help maintain the pigment particles in dispersed condition and minimize or negate their agglomeration. Suspending agents include fatty acid esters of polyols such as polyethylene glycol and polypropylene glycol. These are similar to plasticizers and function in similar fashion to allow pigment particles to “slip” by each other without retarding or binding interaction. They act as grease in this fashion. Additionally, suspending agents in part participate in promoting the stable dispersion of the pigment particles and avoid settling. The at least one bio-based polymer also participates through its solubilization or interaction with the pigment particles and with the medium. The suspending agents provide another factor for maintaining the stable dispersion. They not only provide the “grease” to facilitate Brownian movement but also in part stabilize through interaction as emulsifiers of the pigment particles in the medium. The composition further may contain a catalyst or substance that will lower the reaction activation energy needed for initializing crosslinking.

The composition embodiments in accordance with the present invention can also optionally contain at least one adjuvant, chosen, for example, from reducing agents, fatty substances, softeners, antifoams, moisturizers, UV-screening agents, mineral colloids, peptizers, solubilizers, fragrances, anionic, cationic, nonionic, or amphoteric surfactants, proteins, vitamins, propellants, oxyethylenated or non-oxyethylenated waxes, paraffins, C₁₀-C₃₀ fatty acids such as stearic acid or lauric acid, and C₁₀-C₃₀ fatty amides such as lauric diethanolamide.

The composition embodiments in accordance with the present invention can further optionally contain one or more additives, including, but not limited to, antioxidants (e.g., phenolics, secondary amines, phosphites, thioesters, and combinations thereof), crosslinking agents, reactive diluents (e.g., low molecular weight mono- or di-functional, non-aromatic, (meth)acrylate monomers such as 1,6-hexanediol di(meth)acrylate, tripropylene glycol di(meth)acrylate, isobornyl(meth)acrylate, 2(2-ethoxyethoxy)ethyl(meth)acrylate, n-vinyl formamide, tetrahydrofurfuryl(meth)acrylate, polyethylene glycol di(meth)acrylate, tripropylene glycol di(meth)acrylate, neopentyl glycol dialkoxy di(meth)acrylate, polyethyleneglycol di(meth)acrylate, and mixtures thereof), non-reactive diluents (e.g., ethylene glycol, di(ethylene glycol), tetra(ethylene glycol), glycerol, 1,5-pentanediol, ethylene glycol monomethyl ether, ethylene glycol dimethyl ether, triethylene glycol monomethyl ether, 2-ethoxyethanol, solketal, dyes, fillers (e.g., silica; carbon black; clay; titanium dioxide; silicates of aluminum, magnesium, calcium, sodium, potassium and mixtures thereof; carbonates of calcium, magnesium and mixtures thereof; oxides of silicon, calcium, zinc, iron, titanium, and aluminum; sulfates of calcium, barium, and lead; alumina trihydrate; magnesium hydroxide and mixtures thereof), plasticizers (e.g., petroleum oils such as ASTM D2226 aromatic oils; paraffinic and naphthenic oils; polyalkylbenzene oils; organic acid monoesters such as alkyl and alkoxyalkyl oleates and stearates; organic acid diesters such as dialkyl, dialkoxyalkyl, and alkyl aryl phthalates, terephthalates, sebacates, adipates, and glutarates; glycol diesters such as tri-, tetra-, and polyethylene glycol dialkanoates; trialkyl trimellitates; trialkyl, trialkoxyalkyl, alkyl diaryl, and triaryl phosphates; coumarone-indene resins; pine tars; vegetable oils such as castor, tall, rapeseed, and soybean oils and esters and epoxidized derivatives thereof; esters of dibasic acids (or their anhydrides) with monohydric alcohols such as o-phthalates, adipates and benzoates; and the like and combinations thereof), processing aids, ultraviolet stabilizers (e.g., a hindered amine, an o-hydroxy-phenylbenzotriazole, a 2-hydroxy-4-alkoxybenzophenone, a salicylate, a cyanoacrylate, a nickel chelate, a benzylidene malonate, oxalanilide, and combinations thereof), and combinations thereof.

An additional additive may be a tactile (hair feel) modification agent. These may include, but are not limited to, a softening and/or lubricating and/or anti-static and/or hair alignment and/or anti-frizz benefit and/or impact on the keratin fibers.

An additional additive can be small molecule (non-polymeric) materials which can crosslink the bio-based polymer. Examples of such small molecules are diamines, triamines, tetramines, silanes, and in particular alkoxysilanes, aminoalkoxysilanes, bipodal silanes having nitrogen or sulfur atoms.

### I The Medium

The medium of the composition embodiments of the invention may be water alone, or water in mixture with a volatile polar protic or aprotic organic solvent. In general, the medium is an aqueous solvent suitable for dissolving the at least one bio-based polymer of the embodiments of the composition described herein, for dissolving the crosslinker, if present, and for dispersing the pigment microparticles of the coloring aspects of the compositions described herein. In addition to water present in the medium, a volatile solvent may be present including a volatile polar protic or aprotic organic solvent. Volatile organic solvents of which non-limiting mention may be made include: volatile pyrolidones 1-methylpyrrolidin-2-one, volatile C₁-C₄ alkanols such as methanol, ethanol or isopropanol; esters of liquid C₂C₆ acids and of volatile C₁-C₈ alcohols such as methyl acetate, n-butyl acetate, ethyl acetate, propyl acetate, isopentyl acetate, or ethyl 3-ethoxypropionate; ketones that are liquid at room temperature and volatile, such as methyl ethyl ketone, methyl isobutyl ketone, diisobutyl ketone, isophorone, cyclohexanone, or acetone; volatile polyols such as ethylene glycol and propylene glycol.

According to at least one embodiment of the present disclosure, the organic solvent is chosen from ethanol, isopropanol, acetone, and isododecane.

The medium may be present in the composition according to the present disclosure in an amount ranging from about 0.1% to about 95% by weight, such as from about 1% to about 70% by weight, for example ranging from 5% to 90% by weight relative to the total weight of the composition.

### J Component Concentrations & Physical Parameters

The concentrations of the components of the compositions described herein, and important physical parameters of the compositions are described below.

### J1 The Concentrations of Components

The concentration of each of the at least one bio-based polymer in the composition may range from about 0.25% to about 20%, preferably about 0.5% to about 15%, more preferably about 0.75% to about 10% relative to the total weight of the composition. A preferred concentration of the total of the bio-based polymers in the composition ranges from about 0.5% to about 30%, more preferably about 1.0% to about 25% and most preferably about 1.5% to about 15% by weight relative to the total weight of the composition. The concentration of the total of all polymers (including polymers other than bio-based polymers) in the composition may range from about 0.5 to about 30% by weight relative to the total weight of the composition. A preferred concentration of the total of all polymers (including polymers other than bio-based polymers) in the composition ranges from about 1.0% to about 25%, more preferably about 1.5% to about 20% and most preferably about 1.5% to about 15% by weight relative to the total weight of the composition.

The concentration of crosslinker may vary, based on the type of crosslinker. A preferred concentration of the crosslinker initiating free-radical polymerization may range from about 0.25% to about 20%, preferably about 0.5% to about 15%, more preferably about 0.75% to about 10% relative to the total weight of the composition. A preferred concentration of the crosslinker capable for inducing ionic gelation may range from about 0.25% to about 20%, preferably about 0.5% to about 15%, more preferably about 0.75% to about 10% relative to the total weight of the composition. A preferred concentration of the crosslinker comprising complementary functional groups may range from about 0.25% to about 20%, preferably about 0.5% to about 15%, more preferably about 0.75% to about 10% relative to the total weight of the composition. The amount of the total of all crosslinkers present typically is less than about 30%, preferably less than about 20%, more preferably less than about 15% relative to the total weight of the composition.

The weight ratio of the polymers in the composition to the crosslinkers initiating free-radical polymerization may range from about 10 (1 part polymer:0.1 parts crosslinker) to about 500 (1 part polymer:0.002 parts crosslinker), more preferably from about 20 to about 100. The weight ratio of the polymers in the composition to carbodiimide crosslinkers may range from about 0.2 (1 part polymer:5 parts crosslinker) to about 2.0 (1 part polymer:0.5 parts crosslinker), more preferably from about 0.5 to about 1.0.

The weight ratio of the polymers in the composition to the crosslinkers inducing ionic gelation may range from about 0.2 (1 part polymer:5 parts crosslinker) to about 50 (1 part polymer:0.02 parts crosslinker), more preferably from about 0.85 (1 part polymer:1.2 parts crosslinker) to about 10 (1 part polymer:0.1 parts crosslinker).

The weight ratio of the polymers in the composition to the crosslinkers comprising complementary functional groups may vary depending on the type of polymer(s), and the type of crosslinker used. For modified gelatins using a polythiol crosslinker, for example, the weight ratio of modified gelatin to polythiol may range from about 1 (1 part polymer:1 part crosslinker) to about 20 (1 part polymer:0.05 parts crosslinker), more preferably from about 5 to about 10. For modified gelatins using a polyethylene imine crosslinker, for example, the weight ratio of modified gelatin to polyethylene imine may range from about 1 (1 part polymer:1 part crosslinker) to about 20 (1 part polymer:0.05 parts crosslinker), more preferably from about 5 to about 10. For bio-based polymers using a polyisocyanate crosslinker, for example, the weight ratio of bio-based polymer to polyisocyanate may range from about 0.1 (1 part polymer:10 parts crosslinker) to about 5 (1 part polymer:0.2 parts crosslinker), more preferably from about 0.75 to about 3.5.

### J2 The Viscosity

The viscosity of the composition functions to hold the composition with or without pigment microparticles in place on the keratin fibers while the in situ linked coating is formed. The viscosity substantially avoids free translational flow of the composition. Free translation flow would cause the composition to rapidly run and drip off the surfaces of the hair strands. Nevertheless, the viscosity is not so high that it will not undergo self-leveling to substantially uniformly coat the keratin fibers. Appropriate viscosity of the composition is the result of the interaction of the polymers and crosslinkers present, their concentrations, the pigment microparticles, and as appropriate, an optional viscosity control agent, an optional suspending agent and an optional thickening agent. Generally, the viscosity of the composition may range from about 0.1 to about 200 Pa s⁻¹, preferably 1 to 100 Pa s⁻¹, more preferably 10 to 75 Pa s⁻¹. Viscosity measurements are carried out on a controlled stress rheometer e.g. using an AR2000 type manufactured by TA Instruments, or equivalent instrument. A 6 cm flat acrylic cross hatched parallel plate geometry (TA item 518600.901) and a stainless steel cross hatched base plate (TA item 570011.001) are used. The rheometer is prepared for flow measurements as per standard manufacturer procedure. The parallel plate geometry gap is set to 1000 microns. The flow procedure is programmed to the rheometer with the following conditions: continuous stress ramp 0.1-300 Pa over 2 minutes at 25° C., including 250 measurement points in linear mode. The product is loaded into the geometry as per standard procedure and the measurement commences at 5 min after the mixture preparation. Shear stress value at 10 sec⁻¹ shear rate is obtained from the shear stress vs. shear rate curve, and the corresponding viscosity is calculated by dividing the obtained shear stress by 10.

### J3 The pH

The composition embodiments in accordance with the present disclosure can have a pH ranging from about 3 to about 12, preferably about 4 to about 10 and in many embodiments 6.8 or higher. For example, the pH can be 8 or higher, 9 or higher or at most 12. In some examples, the composition embodiments in accordance with the present invention can have a pH of from about 7 to about 10, about 5 to about 11 or about 6 to about 8.

The pH may range from about 3 to about 8 for polymers that can form cationic groups, e.g., amines and ranging from about 5 to about 11 for polymers that can form anionic groups, e.g., carboxylic and sulfonic acids. For polymers with cation forming groups (amines), preferably the pH is about 4 to about 7 and in many embodiments 6.8 or lower. In some examples, the composition embodiments with polymers having cation forming groups in accordance with the present invention can have a pH of from about 3.0 to about 8.0, preferably about 3.5 to about 6.8, more preferably about 4.5 to about 6.8, most preferably about 5.5 to about 6.5.

The composition in accordance with the present disclosure can comprise a pH modifier and/or buffering agent. The amount is sufficiently effective to adjust the pH of the composition/formulation. Suitable pH modifiers and/or buffering agents for use herein include, but are not limited to: ammonia, alkanolamines such as monoethanolamine, diethanolamine, triethanolamine, monopropanolamine, dipropanolamine, tripropanolamine, tripropanolamine, 2-amino-2-methyl-1-propanol, and 2-amino-2-hydroxymethyl-1,3,-propandiol and guanidium salts, alkali metal and ammonium hydroxides and carbonates, such as sodium hydroxide, sodium silicate, sodium meta silicate and ammonium carbonate, and acids such as inorganic and inorganic acids, e.g., phosphoric acid, acetic acid, ascorbic acid, citric acid or tartaric acid, hydrochloric acid, and mixtures thereof.

### II the Pretreatment Composition

A pretreatment composition can be applied prior to treating keratin fibers with the compositions described herein. A pretreatment composition can be applied to reduce the difference between the root and tip regions of the hair. These two regions have sufficiently different surface properties to make it hard to have a single material which can adhere strongly to both. The pretreatment composition can be applied to all of the hair, or applied to specific sections as needed to cause the desired result of obtaining even color performance and wash resistance root to tip.

The pretreatment composition may also contribute to improved wetting properties of the keratin fibers such as hair for the at least one bio-based polymer. The at least one bio-based polymer can wet the keratin fibers such as hair fibers more quickly and more evenly which contributes to an overall more even coating.

Furthermore, the pretreatment composition also improves the removal properties of the at least one bio-based polymer.

The pretreatment thus can be considered to form a primer coating of the keratin fibers such as surfaces of the hair for improving the wettability, levelling surface properties which would otherwise lead to an uneven wetting, and contributes to the removability of the coating.

### K Aqueous Medium of the Pretreatment Composition

According to embodiments described herein, the aqueous medium of the pretreatment composition comprises water alone, or water in mixture with at least one of polar and protic solvent. In general, the aqueous medium can be any solvent suitable for dissolving the cationic polymer of the embodiments of the pretreatment composition described herein.

### L Cationic Polymer of the Pretreatment Composition

According to embodiments described herein, the pretreatment composition contains a cationic polymer and an aqueous medium. The pretreatment composition is applied prior to the composition of the present invention, either without or with an intermediate rinsing step to optionally remove excess cationic polymer which has not bound to the keratin fibers.

The cationic polymer may be a cationic homopolymer or a cationic heteropolymer. The cationic polymers may be linear or branched.

The cationic polymer may comprise at least one monomer unit, i.e. one or more monomer unit(s), comprising at least one, i.e. one or more, amino functional group(s). The amino functional group(s) may be selected from the group consisting of primary, secondary, tertiary, aromatic amino functional groups and mixtures thereof. Alternatively, the amino functional group may be selected from the group consisting of primary, secondary amino functional groups and mixtures thereof. Alternatively, the amino functional group may be selected from secondary amino functional groups.

According to embodiments described herein, at least 50% of the monomeric units of the cationic polymer contain amino functional group(s), preferably at least 80%, and more preferably at least 90%.

The cationic polymer may have a weight average molecular weight in the range of about 4,000 g/mol to about 450,000 g/mol such as 4,000 g/mol or more to 450,000 g/mol or less, preferably in the range of about 10,000 g/mol to about 200,000 g/mol such as 10,000 g/mol or more to 200,000 g/mol or less, and more preferably in the range of about 10,000 g/mol to about 100,000 g/mol such as 10,000 g/mol or more to 100,000 g/mol or less.

The cationic polymer according to embodiments described herein may be selected from the group consisting of linear polyethyleneimine (linear PEI), branched polyethyleneimine (branched PEI), polyallylamine hydrochloride (PAH), polyallylamine, polyvinylpyridine, and copolymers thereof and mixtures thereof.

According to embodiments described herein, the cationic polymer is selected from linear or branched polyethyleneimines.

Embodiments of the pretreatment composition contain the cationic polymer from 0.1 wt % or more to 5 wt % or less (or 5.0 wt % or less) relative to the total weight of the pretreatment composition, preferably from 0.1 wt % or more to 2 wt % or less (or 2.0 wt % or less) relative to the total weight of the pretreatment composition, more preferably from 0.15 wt % or more to 1.5 wt % or less relative to the total weight of the pretreatment composition, and even more preferably from 0.2 wt % or more to 1 wt % or less (or 1.0 wt % or less) relative to the total weight of the pretreatment composition.

According to embodiments described herein, the cationic polymer is selected from linear or branched polyethyleneimines and contained in the pretreatment composition in the range of from about 0.15 wt % to about 1.5 wt %, such as 0.15 wt % or more to 1.5 wt % or less, and preferably in the range of from about 0.2 wt % to about 1 wt %, such as 0.2 wt % or more to 1 wt % (or 1.0 wt %) or less, relative to the total weight of the pretreatment composition.

According to embodiments described herein, the pretreatment composition can contain one or more cationic polymers. Exemplary cationic polymer having a weight average molecular weight in the range of about 4,000 g/mol to about 450,000 g/mol may be:
a) Linear polyethyleneimine of the formula:

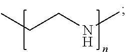

b) Branched polyethyleneimine comprising or consisting of primary, secondary, tertiary amine groups, which may have of the formula:

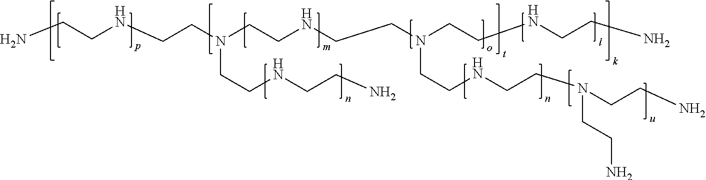

with p, n, m, o, t, l, k, and u being independently selectable, or

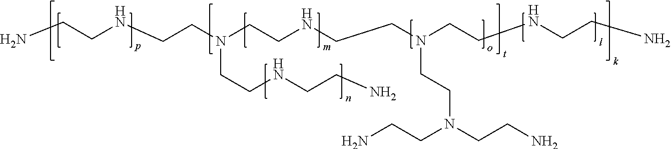

with p, n, m, o, t, l, and k being independently selectable, or

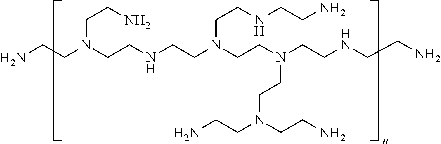

c) Polyallylamine hydrochloride (PAH) of the formula:

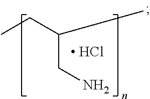

d) Polyallylamine of the formula:

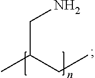

e) polyvinylpyridine of the formula:

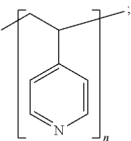

and
g) copolymers thereof and mixtures thereof.

### M the pH of the Pretreatment Composition

According to embodiments described herein, the pH of the pretreatment composition can be in the range between 7 and 10, preferably in the range between 7.4 and 9.6, more preferably in the range between 7.8 and 9.2, and more preferably in the range between 7.0 and 9.0. If the cationic polymer contained in the pretreatment composition is selected from linear or branched polyethyleneimines, the pH of the pretreatment composition preferably will be in the range between 8 and 12, more preferably in the range between 9 and 11, most preferably in the range between 9.5 and 10.5.

Natural hair has a negative surface charge. Without wishing to be tight by theory, it is believed that a higher pH value renders the cationic polymer of the pretreatment composition less charged resulting in an increased loading of the hair surface with the cationic polymer as the cationic polymer is less charged. The thus formed cationic polymeric base layer can thus be provided with a higher polymer loading. This also improves the binding, and loading, of the subsequently formed polymeric coating.

For adjusting the pH, the pretreatment composition can comprise a pH modifier and/or buffering agent such as an alkalizer, an acid, or a combination thereof. PH modifiers and/or buffering agents may be selected from, without being limited to, ammonia, alkanolamines (such as monoethanolamine, diethanolamine, triethanolamine, monopropanolamine, dipropanolamine, tripropanolamine, tripropanolamine, 2-amino-2-methyl-1-propanol, 2-amino-2-hydroxymethyl-1,3,-propandiol), guanidium salts, alkali metal and ammonium hydroxides and carbonates; and mixtures thereof, sodium hydroxide, ammonium carbonate, acidulents (such as inorganic and inorganic acids including for example phosphoric acid, acetic acid, ascorbic acid, citric acid or tartaric acid, hydrochloric acid); and mixtures thereof.

### N Thickening Agent of the Pretreatment Composition

According to embodiments described herein, the pretreatment composition further comprises a thickening agent in the range of 0.1 wt. % to 3 wt. % (or 3.0 wt. %), preferably in the range between 0.3 wt. % to 2.5 wt. %, more preferably 0.5 wt. % to 2.2 wt. %, and even more preferably 0.5 wt. % to 2.0 wt. % relative to the total weight of the pretreatment composition. Preferably, the thickening agent is a non-ionic thickening agent based on polyether-1. A specific example of a polyether-1 based thickening agent is Pure Thix 1442, at a concentration between about 0.5 wt. % and 2.0 wt. %.

### III Application to Keratin Fibers

O Optional Priming and/or Deep Cleaning of Keratin Fibers

A typical procedure for coloration of anagenic hair may involve application of a permanent oxidative dye formulation or may involve semi-permanent application of a direct dye or may involve temporary coloration that can be removed by a single mild shampoo washing. These three techniques for hair coloration traditionally are applied to anagenic hair without a prior wash of the anagenic hair. The presence of sebum, fatty acids (F layer), natural oils, sweat residue, mineral excretion from skin pores are traditionally regarded as helpful in the practice of these coloration techniques. Of course, if the anagenic hair also contains dirt particles, it is usually combed thoroughly to remove dirt debris but the natural oils, secreted minerals, sebum, fatty acids and the like remain.

It is expected therefore that the remanent results demonstrated by formation of a color coating according to the invention on a treated or untreated tress would also be demonstrated by formation on anagenic hair of a color coating according to the invention. In contrast to this expectation, and as shown by the mimic tress experiments described below, a color coating formulated onto anagenic hair such as hair on the scalp of a female hair model does not display long-lasting remanence. The hue, intensity and shade of the color coating on anagenic hair such as that of a model rapidly decreases with each shampooing and by 3 shampoo washes or less, the color coating is gone, especially for color coating on the root portion of anagenic hair.

In contemplation of these results, it was realized that treated and untreated tresses fundamentally differ in at least one respect from anagenic hair. The treated and untreated tresses which are the typical universal substrate for keratin fiber experimentation are not connected to hair follicles and do not receive continuous secretions of sebum, natural oils and fatty acid as well as sweat and mineral secretions from adjacent skin pores. This realization led to experimentation to improve remanence on anagenic hair by converting it to hair like that of treated and untreated tresses. These attempts involved initial removal of sebum, natural oils, fatty acid secretions, sweat and mineral secretions by detergent washing as is usually performed on cut hair being prepared for treated tresses. These attempts also failed. Subsequent body secretions appurtenant to the hair on the scalp of hair models were found to continue circumvention of the remanence result experienced with treated and untreated tresses.

Continued research has led to a combination of aspects that have enabled development of a color coating on anagenic hair that displays remanence similar to that displayed by treated and untreated tresses. These aspects include at least the techniques of Praeparatur and Fundamenta in combination with the Pretreatment Composition with small molecules described above.

### O1 Praeparatur Technique

Substantially complete initial removal of sebum coating the surfaces of anagenic hair delivers a bare hair strand surface exposing the microscopic topographic variability provided by keratin protein at this surface. To obtain such keratin fiber priming, a Praeparatur technique is applied. The Praeparatur technique may be any priming operation that removes sebum from the surfaces of keratin fibers. Exemplary Praeparatur techniques include use of one or more applications of one or more applications of a non-conditioning or substantially non-conditioning surfactant which is free of conditioning actives or substantially free of conditioning additives such as silicones, e.g., amodimethicone, or cetrimonium chloride and polymers such as the polyquaternium versions of cellulose and guar gum derivatives. This technique calls for one or more applications of the surfactant in an aqueous or aqueous-alcoholic medium with optional agents for ionicity and pH control in which the kinds and concentrations of components are adjusted to achieve the desired priming effect. The technique involves use of a mild to moderate aqueous composition of an anionic, non-ionic, amphoteric or zwitterionic surfactant at a concentration beginning at about 2 wt % and escalating to about 30 wt %, preferably up to about 25 wt %, more preferably up to about 10 wt % to about 25 wt % relative to the total weight of the composition. The surfactant composition may also include agents for adjustment of viscosity and ionicity and optional adjustment of pH from acidic to neutral to basic. The surfactant composition may begin with a mild surfactant such as a non-ionic or its mixture with other surfactants and may escalate to higher concentrations of anionic surfactant. A preferable surfactant is an anionic surfactant displaying amphiphilic properties such as an alkali metal salt of a C8-C16 alkyl carboxylate, phosphate, sulfonate, sulfate in which the strength of amphiphilic character increases from carboxylate to sulfate. The initial nonionic surfactant used may be followed by a stronger anionic surfactant and then by a solubilizing anionic surfactant having either a PEG group such as PEG-2 to PEG-20, preferably PEG-2 to PEG-5 for increased hydrophilicity or a PPG group such as PPG-2 to PPG-5 for increased lipophilicity inserted between the anionic head and the alkyl lipophilic tail of the anionic surfactant. Yet stronger solubilizing media may be formulated by increasing the ionic strength and adjusting the pH. Ionicity builders such as alkali metal sulfates, carbonates, phosphates, nitrates and/or xylene sulfonate may be added. The nature of the medium may also be adjusted to provide organic solvents that are capable of solubilizing oils and sebum. Included are C2 to C8 alcohols, preferably isopropanol, isobutanol, tert-butanol and neohexanol. This escalating priming treatment is designed to escalate in mild stepwise fashion so as to avoid overchallenge of the hair.

This escalating priming treatment may be coupled with mechanical agitation such as by a fine tooth comb and/or by a sound vibration such as with a ultrasound device operating at least at 20K Hertz. The mechanical and/or sound vibration can agitate the anagenic hair strands to loosen coatings of sebum, natural oils and secreted sweat and minerals. The ultrasound device may be designed as a fine tooth comb, the teeth of which vibrate to produce the ultrasound. Alternatively, the ultrasound device may be a hand-held generator held in combination with a fine tooth comb which is run through the anagenic hair under the above described priming conditions.

### O2 Fundamenta Technique

The Praeparatur technique often will be sufficient to prime a fiber surface and allow adherence of the pretreatment small molecule with surface exposed protein. However, application of the Praeparatur technique to anagenic hair may not fully prime the bare surface protein of keratin fibers. In such instances, application of the Fundamenta technique may accomplish deep cleaning of the keratin fibers such as anagenic hair. The Fundamenta technique may be applied as a follow-on to use of the Praeparatur technique or applied without prior use of the Praeparatur technique or may be applied first with subsequent use of the Praeparatur technique. The Fundamenta technique structurally deep cleans the surface topography and chemical make-up of the surfaces of keratin fibers and removes the F layer coating on the keratin fibers. The technique also may but not necessarily adjust the topography of the fiber surfaces so as to enable better access of the pretreatment small molecules to the fiber surfaces. This technique may be accomplished by any deep cleaning operation that removes the F layer and deep cleans the keratin fiber surfaces. Exemplary activities include use of one or more of cold plasma discharge, surface oxidizer such as but not limited to ozone, peroxide and/or persulfate and/or a form of chlorite treatment and/or an alkali phase transfer tenside (PETT) such as a multi alkyl ammonium halide, examples of which are C2-C20 alkyl trimethyl ammonium chloride (CTAC) or bromide (CTAB) such as choline halide, cetyl trimethylammonium halide or stearyl trimethylammonium halide.

The cold plasma treatment may be accomplished by passing partially ionized gas over anagenic hair or mimic tresses. Cold plasma is a non-equilibrium atmospheric plasma of a gas such as air or oxygen and/or nitrogen having an effective gas temperature approximating ambient temperature, while the electron temperature may be much higher. The gas is passed between dielectric coated electrodes at a high AC voltage potential difference, or through an RF field. The electromagnetic field dislodges some electrons from the gas atoms to produce a cascade of ionization process which lead to the cold plasma stream. An example is an ozone generator which passes air through a high voltage spark discharge. Cold plasma generators are commercial devices designed for production of ambient temperature (cold) plasma. The plasma is transported through a flexible tube to a nozzle. The nozzle through which the plasma stream flows may be passed over the keratin fibers to accomplish plasma treatment. A typical treatment of a mimic hair tress involve passing the nozzle with flowing plasma over the keratin fibers for approximately 1 to 5 minutes, preferably about 1 to about 3 minutes.

The alkali phase transfer tenside treatment is accomplished by washing anagenic hair and/or mimic tresses with an aqueous solution of phase transfer tenside with an alkaline base or a nucleophile such as an alkoxide. A phase transfer tenside (PETT) generically is a C2-C20 multi-alkyl ammonium halide such as choline or preferably a C12-C20 alkyl trimethyl ammonium chloride or bromide, more preferably cetyl (C16) and/or stearyl (C18) trimethyl ammonium bromide (CTAB). The PETT may be formulated as a 0.1 wt % to 25 wt % aqueous solution. An alkali or thiol aqueous solution (basic alkali pH>10, basic thiol pH>7) of the PETT may be applied to a mimic tress or anagenic hair and massaged through the hair strands either by hand or by brush for a period of 5 to 30 minutes, preferably 5 to 15 minutes to obtain PETT treatment. Thereafter, the tress or anagenic hair, which is substantially saturated with aqueous, basic PETT, is repeatedly rinsed with shampoo in acidic medium to remove the PETT solution.

The surface oxidizer treatment is accomplished by exposing anagenic har or mimic tresses to a dilute oxidizer solution. The oxidizer solution may be formulated as an aqueous solution of a persulfate, hypochlorite, peroxide or ozone typically at a concentration of from about 0.5 wt % to about 10 wt %, preferably about 0.5 wt % to about 5 wt %, more preferably about 0.5 wt % to about 2 wt %. The oxidizer solution can be at an elevated pH, due to the presence of ammonia or MEA or sodium silicate or metasilicate. The oxidizer solution is applied to a mimic tress or anagenic hair and massaged through the hair strands either by hand or by brush for a period of from 10 seconds to about 5 minutes, preferably about 10 seconds to about 1 to 2 minutes. Thereafter the tress or anagenic hair, which is substantially saturated with oxidizer solution, is repeatedly rinsed with water to remove the oxidizer solution.

The basic and/or nucleophilic medium for use with any of the Fundamenta treatment may be any that does not swell the hair strands but will dissolve the resulting free 18-methyleicosanoic acid (F-layer acid). An example of such a medium is t-butoxide in t-butanol or an alkyl or aromatic thiol such as hexyl thiol or thiophenol in acetone.

It is believed that the Praeparatur and Fundamenta techniques enable intimate interaction of the pretreatment small molecule and the surfaces of anagenic hair strands. The Praeparatur and Fundamenta techniques prime and deep clean the hair strand surfaces to remove at least sebum and the F layer so that the small molecule is better able to adhere intimately with and within the peaks, shoulders, and valleys of the surfaces of the keratin fibers as well as with the microscopic topography involving the keratin protein in at the fiber surfaces. This ability of the small molecule is also related to its small size and reactive energy. As mentioned previously, the presence of the sebum and fatty acid f layer surrounding the surfaces of keratin fibers of anagenic hair presents a surprising problem with respect to treated and untreated tresses. The Praeparatur and Fundamenta techniques practiced in combination with the application of the pretreatment composition with small molecules enable this intimate adherence with the microscopic topography of the protein at the surfaces of the keratin fibers. Once in place, the small molecule embodiments are readily able to self-condense to form polysiloxane three dimensional networks in intimate adherence with these keratinaceous surfaces. Subsequent covalent interactions between remaining reactive groups of the condensed small molecule embodiments and the binder component of the film forming composition are believed to extend the three-dimensional network. Because of the microscopic topographic adherence of the three-dimensional network of condensed small molecules to the keratin surface proteins, it is believed that continued sebum and fatty acid secretions onto the keratin fiber shafts are unable to work their way (worm) underneath the network and dislodge it. The network, in turn is intimately interconnected with the three-dimensional network formed through condensation of the binder of the film forming composition. The cooperation of these networks inter-adhered with the keratinaceous surfaces is believed at least in part to provide significant remanence of anagenic hair. These aspects according to the invention are believed to produce at least in part qualities and characteristics of the color coating on the keratinaceous surfaces and especially on the surfaces of hair strands of anagenic hair.

### O3 Application of Praeparatur and Fundamenta Techniques

According to the present invention, one or both of the Praeparatur and Fundamenta techniques may be applied to keratin fibers such as anagen hair. They may be applied separately, applied to different segments of keratin fibers, may be applied sequentially and/or may be applied simultaneously. The Praeparatur technique typically may be applied first, the anagenic hair and the Fundamenta technique may be applied as needed, and especially to the root section of anagenic hair.

The Praeparatur technique typically begins formulation of an aqueous-alcoholic surfactant with the preferred surfactant being an anionic sulfate surfactant. Using high shear mixing techniques and appropriate dilution steps, about 10 to 40 ml of a concentrated anionic surfactant mixture of sodium lauryl sulfate and sodium lauryl ether (PEG₁₀) sulfate may be combined with about 150 to 200 ml of distilled water. A mimic swatch prepared as described in the experimental section may be submersed in the detersive surfactant and briskly agitated with a fine tooth comb for several minutes. If a live salon hair model is the subject of the Praeparatur technique, they may be asked to place her head over a salon wash basin. The salon operator may then first wet the model's hair with water and then apply the surfactant solution to hair and massage and lather the Praeparatur composition onto the hair and scalp. After a period of time the salon operator may then rinse the product from the hair, and optionally repeat the process again. Depending upon the salon operator's or lab technician's visual inspection and touch of the hair, the salon operator/technician may also use a fine toothed comb or pass a hand held ultrasonic device over segments of the hair treated with surfactant solution. The process is continued with optional elevation of the anionic surfactant concentration and optional pH adjustment until the operator/technician's visual inspection and touch of the hair indicates sebum, natural oils, grime and minerals have been removed to expose bare hair shafts.

The Fundamenta technique may be applied separate, alone and independent from the Praeparatur technique or the two may be combined in either order. For a typical combined technique, the Fundamenta technique may be applied following the Praeparatur technique application.

To accomplish the Fundamenta technique, sections of the salon model's hair or sections of the mimic tress may be exposed to a device producing a cold (ambient temperature) plasma, for example a Relyon PZ2 Plasma Pen. A typical cold plasma generator passes a stream of air, nitrogen or oxygen through a high energy RF or EMF field to produce ions and with air and oxygen, also ozone. The stream of partially ionized gas may be directed toward the hair. The result is a “cold plasma” of partially ionized gas on the keratin fibers. The “cold plasma” may be splayed over and through segments of the Praeparatur treated hair to deep clean the surfaces of the hair strands. The cold plasma is applied at a suitable distance over a period of 1 to 5 minutes, preferably 1 to 3 minutes to provide the desired effect of deep cleansing.

In an alternate Fundamenta technique, an aqueous solution of at least 10 wt %, preferably at least 20 wt %, more preferably at least 30 wt % polyalkyl ammonium bromide such as of trimethyl cetyl ammonium bromide (CTAB) or trimethyl stearyl ammonium bromide (STAB) in either alkali at a pH of about 10 or in thiol at a pH above 7 is applied to the mimic hair tress or to sections of a salon model's hair and massaged throughout the tress or hair sections for a period of from about 5 minutes to 30 minutes, preferably 5 minutes to 10 minutes. This treatment is then rinsed with shampoo at acidic pH (with acetic acid) until the CT AB is removed.

In another alternate Fundamenta technique, a composition comprising 1.9 to 12% hydrogen peroxide is mixed with a persulfate bleaching composition which can be a powder. The mixed composition is applied to the hair for a period from about 1 minute to 120 minutes, more preferably from 3 to 40 minutes and then rinsed thoroughly from the hair. In an additional alternate Fundamenta technique, a composition comprising 1.9 to 12% hydrogen peroxide is mixed with a composition contain between 0.1 and 10% of an alkali agent chosen from monoethanolamine or ammonia and ammonium hydroxide. The mixed composition is applied to the hair for a period from about 1 minute to 120 minutes, more preferably from 3 to 40 minutes and then rinsed thoroughly from the hair.

Following practice of either or both of the Praeparatur and Fundamenta technique, the mimic swatch or salon model hair is ready for the Pretreatment step according to the invention.

### O4 Hair Tresses Used for Testing

Three types of hair were used to mimic consumers hair from their tips to their roots. Treated hair tresses. These were tresses which were subjected to the following treatment to reflect consumers tip hair: Natural white undamaged human hair was purchased (Kerling International Haarfabrik GmbH, Backnang, Germany) in the form of 10 cm long and 1 cm wide tresses. The tress was treated with a mixture of Blondor Multi-Blonde bleach powder available from Wella Professionals mixed 1 part with 1.5 parts of 12% Welloxon Perfect available from Wella Professionals. About 4 g of this mixture was applied to each gram of hair. The tresses were then incubated in an oven at 45° C. for 30 minutes after which they were rinsed in water, 37+−3° C. with a flow rate of 4 L/min for 2 minutes and the hair was then dried with a standard Hair dryer from Wella.

Untreated hair tresses. These were used to reflect mid length consumer hair. The Natural white undamaged human hair described above was used as received. Additionally, in some procedure light blonde hair was purchased (Farbe 9/0 from Kerling International Haarfabrik GmbH, Backnang, Germany) in the form of 10 cm long, 1 cm wide strands. The light blonde hair has in prior testing been shown to be a better mimic of consumers root hair, the hair adjacent to the scalp. Whilst not wishing to be bound to theory, it is thought to be less processed by the supplier prior to preparing hair tresses than the natural white hair tresses. These hair tresses were also used as received.
Mimic hair tresses. These are the untreated hair tresses to which synthetic sebum is applied and distributed throughout the hair strands. To simulate the continued sebum secretions of anagenic hair, the synthetic sebum composition is reapplied to the color coated mimic hair tress after each shampoo of the remanent testing
Sebum Insult hair tresses. These are untreated hair tresses which are colored without first receiving a sebum treatment. Following formation of a color coating on the tresses, they are subjected to a sebum coating and dried each time before being subjected to the shampoo cycle.
Untreated hair tresses as purchased and as used are not coated with natural or synthetic sebum but they do have the F layer coating. Mimic hair tresses are coated with synthetic sebum and have the F layer coating as a result of the use of the untreated hair tresses as the starting material for the mimic hair tresses.
The Paeparatur technique removes sebum. The Fundamenta technique removes F layer fatty acid coating on the hair strands. Fundamenta can also remove sebum.

### P Application of the Pretreatment Composition

According to embodiment described herein, the pretreatment composition is applied to keratin fibers such as mammalian hair to impart the surface of the fibers with an overall positive charge which subsequently facilitates adsorption of the at least one bio-based polymer of the composition according to the present invention. The cationic polymer of the pretreatment composition may interact with the surface of the keratin fibers through at least one of electrostatic interaction, van-der-Waals interaction and hydrogen bond interaction, or a combination thereof. The initial interaction may be mediated mainly through electrostatic interaction. Application of the pretreatment composition is optional, however.

Excess liquid of the pretreatment composition may be removed by an absorbing tissue prior to application of the composition according to the present invention, with or without drying the keratin fibers.

After the pretreatment has been accomplished, and the pretreated keratin fibers optionally rinsed, the pretreated keratin fibers can be dried. The keratin fibers can be dried using an elevated temperature. The temperature of the keratin fibers can be increased to elevated temperatures above room temperature such as 40° C. or higher, for example using a hair drier. While the keratin fibers are being dried, some form of interdigitated implement can be used to help separate portions of the keratin fibers, and especially separate hair strands from one another. Examples of interdigitated devices include a comb or a brush. The keratin fibers can be dried with a hair drier while simultaneously being combed or brushed until it is dry to the touch. Alternatively, other means can be employed to dry and separate the keratin fibers such as hair simultaneously. For example, using a combination of air movement and vibrations will accomplish distribution of the composition throughout the strands of hair. Drying the keratin fibers such as hair after application of the pretreatment composition and prior to application of the composition according to the present invention is preferred.

### Q Application of the Composition According to the Present Invention

After the pretreatment has been applied, and optionally rinsed and dried, the composition according to the present invention is applied to the keratin fibers of the hair which have been treated with the pretreatment composition. According to embodiments described herein, the composition according to the present invention is applied without a rinsing step between application of the pretreatment composition and the composition according to the present invention.

The composition according to the present invention may be applied to the keratin fibers in combination with the foregoing pretreatment or may be applied without such pretreatment. Applying the composition according to the present invention—with or without pretreatment—may be repeated one or more times. According to embodiments, the composition according to the present invention may be applied two, three or four times, to create two, three of four layers of the crosslinked polymeric network. According to coloring embodiments, a composition according to the present invention without pigments may be applied one or more times, followed by applying a composition according to the present invention including pigments one or more times, optionally followed by applying a composition according to the present invention without pigments one or more times.

In embodiments where the composition comprises components that might react with each other prematurely (in particular without requiring input of reaction energy such as heat or radiation), such components typically are maintained separately and mixed only shortly prior to application. Application of such reactive components to pretreated or un-pretreated keratin fibers may also be accomplished by sequential application or simultaneous application of these components, in separate media without prior mixing, to the hair. In addition, for the coloring embodiments according to the present disclosure, pigments applied to the hair may be maintained separately and mixed with part or all of the remaining components only shortly prior to application to hair, or applied to the hair sequentially or simultaneously in a separate medium. According to embodiments, the reactive components are maintained separately, and all components including the pigments are mixed shortly prior to application to the hair. According to other embodiments, the composition according to the present invention comprises all required components, and is stable at temperatures below 37° C. with a shelf-life of at least one month, preferably at least three months. According to other embodiments, one or more of the components of the composition according to the present invention may be provided in solid form, for example as lyophilizate, and be reconstituted shortly prior to application to the hair.

For compositions comprising components with complementary functional groups, the rate of reaction of the reactive pairs is pre-adjusted through concentration, steric interaction, temperature, and similar factors controlling reaction rate so that a premix preferably will not substantially interact before the premix is applied to the keratin fibers.

When the composition according to the present invention is applied in combination with a pretreatment, the composition may be applied immediately after pretreatment, or at least 1 hour after pretreatment, or at least 24 hours after pretreatment, or at least 10 days after pretreatment, or at least one month after pretreatment. Typically, the composition will be applied within one hour after application of the pretreatment composition. Preferably, the composition will be applied to keratin fibers at least partially coated with the dried pretreatment composition.

The sequential, simultaneous or premix application of the components according to the present disclosure may be applied to at least a portion of the keratin fibers or may be applied all over the keratin fibers. The components may be applied sequentially, simultaneously or as a premix in a single application over all the keratin fibers or may be applied step-by-step to the keratin fibers. The components may be applied step-by-step, for example, in case the keratin fibers are damaged. Applying the components in a step-by-step manner may help to ensure that the treated portions of the keratin fibers are saturated with the components and may therefore provide a better coverage of the keratin fibers.

The performance of operational method aspects of the present invention can be applied to keratin fibers to form a coating based on the components of the composition, such as a crosslinked polymeric network, optionally including pigment microparticles. This aspect of the invention concerns a method for coloring keratin fibers and comprises applying embodiments of one or more compositions for a time sufficient to deposit an effective colored coating on the keratin fibers or hair strands. A somewhat to substantially overall distribution of the coating on the length and circumference of each fiber is produced.

To accomplish this aspect, embodiments of the composition are applied to the keratin fibers according to the sequences described above by brushing, painting, spraying, atomizing, squeezing, printing, rubbing massaging or in some manner coating the keratin fibers such as hair strands with the embodiments. Following application of a compositional embodiment to the keratin fibers such as hair strands, the composition is set, cured, linked, coordinated and/or otherwise crosslinked for example by increasing the temperature, reducing the water content of the aqueous medium, exposure to electromagnetic radiation, or a combination thereof. Preferably, crosslinking is carried out by warming with blown warm air from a hair dryer or similarly treated to remove the medium, and initiate in situ linking of the at least on bio-based polymer, the crosslinker and the keratin fibers. The setting leaves a substantial to essentially complete overall crosslinked polymeric network of the polymers and crosslinkers containing dispersed pigment microparticles and optional additional components.

The in situ linking of the components during application provides a crosslinked polymeric network or coating that enables it to resist for a time destruction by washing with dilute mixtures of soap and water or shampoo and water. Color fastness (remanence) is developed so that washing with dilute aqueous soap solution or dilute aqueous shampoo will not substantially remove the coating. The properties of the coating include wash-fastness, flexibility, adhesion, abrasion resistance and remanence which are due at least in part to the crosslinked polymeric network and intermolecular entwining, ionic and electrostatic intermolecular interaction, covalent and/or non-covalent linking, dipole interaction and lipophilic interaction of neutral moieties of the compositional constituents.

Selection of the substantive constituents of the composition can be made on the basis of properties such as a solid lattice formation and interaction with the pigment microparticles. Such properties include the flexibility, the hardness, the adhesion, the remanence, the resistance to water or to other chemical compounds, and the abrasion resistance.

The compositions in accordance with the present disclosure can have a viscosity that can be controlled to enable the product to be applied to the hair using either a brush and bowl or a bottle, but with sufficient rheology such that it does not drip and run from the hair onto the face or body. Alternatively, low viscosity formulations may be applied to the hair via a suitable application device such that it does not drip and run form the hair onto the face and body.

The compositions can be utilized in concentrated form or in serial dilutions, to provide for a consistent color results substantially along the entire length of the keratin fibers.

When compositional embodiments of the present invention are applied to hair strands, the treatment may further comprise styling the hair. According to embodiments, styling the hair may comprise perming or straightening the hair. Furthermore, the treatment may comprise conditioning the hair.

The aspect of coloring mammalian or synthetic keratin fibers with a composition as described above includes a method for coloring. The method comprises:
(i) applying the composition according to the present invention to keratin fibers, said composition comprising an effective coloring amount of the at least one bio-based polymer, pigment microparticles, optionally a crosslinker and optional additional components;
(ii) setting the composition by removing or otherwise eliminating the medium (e.g., by drying the composition); and.
(iii) setting the interaction among the complementary functional groups of the components of the composition by initiating the in situ linking among these groups.

During the setting/drying step, color distribution can be facilitated by concurrently moving and/or stroking the hair with an interdigitating device. Interdigitating devices include a comb or brush. The interdigitating device needs to be pulled substantially along the hair strands from root to tip. It can be pulled through at a rate of 0.1 cm s⁻¹ to 50 cm s⁻¹ or at a rate between 0.5 cm s⁻¹ to 20 cm s⁻¹.

The composition is applied to the mammalian or synthetic keratin fibers in any suitable way including spraying the composition, massaging the keratin fibers by hand, after applying the composition to the hand or by combing, brushing or otherwise applying the composition throughout the mammalian or synthetic keratin fibers.

Unlike current hair coloring approaches that use dyes, the color with the compositions described herein occurs on the surface of the hair strands. Current dye based approaches do provide the head of hair with some color variation, as the strands are not identical, and some of these differences are preserved after coloring. There are also differences root to tip which also helps to provide some variation. Using a pigment based surface coloring system such as that of the present invention, the variation of the underlying hair can be substantially removed, leading to a more homogeneous color result. This color result can be a more homogenous application of color. To obtain a somewhat non-homogeneous application of color that tends toward a more natural look, the user can apply the inventive composition by any of several techniques.

The methods by which the compositions described herein are applied can be modified, such that the user applies the product in one region of the hair, and then can apply a diluted version in another region of the hair. The dilution formula is specially chosen to be compatible with the colorant formulation and reduces the coloring strength, while maintaining the longevity of the color result. This can effectively be a “blank” formulation, which contains broadly the same materials as the coloring formulation, but with lower or no pigments present. When diluted the ratio of the diluent to colorant can be between about 10:1 and about 1:10, about 8:1 and about 1:2 or about 5:1 and about 1:1.

Alternatively, the amount of composition applied can be altered in different regions of the hair, for example half the product is applied in the lengths of the hair, leading to a less colorful result. The difference in amounts applied in one region of the hair versus another can be between about 4:1 and about 1:4 or about 2:1 and about 1:2.

Alternatively, a combination of this approaches may be used to deliver the target color variation.

When the foregoing techniques are not possible to be applied, rather than apply a single hair color, it may be possible to apply two or more hair colors to different regions of the hair. When this is done, the different in situ hair colors preferably provide complementary colors so as to develop an attractive result. The difference in colors that can be used, based on the end result on hair tresses such as—natural white hair non pre-bleached are as follows. As described within the CIELCh system:
Color 1 (LCh) versus Color 2 (LCh)
Color 1 L-15<Color 2 L<Color 1 L+15
0 or Color 1 C-10<Color 2 C<Color 1 C+10
Color 1 h-45<Color 2 h<Color 1 h+45

The method for use of the composition in accordance with the present invention can occur during any suitable period. The period of application can be from about 0 to 30 minutes, but in any event a period that is sufficiently long to permit the coating of pigment microparticles to coat and adhere or bind to each separate keratin fiber, substantially along the entire length of each keratin fiber. The resultant is keratin fibers having a color and permanence that is at least equivalent to the color resulting from oxidative in situ hair color, except under much milder conditions.

The compositions described herein can be prepared by the manufacturer as a full shade, e.g., one that is ready to apply to the hair, and then shipped as a discrete unit to the user. The user may need to re-blend the composition prior to application to ensure that the composition delivers the optimum performance. Such re-blending can require shaking the composition for about 1 to about 120 seconds or from about 3 to about 60 seconds. Reblending may also be performed by stirring the composition prior to use. This may occur for about 1 to about 120 seconds or from about 3 to about 60 seconds. Although the compositions according to the present invention are designed to provide stable suspensions of the pigment particles, the re-blending to agitate the microparticles and resuspend them in a substantially uniform distribution is desirable.

Multiple compositions comprising different pigments can be blended together prior to application to the keratin fibers. Such blending can be done in a manner so as to apply a plurality of complementary surface colors to the keratin fibers.

The compositions can include multiple layers, involving multiple applications of at least the first and second components following the first application of the three components. It may be beneficial also to periodically reapply the third component. The techniques for applying multiple layers follow the techniques described above for application of a single composition.

The coating of pigment microparticles comprising at least one pigment in a coating of the substantive constituents of the composition can be adhered to the keratin fibers such as hair utilizing a coating having a total thickness at any given point along the hair fiber of less than about 5 μm, preferably less than about 2 μm as measured using a scanning electron microscope (SEM). To make such measurements, a coated hair sample can be embedded in a suitable resin, and then sectioned root to tip using techniques known to those skilled in the art of scanning electron microscopy. The thickness of the layer on the surface can then be assessed along the line of cuticles over a length of at least 100 μm. The thickness of layer is determined by averaging 10 points evenly spaced over the section of interest.

As described above, application of the composition to sections of keratin fibers such as sections of hair strands can be varied. In addition to varying the concentration of the pigment microparticles and optional coloring agent, different shades and/or colors of composition can be applied to different sections of a strand of hair or a group of strands of hair. For example, the hair roots, mid sections and tips sometimes or often have different shades of color in their natural condition. This variation can be mimicked, altered or covered through use of differing shades or colors of the composition. Roots, for example can be covered with a lighter shade and the tips can be covered with a darker shade to produce a two tone variation of the hair. Application to the hair of a first portion of composition followed by stripping the composition from the hair mid sections and ends followed by setting the remaining composition on the hair roots will provide a first hair color coating on the roots. The mid-sections and tips can be dipped or brush applied with a second portion of composition to complete the two color or two tone treatment. The use of multiple compositions to produce multiple coatings on the hair can provide overlapping, sequential or coterminous coatings on the hair according to typical and routine techniques for applying multiple versions of hair color practiced by professional hair salons.

### R Post Treatment

An optional post treatment composition can be applied after treating the keratin fibers such as hair with the compositions described herein. This can be applied either directly after completion of coloring with the composition. The post treatment can be either single application or multiple application across time. The post treatment can be used to improve one or more of: feel, resistance to shampoo/conditioner/water washing treatments, and shine of the hair. Nonlimiting examples of materials used to improve the feel are those which impart lubricity to the keratin fibers such as hair strands and/or help the hair strands separate during the drying steps. These materials include, for example silicone conditioners, silicone polyethers, silicone polyglucose, polyisobutene, copolymers of ethylene and propylene oxide, and commonly used cosmetic oils and waxes. Nonlimiting examples of materials used to improve shampoo wash resistance are materials which act as a ‘sacrificial layer’ for example polymeric silicones and their copolymers, silicone resins, cosmetics oils and waxes. Nonlimiting examples of materials used to improve the shine of hair (meaning a decrease of the full width at half maximum parameter of the specular reflection curve as measured by a goniophotometer) are those materials which form a smooth film above the previously applied pigment polymer composite on the hair. In general, any cosmetically known film forming material can be used, but preferred are materials such as polymeric silicones and polycationic materials.

### S Remanence and Inspection of Keratin Fibers

Damage caused to the hair by application of the composition and removal of the resulting coating can be assessed by FT-IR (Fourier Transform Infrared) method, which has been established to be suitable for studying the effects on keratin surface damage. (Strassburger, J., J. Soc. Cosmet Chem., 36, 61-74 (1985); Joy, M. & Lewis, D. M., Int. J. Cosmet. Sci., 13, 249-261 (1991); Signori, V. and Lewis, D. M., Int. J. Cosmet. Sci., 19, 1-13 (1997)). In particular, these authors have shown that the method is suitable for quantifying the amount of cysteic acid that is produced from the oxidation of cystine. In general, the oxidation of cystine is thought to be a suitable marker by which to monitor the overall oxidation of the keratinous part of the fiber. Also, the measurement of cysteic acid units by FT-IR is commonly used to study the effects of oxidative treatments or environmental oxidation upon keratin protein containing fibers such as hair and wool.

Signori and Lewis (D. M., Int. J. Cosmet. Sci., 19, 1-13 (1997)) have shown that FT-IR using a diamond Attenuated Total Internal Reflection (ATR) cell is a sensitive and reproducible way of measuring the cysteic acid content of single fibers and bundles. Hence, the method that we have employed to measure the cysteic acid content of multiple fiber bundles and full hair switches, is based upon the FTIR diamond cell ATR method employed by Signori and Lewis (1997). The detailed description of the method used for testing the different damage inhibitors follows thereafter:

A Perkin Elmer Spectrum® 1 Fourier Transform Infrared (FTIR) system equipped with a diamond Attenuated Total Internal Reflection (ATR) cell was used to measure the cysteic acid concentration in mammalian or synthetic hair. In this method, hairswitches of various sizes and colors can be used. The switches were platted (−1 plait per cm) in order to minimize variations in surface area of contact between readings. The Oxidative hair Treatment Protocol described above was repeated for 5 cycles to mimic the behavior of hair after repeated bleaching cycles. Following this treatment, four readings per switch were taken (⅓ and ⅔s down the switch on both sides), and an average calculated. Backgrounds were collected every 4 readings, and an ATR cell pressure of 1 N/m was employed. The cell was cleaned with ethanol between each reading, and a contamination check performed using the monitor ratio mode of the instrument. As prescribed by Signori &amp; Lewis in 1997, a normalized double derivative analysis routine was used. The original spectra were initially converted to absorbance, before being normalized to the 1450 cm⁻¹ band (the characteristic and invariant protein CH₂ stretch). This normalized absorbance was then twice derivatised using a 13 point averaging. The value of the 1450 cm⁻¹ normalized 2nd derivative of the absorbance at 1040 cm⁻¹ was taken as the relative concentration of cysteic acid. This figure was multiplied by −1×10⁻⁴ to recast it into suitable units. It was found that virgin mammalian or synthetic hair produced a value of around 20 cysteic acid units, and heavily oxidized hair produced values of around 170. The following instrumental conditions were employed:
Spectral Resolution—4 cm⁻¹
Data Interval—0.7 cm⁻¹
Mirror Scan Speed—0.2 cm s⁻¹
Number of Background Scans—20
Number of Sample Scans—20
Scan Range—4000 cm⁻¹ to 600 cm⁻¹

When the compositions of the current invention are applied to the hair and then removed, there can be a non-significant change to the level of oxidative damage to the hair, whereas with conventional oxidative colorants there can be a large increase in the measured damage.

The instant disclosure is not limited in scope by the specific compositions and methods described herein, since these embodiments are intended as illustration of several aspects of the disclosure. Any equivalents are intended to be within the scope of this disclosure. Indeed, various modifications in addition to those shown and described herein can be within the grasp of those with ordinary skill in the art. Such modifications are also intended to fall within the scope of the appended claims.

### T Color Selection

Also contemplated herein are compositions having a given color area (gamut principle described above) defined by color coordinates (a*, b*) in the color space represented by the L*a*b* color system, which can be divided into a plurality of color areas. Each of the plurality of colors obtained from the area surrounding a given set of hair fibers is judged to belong to which color area of the colored area of a certain color. The number of colors judged for each color area is counted, and the color of the color area with the largest number of colors is selected as a representative color of the area surrounding a given set of hair fibers.

Also contemplated herein are compositions that do not change the underlying hair color, but instead change some other feature of the hair including shine (e.g., making it shinier or matte), the thickness of the hair and/or the feel of the hair.

When the color is removed from the keratin fibers such as hair, the waste water/composition can be treated to remove the pigments from the waste water effluent system. This can be achieved by filtration, or through cyclone technology, where the density differences are used to force the pigments to the settle, and the water.

### U Testing the Flexibility of a Coating of the Composition

With the crosslinked polymeric network prepared above, testing for optical density to check that the crosslinked polymeric network does not itself alter the hair appearance of the hair too significantly is possible as well.

Further the polymer preferably can have a glass transition point (Tg) as described above so that it is possible to prevent the colored coating of the crosslinked polymeric network from being damaged or cracked and to secure washing and friction fastness.

The coating of the crosslinked polymeric network can have a surface energy between about 20 and about 50 mN m⁻¹. The coating preferably has high transmission, to ensure that it does not interfere with the optics of the hair color. The crosslinked polymeric network preferably has a refractive index between 1.4 and 1.6.

### V Storage Means

The components of the composition may be maintained in separate storage compartments or in separate kit form when the functional groups of these components will react if together, or will catalyze or otherwise cause reaction of such functional groups. A convenient storage means can be utilized such as plastic squeeze tubes, plastic bottles, glass containers, sachets, multi-compartment containers, tooles, spottles syringes and plunger operated dispensing devices. Unit amounts for combination can be formulated so that the entire contents of a unit of one component can be combined with the entire contents of another component for application to the keratin fibers. Alternatively, metered or calibrated dispensing containers for providing measured amounts of the components as directed by printed instructions can be provided. With some embodiments, multiple components can be pre-combined for storage and handling as long as a substantive constituent that would cause in situ linking is maintained in a separate compartment.

Use of the foregoing delivery means enables preparation of an embodiment for practice of the method of the present invention. This embodiment may comprise sequential, simultaneous or premixed application of components of the composition according to the present invention to keratin fibers. Pigment microparticles may be incorporated into one or more components. This aspect of application provides a layer of combined components of the composition according to the present invention on the keratin fibers, which components will undergo transformation to a coating or crosslinked polymeric network, in which functional groups of these components in situ form covalently bonds or interact by hydrogen bonding, electrostatic or ionic interaction, ionic gelation. Preferably the pairs of first and second functional groups are chemically reactive so that covalent bonds are formed between the at least one bio-based polymer, the optional crosslinker, any polymer other than the bio-based polymer(s), if present, and the keratin fibers. With this aspect alone, the resulting coating on sub keratin fibers, such as but not limited to hair, provides good remanence against repeated shampooing, rinsing and contact with mild detergents, soap and similar wash substances.

### W Further Subject Matter

Further subject matter of the present invention is a keratin fiber, comprising a coating of crosslinked bio-based polymers as described herein. The coating is obtainable by the method described above. The keratin fiber preferably is a natural keratin fiber, in particular mammalian hair.

Further subject matter of the present invention is a coating of crosslinked bio-based polymers on a keratin fiber as described herein. The coating is obtainable by the method described above. The keratin fiber preferably is a natural keratin fiber, in particular mammalian hair.

### IV the Kit

The present invention further comprises a kit for treating keratin fibers. The kit may be for treating mammalian keratin fibers such as hair. In particular, the kit may be for treating or coloring human hair.

The kit comprises, in a compartment one or more bio-based polymers. The one or more bio-based polymers may be present in the compartment in the form of an aqueous solution, a semi-solid paste or in solid form, such as a lyophilizate.

The kit may further comprise one or more crosslinkers. The crosslinker(s) may be contained in a separate compartment, different from the compartment containing the one or more bio-based polymers. When present in a separate compartment, the one or more crosslinkers may be present in the compartment in the form of an aqueous solution, a semi-solid paste or in solid form, such as a lyophilizate. When present in the compartment containing the one or more bio-based polymers, the crosslinker(s) are present in the same physical form as the polymer(s).

The kit may further comprise one or more pigments. The pigment(s) may be contained in a separate compartment, different from the compartment containing the one or more bio-based polymers and different from the compartment containing the crosslinker(s), if any. When present in a separate compartment, the one or more crosslinkers may be present in the compartment in the form of an aqueous dispersion, a semi-solid paste or in solid form. When present in the compartment containing another component of the kit, the crosslinker(s) are present in the same physical form as the other component.

According to embodiments, the kit comprises a first compartment containing a lyophilizate of one or more bio-based polymers, a second compartment containing one or more crosslinkers in solid form, a third compartment containing one or more pigments in solid form, and a fourth compartment containing an aqueous medium for reconstituting the composition according to the present invention comprising the bio-based polymer(s), crosslinker(s) and pigment(s). According to embodiments, the kit comprises two or more compartments containing an aqueous medium, for preparing compositions comprising the bio-based polymer(s), crosslinker(s) or pigment(s). Such compositions subsequently may be applied separately to keratin fibers, or two or more of the compositions may be mixed be mixed prior to applying to the keratin fibers such as mammalian keratin fibers, in particular hair.

The kit furthermore may comprise one or more additional components in separate compartments. Additional components comprise for example bleach, shampoo, conditioner, etc.

### EXAMPLES

### General

The coloring compositions described herein within the examples are generally applied to a hair tress with 1.5 mL of composition per approximately 1 g hair tress on a flat plate and brushed into the hair to ensure that the entire strand looks visibly coated. The composition is left to soak in to the hair and afterwards the hair tress is dried for example by heating with a hair dryer while combing until it is dry to the touch.

### 1 Preparation Procedure for Modified Gelatin

### 1.1 Materials

| Chemical | Specification | Supplier |
|---|---|---|
| Gelatin | Gelatin Type B, | Gelita |
| | Limed, Bloom 232 | |
| Methacrylic anhydride | contains 2,000 ppm | Sigma-Aldrich |
| | topanol A as | |
| | inhibitor, 94% | |
| Acrylic anhydride | 95% | ABCR |
| Acetic anhydride | ≥99% | Carl Roth |
| Ethylene diamine | ReagentPlus ®, ≥99% | Sigma-Aldrich |
| Glycidyl acrylate | >95.0%(GC), stabilized | TCI |
| | with MEHQ | |
| N-(3-Dimethylaminopropyl)- | EDC, | Sigma Aldrich |
| N′-ethylcarbodiimide | commercial grade | |
| hydrochloride | | |

### 1.2 Explanation of Abbreviation and Actually Used Modifications

The number behind the letters indicate the molar excess of reactant based on the amino group content of the raw gelatin (amino groups 0.35 mmol/g), except for GME, where the number after E indicates the molar excess based on the carboxy groups. For example in case of GM2A8, 1 g gelatin reacts with 0.7 mmol methacrylic anhydride and 2.8 mmol acetic anhydride, for GAH10 1 g gelatin reacted with 3.5 mmol glycidyl acrylate.

| Gelatin modified with | Abbreviation | Used modifications |
|---|---|---|
| Methacrylic anhydride | GM | GM2, GM10 |
| Acrylic anhydride | GAcry | GAcry10 |
| Glycidyl acrylate | GAH | GAH10, GAH20 |
| Methacrylic anhydride + | GMA | GM2A8, GM5A5 |
| Acetic anhydride | | |
| Methacrylic anhydride + | GME | GM10E10 |
| Ethylene diamine | | |

### 1.3 Modification Process

The following describes the modification of gelatin to GM; the differences in the modification process of the other modified gelatins are listed in the table below.

25 g of gelatin dissolved in 250 mL ultrapure water reacted with a defined molar excess of methacrylic anhydride based on the amount of free amino groups in the gelatin for 5 hours at 37° C. and pH 7.2. The GM then was dialyzed for 4 days, freeze-dried and stored at room temperature until use.

| Modification | Difference in modification process |
|---|---|
| GMA | After 2 h reaction with methacrylic anhydride, |
| | acetic anhydride is added for the |
| | remaining 3 hours reaction time |
| GAcry | Acrylic anhydride is added instead of methacrylic |
| | anhydride, storage of the final |
| | product in freezer |

The reaction conditions for the other modifications of gelatin (other than with anhydride) are listed in the table below.

| Modification | Difference in modification process |
|---|---|
| GAH | 10 w % gelatin dissolved in water reacts |
| | with a desired molar excess of glycidyl |
| | acrylate for 5 hours at 65° C. with the |
| | pH adjusted to 10. |
| | Product is purified by extraction with ethyl |
| | acetate at 40° C. and dialysis for 1 day. |
| | Final product is freeze dried and stored in freezer. |
| GME | 2 w % GM dissolved in 0.1M MES-solution, |
| | pH adjustet to 5, cationized over |
| | night at 37° C. using a desired molar |
| | excess of ethylene diamine based on the |
| | carboxyl groups of the gelatin with the |
| | help of EDC. The product is dialyzed for 4 |
| | days and freeze-dried afterwards. |
| | Storage in a fridge. |

### 2 General Steps Before and After Application of the Composition on the Hair Tress

### 2.1 Hair Preparation

Two types of hair were used: un-damaged and damaged.
Un-damaged hair: Natural white undamaged human hair was purchased from Kerling International Haarfabrik GmbH, Backnang, Germany company in the form of 10 cm long and 1 cm wide strands. This hair was used as received. Natural dark brown, Level 4 hair was purchased from Kerling International Haarfabrik GmbH, Backnang, Germany company in the form of 10 cm long, 1 cm wide strands. This hair was used as received.
Damaged hair which was produced following this procedure: Natural white undamaged human hair was purchased from Kerling International Haarfabrik GmbH, Backnang, Germany company in the form of 10 cm long and 1 cm wide strands and was bleached. The strand was treated with a mixture of Blondor Multi-Blonde bleach powder available from Wella Professionals mixed 1 part with 1.5 parts of 12% Welloxon Perfect available from Wella Professionals. About 4 g of this mixture was applied to each gram of hair. The tresses were then incubated in an oven at 45° C. for 30 minutes after which they are rinsed in water, 37+−2° C. with a flow rate of 4 L/min for 2 minutes and the hair is then dried with a standard Hair dryer from Wella.

### 2.2 Hair Pre-Treatment

Hair prepared as described above is treated with a pre-treatment composition. This composition contains 0.5 wt % of Polyethylenimine (Epomin P1050) in water, the pH is adjusted to 10 if necessary. 1 gram of this composition is applied per 1 gram of hair tress and worked into the hair with fingers to ensure an evenly distribution. The composition is left to soak in for 5 minutes, the excess is wiped of cautiously with a paper towel and the hair tress then is blow dried with a blow dryer while continuously combing.

### 2.3 General Composition Application

To the pre-treated hair tress described as above a freshly prepared coloring composition is added with 1.5 mL per hairtress (hair tress has approximately 1 gram). Application is accomplished by a slow distribution and spreading on the hair tress with a brush. The slow distribution can be accomplished by application with a syringe or a pipette serially to portions of the hair tress. The composition is allowed to soak in by leaving the coated hairtress for different durations (depending on crosslinking system). Excess is removed with absorbent tissue material and the resulting colored hair tress is dried while combing to achieve better hair individualization. Treated hair tresses are kept at rest for at least one night at room temperature.

### 2.4 Standard Wash Procedure

The standard wash procedure is used to determine the lastingness of the colored hair tresses. The hair tresses are left for at least over night at room temperature before hair washes.
1. Rinse the hair tress for approximately 30 seconds with water (4 L min′) at approximately 37+/−2° C.
2. Apply 0.1 g “Wella Professional Brilliance Shampoo for fine and normal hair” without dilution to the individual colored hair tress weighing about 1 g described above.
3. Shampoo is worked into the colored hair tress in the absence of water dilution for 30 sec with fingers by using a stroking motion into the hair.
4. The shampooed colored hair tress is rinsed with water for approximately 30 seconds.
5. The rinsed colored hair tress is then dried using a hot blow dryer while mechanically separating the fibers in the keratin fiber until uniformly dry.
6. Steps 1-5 described above represent one cycle of the standard wash procedure.
7. Repeat of standard wash cycle for multiple cycles and visual comparison of the multiply washed hair tress to an unwashed colored hair tress.

### 3 Preparation and Application of the Different Compositions/Coloring Systems

### 3.1 List of Materials

| Material | Name | Supplier |
|---|---|---|
| PEI | Polyethyleneimine Epomin P-1050 | Nippon Shokubai |
| Isocyanate | BAYHYDUR ultra 304 | Covestro |
| Polythiol | Thiocure ETTMP 1300 | Bruno Bock |
| | | Thiochemicals |
| EDC | N-Ethyl-N′-(3- | Sigma Aldrich |
| | dimethylaminopropyl)carbodiimide | |
| | hydrochloride | |
| CaCl2 | Calcium chloride | AppliChem |
| TPP | Tripolyphosphate | |
| Photoinitiator | Irgacure 2959 | Ciba |
| Photoinitiator | LAP, Lithiumphenyl-2,4,6- | Sigma Aldrich |
| | trimethylbenzoylphosphinate | |
| Pigment | Pigment Red 112, Permanent | Clariant |
| | Red FGR | |
| Pigment | Quindo Magenta 122 | SunChemical |
| Pigment | Pigment Paste 7,5-20% in | Lubrizol |
| Dispersion | Solsperse W100 dispersant | |
| Chitosan | Chitosan 95/200 | Heppe |
| Sodium Alginate | Manucol LKX/Protanal CR8133 | FMC |

### 3.2 Gelatin Crosslinked by UV

### (i) Preparation of Biopolymer Solution

GM2, GM2A8 or GM10 is dissolved in deionized water in a concentration that leads to a final 2 w % or 5 w % in the composition. Dissolving at 37° C. at constant shaking until it is visibly dissolved. 1 w % pigment (in final composition) is added to the solution either as powder and homogenized with the help of an ultrasonic bath, or as an already prepared dispersion.

### (ii) Preparation of Crosslinker Solution

The photoinitiator is dissolved in deionized water at a known weight percentage (for example 0.5 w % or 1 w %) with the help of heat or ultrasonic bath, if needed.

(iii) Preparation of Coloring Composition

A known mass of the previously prepared photoinitiator solution is added to the already prepared gelatin/pigment-solution to have a total of either 1% or 5% photoinitiator based on the mass of modified gelatin. As an example, 200 mg of GM10 in the solution need 2 mg of photoinitiator in case for 1%. The gelatin/pigment/photoinitiator-solution then optionally is mixed for up to half an hour at 37° C. to ensure an even distribution.

### (iv) Crosslinking

The composition is applied to the hair as described above and optionally let rest for a few minutes to soak in. The hairtress then is crosslinked in a chamber equipped with an UVA-lamp with an intensity between 8-20 mW/cm² (depending on the distance of the hair tress to the lamp) while continuously combing to ensure separation of the single hair strands. The chamber is equipped with a small ventilator; the hair tress is crosslinked under UV until dry.

### 3.3 Gelatin Crosslinked by EDC

### (i) Preparation of Biopolymer Solution

Cationized gelatin or unmodified gelatin is dissolved in 0.1 M IVIES-buffer pH 4.8 or 5.5 to lead to a final concentration of 2 w % at 37° C. under constant shaking until visibly dissolved. Pigment is added to the solution to lead to a final concentration of 5 w %.

### (ii) Preparation of Crosslinker Solution

EDC is dissolved in a known concentration in the same IVIES-buffer as the gelatin solution right before usage to avoid hydrolysis.

(iii) Preparation of Coloring Composition

The EDC solution is added to the gelatin/pigment solution to get a final concentration of 0.15 mol/L EDC in the composition. The composition then is added to the hair tress which is not pre-treated with PEI as described above.

### (iv) Crosslinking

The hair is covered and let to rest for 10 or 40 minutes (depending on reactivity). The hair then is dried with a heated blow-dryer while combing until completely dried. 3.4 Sodium alginate crosslinked by calcium chloride

### (i) Preparation of Biopolymer Solution

Sodium alginate is dissolved in deionized water in a concentration of 5 w % at 40-50° C. under constant shaking until visibly dissolved. 1 w % pigment is added to the solution as powder and homogenized by stirring it.

### (ii) Preparation of Crosslinker Solution

Calcium chloride is dissolved in water in a concentration of 10 w % at room temperature under constant stirring.

(iii) Preparation of Coloring Composition

The alginate/pigment-solution is applied to the hair as described above and let rest for 5 minutes to soak into the hair. The hair then is dried with a heated blow-dryer while combing until completely dried.

### (iv) Crosslinking

The dry hair tress coated with sodium alginate and pigment is dipped into the calcium chloride solution for 10 minutes. The hair then is dried with a heated blow-dryer while combing until completely dried.

### 3.5 Chitosan Crosslinked by Tripolyphosphate

### (i) Preparation of Biopolymer Solution

Chitosan-HCl is dissolved in deionized water in a concentration of 5 w % at room temperature under constant shaking until visibly dissolved. 1 w % pigment is added to the solution as powder and homogenized with the help of ultrasonic bath.

### (ii) Preparation of Crosslinker Solution

Tripolyphosphate is dissolved in water in a concentration of 10 w % at room temperature under constant stirring.

(iii) Preparation of Coloring Composition

The chitosan/pigment-solution is applied to the hair as described above and let rest for 5 minutes to soak into the hair. The hair then is dried with a heated blow-dryer while combing until completely dried.

### (iv) Crosslinking

The dry hair tress coated with chitosan and pigment is dipped into the tripolyphosphate solution for 10 minutes. The hair then is dried with a heated blow-dryer while combing until completely dried.

### 3.6 Modified Gelatin Crosslinked by Polythiol

### (i) Preparation of Biopolymer Solution

GAcry10 and GM10 are dissolved in 0.1 M Tris-buffer pH 8.5 or water, GAH10 and GAH20 are dissolved in 0.1 M PBS-buffer pH 7.4 in a concentration that leads to a final concentration of 5 w % in the coloring composition. The modified gelatins are dissolved at 37° C. under constant shaking until visibly dissolved. Pigment is added to lead to a concentration in the final composition of 1 w %.

### (ii) Preparation of Crosslinker Solution

The polythiol is dissolved in the respective buffer in a known concentration with the help of ultrasonic bath.

(iii) Preparation of Coloring Composition

The polythiol is added to the prepared gelatin/pigment solution immediately before use. The polythiol concentration in the final composition is 0.5 w % or 1 w %.

### (iv) Crosslinking

The coloring composition is applied to the hair tress as described above and covered to let it rest at 37° C. for either 15 minutes or optional until the composition visibly gels. The hair then is dried with a heated blow-dryer while combing until completely dried.

### 3.7 Modified Gelatin Crosslinked by Polyethylenimine

### (i) Preparation of Biopolymer Solution

GAcry10 is dissolved in 0.1 M Tris-buffer pH 8.5 at 37° C. under constant shaking until visibly dissolved in a concentration that leads to a final concentration of 5 w %.

### (ii) Preparation of Crosslinker Solution

Polyethylenimine is dissolved in a known concentration in 0.1 M Tris-buffer pH 8.5 with the help of an ultrasonic bath.

(iii) Preparation of Coloring Composition

The polyethylenimine is added to the prepared GAcry10/pigment solution immediately before use to get a final concentration of 0.5 w % in the final composition.

### (iv) Crosslinking

The coloring composition is applied to the hair tress as described above and covered to let it rest at 37° C. for 15 minutes. The hair then is dried with a heated blow-dryer while combing until completely dried.

### 3.8 Gelatin, Sodium Alginate and Chitosan Crosslinked by Isocyanate

### (i) Preparation of Biopolymer Solution

GM2, unmodified gelatin and sodium alginate are dissolved in water, chitosan 95/200 is dissolved in diluted hydrochloric acid with the help of higher temperatures. The concentration of the polymers in the final composition is meant to be 2.9 w %, pigment is added to the dissolved polymer solutions to get a final concentration of 1 w %.

In some examples, 0.01 wt % DABCO is added as a catalyst. From an inspection of the tresses finally obtained, DABCO does not significantly change the result.

### (ii) Preparation of Crosslinker Solution

The polyisocyanate is dissolved or dispersed in a known concentration in the respective diluted hydrochloric acid.

(iii) Preparation of Coloring Composition

Right before the application the polyisocyanate solution is added to the biopolymer/pigment solution to lead to a final concentration of 2.4 w % polyisocyanate.

### (iv) Crosslinking

The composition is added to the hair tress as described above and is covered to react or 15 minutes at 45° C. The hair then is dried with a heated blow-dryer while combing until completely dried.

### 4 Results

### 4.1 Lastingness/Remanence

Remanence was assessed visually by comparing the washed samples versus a retained tress which had been colored but not washed.

The best remanence is obtained by crosslinking chitosan with polyisocyanate. This composition lasted for at least 15 hair washes. The crosslinked GAcry10 also gave quite good results. The poorest lastingnesses were obtained with the modified gelatins that got crosslinked by UV light with the help of a photoinitiator.

### 4.2 Hair Feel

The hairtresses coated with sodium alginate crosslinked with calcium chloride as well as the tresses coated with chitosan crosslinked by TPP felt very brittle. They felt better after a first hairwash, and since nearly nothing was left after 5 washes the tresses felt softer.

The hairtresses coated with gelatin and crosslinked by EDC also felt brittle but not as much as the ones coated with sodium alginate or chitosan.

The tresses coated with gelatin and crosslinked by UV-light/photoinitiator felt greasy which got better after hairwashes.

The hairtresses with gelatin that got crosslinked by polythiol/PEI felt slightly brittle which got better after washing the hair.

For all coating compositions the tresses felt more brittle and sometimes greasy before washing, but it always got better after the first hair wash.

### Biodegradability Testing

### 1 General

The biodegradability of compositions described herein is determined. Bio-based polymers used in the degradation tests comprise bio-based polymers optionally modified olefinoyl-functional groups. The crosslinker used comprises amine-functional groups. The tests determine whether Proteinase K is able to degrade bio-based polymer and crosslinked compositions described herein. Since proteinases are omnipresent in natural environments, the tests described below represent an appropriate simulation of biodegradability.

### 2 Materials

| Chemical | Specification | Supplier |
|---|---|---|
| Gelatin | Gelatin Type B, | Gelita |
| | Limed, Bloom 232 | |
| Proteinase K | P6556 | Sigma-Aldrich |
| Polyethylenimine (PEI) | Epomin P1050/50 | Nippon Shokubai |
| | w % in H₂O | |
| Polyethylene (PE) | Mw 4000, (427772) | Sigma-Aldrich |
| modified gelatin | GAcry10 | in-house |
| PBS | 0.1M PBS (pH 7.4) | in-house |
| PBS//EDTA | 0.01M PBS/0.02% | in-house |
| | EDTA (pH 7.4) | |
| proteinase solution | 10 g/l proteinase | in-house |
| | K in PBS//EDTA | |

### 3 Degradation Tests and Controls

| Sample | | | 0.1M PBS | proteinase | PBS// |
|---|---|---|---|---|---|
| ID# | polymer | crosslinker | buffer | solution | EDTA |
| #1 | 25 mg GAcry10 | 5 mg PEI | 470 μl | 2.8 ml | — |
| #2 | 25 mg GAcry10 | 5 mg PEI | 470 μl | — | 2.8 ml |
| #3 | 25 mg GAcry10 | 5 mg PEI | 470 μL | — | 2.8 ml |
| #4 | 25 mg gelatin | — | 475 μl | 2.8 ml | — |
| #5 | 25 mg gelatin | — | 475 μl | — | 2.8 ml |
| #6 | 25 mg GAcry10 | — | 475 μl | 2.8 ml | — |
| #7 | 25 mg GAcry10 | — | 475 μl | — | 2.8 ml |
| #8 | 25 mg PE | — | 475 μl | 2.8 ml | — |
| #9 | 25 mg PE | — | 475 μl | — | 2.8 ml |

4 Sample preparation

Degradability by Proteinase K is tested for four different materials: unmodified gelatin, GAcry10, GAcry10 crosslinked with PEI, and polyethylene. Therefore, each material was treated with proteinase solution as well as with the PBS//EDTA buffer only (without the Proteinase K) as a control.

To prepare the gels for samples #1 and #2, GAcry10 is dissolved in 0.1 M PBS buffer and PEI is added to the solution. This mixture is gently shaken at 37° C. for 15 minutes for the gel to form. Proteinase solution or PBS//EDTA, respectively, is added to the gel after these 15 minutes.

Sample #3 is a reference example that has the same masses and resulting final concentrations as sample #2, but differs from sample #2 in that GAcry10 is dissolved in both 0.1 M PBS and PBS//EDTA. When PEI subsequently is added to this solution, the concentrations are to low for a gel to form. This liquid GAcry10/PEI solution then can be analyzed and serves as a reference for the degraded GAcry10/PEI gel.

Samples #4-9 are prepared by dissolving the polymer in 0.1 M PBS and subsequently adding the proteinase solution or the PBS//EDTA solution, respectively. The polyethylene does not dissolve in these aqueous solutions and therefore remains as suspension.

### 5 Test Conditions

Degradation tests are carried out at room temperature (22° C.+/−1° C.) under gentle shaking for 2 hours.

The gels with proteinase liquify during the 2 hours degradation time, whereas the gel with buffer only still is intact after 2 hours. For the gel without Proteinase, only the supernatant is analyzed.

### 6 Analytical Method

The samples are analyzed via liquid chromatography-mass spectrometry (LC-MS) using a 1290 Infinity by Agilent and LTQ XL by Thermo Scientific equipped with a PLRP-S column PL1912-3802 by Agilent. The samples are diluted either 1:100 or 1:50, depending on the sample and its resulting intensity in the chromatogram. One measurement takes 45 minutes.
Injection volume: 20.0 μl
Flow: 0.3 ml/min
Column temperature: 22° C.
Mobile Phase: Solvent A: 0.1% Formic acid in water
Solvent B: 0.1% Formic acid in methanol

| Time [min] | B [%] |
|---|---|
| 0 | 10 |
| 30 | 100 |
| 40 | 100 |

In addition to LC-MS chromatography, detection of the samples is carried out with a UV detector at 205 nm. For UV detection, the samples are measured without dilution.

### 7 Test Results

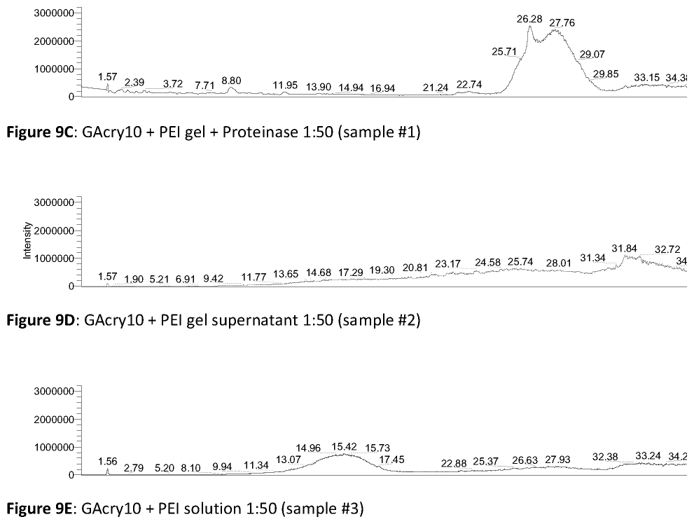

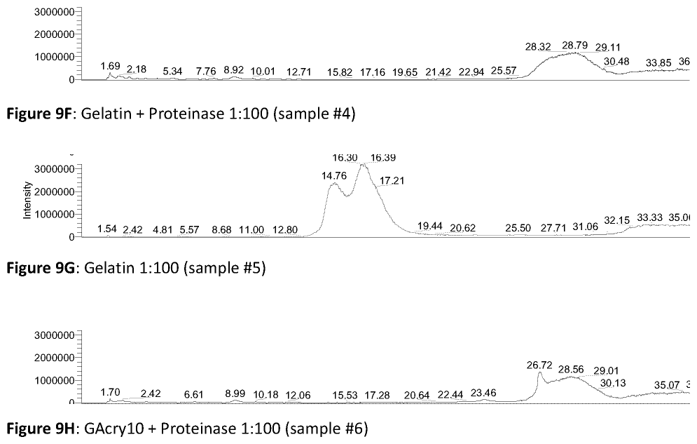

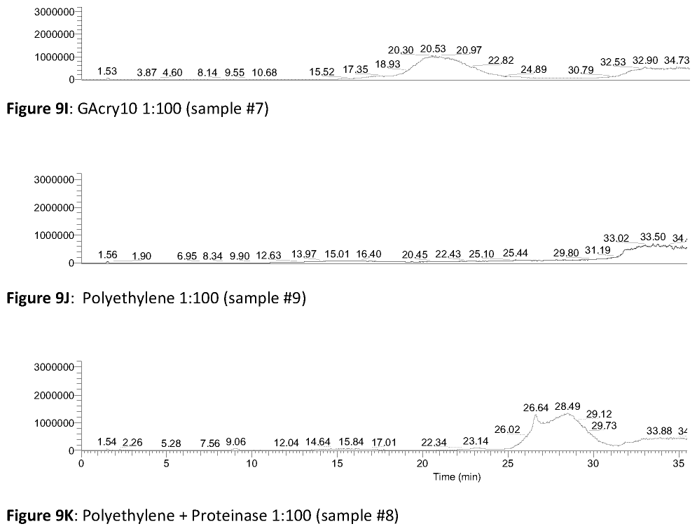

A comparison of the LC-MS chromatograms of Sample IDs #1 and #3 shows that the peak observed for GAcry10 (crosslinked with PEI) disappears completely after degradation treatment with proteinase K (FIGS. 9A and 9E).

Analogously, a comparison of the LC-MS chromatograms of Sample IDs #6-7 shows that the peak observed for GAcry10 (not crosslinked with PEI) disappears completely after degradation treatment with proteinase K (FIGS. 9H and 9I).

A comparison of the LC-MS chromatograms of Sample IDs #4-5 shows that the peak observed for unmodified gelatin disappears completely after degradation treatment with proteinase K (FIGS. 9F and 9G).

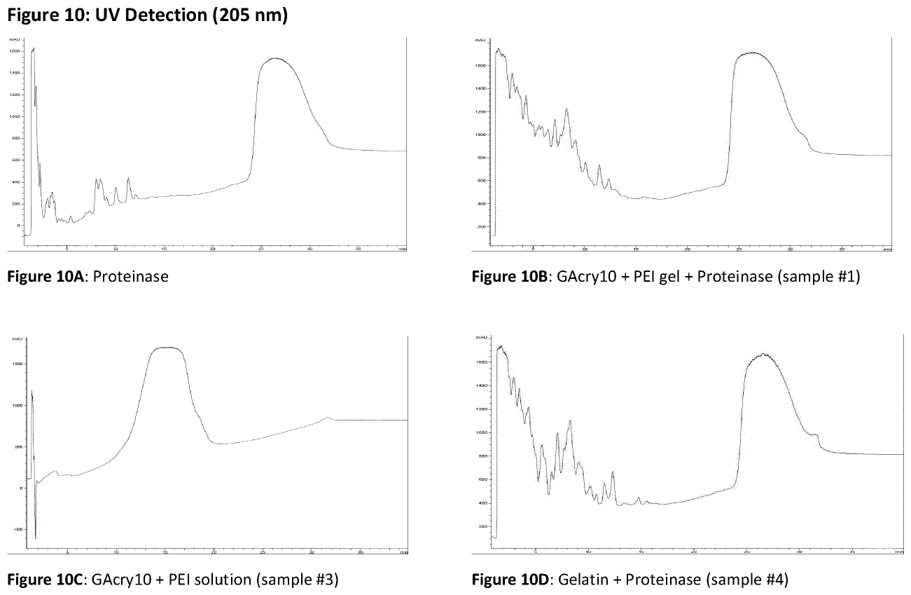

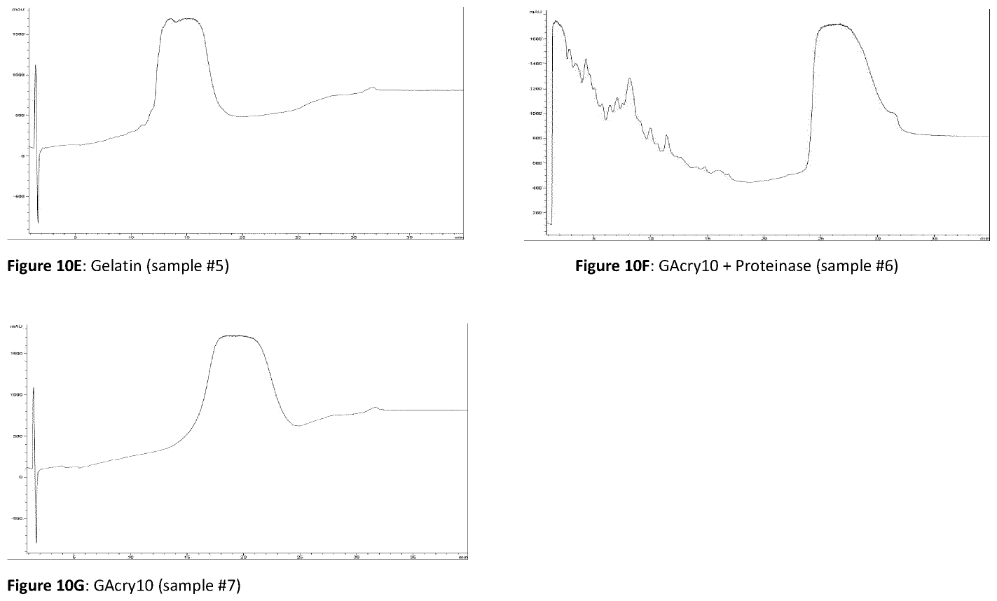

Since no definite peaks for degradation products can be detected in the LC-MS chromatograms of the degraded products (Sample IDs #1, #4 , #6), the undiluted samples are measured by UV detection at 205 nm. In these UV detections, a change in the intensities between 1 min to 15 min can be observed, which most likely can be attributed to degradation products (FIGS. 10A to 10G).

Polyethylene (Sample IDs #8-9) is used as a negative control (FIGS. 9J and 9K). The polyethylene (PE) used is not soluble in the buffer or proteinase-solution used for the other degradation tests. Consequently, no LC-MS chromatogram peak is detected for the PE solution. For PE treated with Proteinase K (Sample ID #9), no additional peaks can be observed as compared to Sample ID #8, which allows for the conclusion that PE is not degraded by Proteinase K in the test format.

The above tests show that bio-based polymers as described herein, bio-based polymers modified with olefinoyl-functional groups as described herein, as well as their crosslinked products with a crosslinker as described herein, are biodegradable.

### SUMMARY STATEMENTS

The inventions, examples and results described and claimed herein may have attributes and embodiments include, but not limited to, those set forth or described or referenced in this application.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, text book or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

The following statements further describe the present invention.
1 A composition for treating a keratin fiber, comprising an aqueous medium and at least one bio-based polymer, or derivative thereof, dissolved in the aqueous medium, wherein the at least one bio-based polymer or derivative thereof comprises first functional groups.
2 The composition of statement 1, wherein the composition comprises less than 20% by weight such as less than 10% by weight, for examples less than 5% by weight of polymers other than bio-based polymers, based on the sum of the dry weight of bio-based polymers and polymers other than bio-based polymers, in particular wherein the composition is essentially free from polymers other than bio-based polymers.
3 The composition of statement 1 or 2, wherein the at least one bio-based polymer is selected from
   a synthetic polymers, wherein the synthetic polymer corresponds to a polymer produced in a cell of a living organism,
   b synthetic polymers produced from renewable resources,
   c synthetic polymers produced from renewable resources, wherein the synthetic polymer corresponds to a polymer produced in a cell of a living organism,
   d biopolymers,
   e combinations thereof.
4 The composition of any of the preceding statements, wherein the at least one bio-based polymer exhibits inherent biodegradability as determined by passing OECD 302C, or exhibits ready biodegradability as determined by passing OECD 301 B, C, D or F, or a combination thereof.
5 The composition of any of the preceding statements, wherein the at least one bio-based polymer is derivatized by:
   a masking of functional groups,
   b demasking of masked functional groups,
   c modifying of functional groups for changing the water solubility,
   d modifying of functional groups for changing the charge density,
   e modifying of functional groups for changing rheological properties,
   f activating of functional groups,
   g combinations thereof
6 The composition of statement 5, wherein the at least one bio-based polymer is derivatized by at least two, in particular by at least three of modifications a-f.
7 The composition of statement 5 or 6, wherein 0.05-60% of the functional groups of the at least one bio-based polymer are modified, in particular 0.01-30%, for example 0.5-10%.
8 The composition of any of the preceding statements, wherein the at least one bio-based polymers is derivatized by
   a hydrolysis,
   b enzymatic treatment,
   c denaturation,
   d combinations thereof.
9 The composition of any of the preceding statements, wherein the at least one bio-based polymer is crosslinkable using electromagnetic radiation or thermal energy.
10 The composition of statement 9, wherein the at least one bio-based polymer crosslinkable using electromagnetic radiation or thermal energy comprises olefinoyl groups.
11 The composition of statement 9 or 10, further comprising a crosslinker, wherein the crosslinker is suitable to induce or promote free-radical polymerization.
12 The composition of any of the preceding statements, further comprising a crosslinker capable of inducing ionic gelation with the at least one bio-based polymer.
13 The composition of statement 12, wherein the crosslinker comprises Ca2+ and the bio-based polymer comprises carboxy-functional groups, in particular wherein the bio-based polymer is an alginate.
14 The composition of statement 12, wherein the crosslinker comprises polyphosphate and the bio-based polymer comprises amine-functional groups, in particular wherein the bio-based polymer is a chitosan.
15 The composition of any of the preceding statements, further comprising a crosslinker comprising second functional groups complementary to the first functional groups of the at least one bio-based polymer.
16 The composition of statement 15, wherein pairs of complementary functional groups are isocyanate and hydroxyl, amine or mercapto or any combination of hydroxyl, amine and mercapto; carboxyl and hydroxyl, amine or mercapto or any combination of hydroxyl, amine and mercapto; alkylepoxy and hydroxyl, amine or mercapto or any combination of hydroxyl, amine and mercapto; olefinoyl and hydroxyl, amine, mercapto, furanyl or pentadienyl or any combination of hydroxyl, amine, mercapto, furanyl or pentadienyl; malonic anhydrido and hydroxyl, amine or mercapto or any combination of hydroxyl, amine and mercapto; formyl and amine or mercapto or any combination of amine and mercapto; vinyl and amine or mercapto or a combination of amine and mercapto; vinyl and furanyl or cyclopentadienyl or a combination of furanyl and cyclopentadienyl or azido and alkynyl.
17 The composition of statement 15 or 16, wherein the functional groups of the crosslinker are selected from isocyanate, mercapto, amino, olefinoyl, or a combination thereof.
18 The composition of any of statements 15-17, wherein pairs of complementary functional groups are isocyanate and hydroxyl, amine or mercapto or any combination of hydroxyl, amine and mercapto.
19 The composition of statement 18, wherein the at least one bio-based polymer is selected from proteins, olefinoyl-modified proteins, polysaccharides, carboxy-functional polysaccharides, amino-functional polysaccharides, olefinoyl-modified polysaccharides, and combinations thereof, and wherein the crosslinker comprises isocyanate-functional groups.
20 The composition of any of statements 15-17, wherein pairs of complementary functional groups are olefinoyl and amine or mercapto.
21 The composition of statement 20, wherein the at least one bio-based polymer comprises olefinoyl-functional groups, and wherein the crosslinker comprises amine-functional groups.
22 The composition of statement 20, wherein the at least one bio-based polymer comprises olefinoyl-functional groups, and wherein the crosslinker comprises mercapto-functional groups.
23 The composition of any of the preceding statements, further comprising a crosslinker, wherein:
   i the at least one bio-based polymer comprises carboxy functional groups and amine functional groups, and/or
   ii the composition comprises at least two bio-based polymers, wherein at least one of the bio-based polymers comprises carboxy functional groups and at least one of the bio-based polymers comprises amine functional groups, and the crosslinker is a carbodiimide.
24 The composition of any of statements 11-23, wherein the at least one bio-based polymer is maintained in a first compartment and the crosslinker is maintained in a second compartment.
25 The composition of any of the preceding statements for coloring a keratin fiber, wherein the composition further comprises one or more pigments dispersed in the aqueous medium.
26 The composition of statement 25, wherein the composition comprises at least one pigment selected from Pigment Yellow 83 (CI 21108, CAS #5567-15-7), Pigment Yellow 155 (C.I. 200310, CAS: 68516-73-4), and Pigment Yellow 180 (C.I. 21290, CAS: 77804-81-0), Pigment Green 36, Pigment Blue 60, Pigment Blue 66, Pigment Blue 16, Pigment Black 6, Pigment White 6, Pigment Red 122, Pigment Red 5, Pigment Red 112, Pigment Violet 19, aluminum flakes, copper flakes, brass flakes.
27 The composition of any of the preceding statements, further comprising one or more of a plasticizer, a dispersant, wetting agent, anti-agglomeration agent, preservative, fragrance, an organic dye compound, a feel-modification agent, or a thickening agent.
28 The composition of any of the preceding statements, wherein the keratin fiber is a natural keratin fiber.
29 The composition of statement 28, wherein the natural keratin fiber is mammalian hair.
30 A method for treating a keratin fiber, comprising applying the composition of any of the preceding statements to a keratin fiber, and promoting crosslinking of the at least one bio-based polymer.
31 The method of statement 30, wherein promoting of crosslinking comprises increasing the temperature, reducing the water content of the aqueous medium, exposure to electromagnetic radiation, or a combination thereof.
32 The method of statement 30 or 31, further comprising, prior to applying the composition, applying a pretreatment composition to the keratin fiber.
33 The method of statement 32, wherein the pretreatment composition comprises a cationic polymer and an aqueous medium.
34 The method of statement 32, wherein the cationic polymer is linear or branched, and comprises one or more amino functional group(s) per polymer chain of the cationic polymer, wherein the amino functional group(s) are selected from primary amino functional groups, secondary amino functional groups, tertiary amino functional groups, aromatic amino functional groups, or combinations thereof.
35 The method of statements 33 or 34, wherein the cationic polymer has weight average molecular weight in the range of about 4,000 g/mol to about 450,000 g/mol.
36 The method of any of statements 33-35, wherein the cationic polymer is in the range from about 0.1 wt % to about 2 wt % relative to the total weight of the pretreatment composition.
37 The method of any of statements 33-36, wherein the cationic polymer is a linear or branched polyethyleneimine.
38 The method of any of statements 32-37, wherein the pretreatment composition has a pH of 7.5 or higher.
39 The method of any of statements 32-38, wherein the pH of the pretreatment composition is between 7.8 and 9.2.
40 The method of any of statements 32-39, further comprising after applying the pretreatment composition and prior to applying the composition, at least partially eliminating the medium of the pretreatment composition from the keratin fiber.
41 The method of any of statements 30-40, further comprising after promoting of crosslinking, rinsing the keratin fiber.
42 The method of any of statements 30-41, further comprising at least partially drying the keratin fiber.
43 The method of any of statements 30-42, wherein the method is for treating hair.
44 The method of statement 43, further comprising styling and/or conditioning the hair.
45 The method of statement 44, wherein styling the hair comprises perming or straightening the hair.
46 The method of any of statements 43-45, further comprising post-treating the hair.
47 Kit for treating hair, comprising in a compartment one or more bio-based polymers.
48 The kit of statement 47, further comprising one or more crosslinkers.
49 The kit of statement 47 or 48, further comprising one or more pigments.
50 Keratin fiber, comprising a coating of crosslinked bio-based polymers obtainable by the method of any of statements 30-46.
51 A coating of crosslinked bio-based polymers obtainable by the method of any of statements 30-46.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims.

1. A composition for coloring a keratin fiber, the composition comprising an aqueous medium, at least one bio-based polymer comprising olefinoyl-functional groups, dissolved in the aqueous medium, at least one crosslinker comprising amine-functional groups and/or mercapto-functional groups, and one or more pigments dispersed in the aqueous medium, wherein the at least one bio-based polymer is selected from proteins and polysaccharides.

2. The composition of claim 1, wherein the composition comprises less than 20% by weight such as less than 10% by weight, for examples less than 5% by weight of polymers other than bio-based polymers, based on the sum of the dry weight of bio-based polymers and polymers other than bio-based polymers, in particular wherein the composition is essentially free from polymers other than bio-based polymers.

3. The composition of claim 1, wherein the at least one bio-based polymer comprises a polymer produced from renewable resources, or a biopolymer.

4. The composition of claim 1, wherein the at least one crosslinker is biodegradable.

5. The composition of claim 1, wherein the at least one bio-based polymer exhibits inherent biodegradability as determined by passing OECD 302C, or exhibits ready biodegradability as determined by passing OECD 301 B, C, D or F, or a combination thereof.

6. The composition of claim 1, wherein the at least one bio-based polymer is selected from collagen, gelatin, keratin, silk fibroin, spider silk, chitosan, and combinations thereof.

7. The composition of claim 1, wherein the olefinoyl-functional groups comprise (meth)acryloyl-functional groups.

8. The composition of claim 1, wherein the crosslinker is selected from polyethyleneimine, polyallylamine, polyvinylamine, aminopolysaccharides, copolymers thereof and mixtures thereof.

9. The composition of claim 1, wherein the crosslinker is a polyethyleneimine.

10. The composition of claim 1, wherein 0.05-60% of the functional groups of the at least one bio-based polymer are olefinoyl-functional groups, in particular 0.1-30%, for example 0.5-10%.

11. The composition of claim 1, wherein the at least one bio-based polymer comprises gelatin, wherein 0.5-15% of the repeating units comprise olefinoyl-functional groups, optionally 0.5-15% of the repeating units comprise masked functional groups, and optionally 0.5-15% of the repeating units comprise cationized functional groups.

12. The composition of claim 1, wherein the one or more pigments are selected from Pigment Yellow 83 (CI 21108, CAS #5567-15-7), Pigment Yellow 155 (C.I. 200310, CAS: 68516-73-4), and Pigment Yellow 180 (C.I. 21290, CAS: 77804-81-0), Pigment Green 36, Pigment Blue 60, Pigment Blue 66, Pigment Blue 16, Pigment Black 6, Pigment White 6, Pigment Red 122, Pigment Red 5, Pigment Red 112, Pigment Violet 19, aluminum flakes, copper flakes, brass flakes, and combinations thereof.

13. The composition of claim 1, wherein the at least one bio-based polymer is maintained in a first compartment and the crosslinker is maintained in a second compartment.

14. The composition of claim 1, further comprising one or more of a plasticizer, a dispersant, wetting agent, anti-agglomeration agent, preservative, fragrance, an organic dye compound, a feel-modification agent, or a thickening agent.

15. The composition of claim 14, wherein the keratin fiber is mammalian hair.

16. A method for coloring a keratin fiber, comprising applying the composition of claim 1 to a keratin fiber, and promoting crosslinking of the at least one bio-based polymer.

17. The method of claim 16, further comprising, prior to applying the composition, applying a pretreatment composition to the keratin fiber.

18. The method of claim 17, wherein the pretreatment composition comprises a cationic polymer and an aqueous medium.

19. The method of claim 18, wherein the cationic polymer is linear or branched, and comprises one or more amino functional group(s) per polymer chain of the cationic polymer, wherein the amino functional group(s) are selected from primary amino functional groups, secondary amino functional groups, tertiary amino functional groups, aromatic amino functional groups, or combinations thereof.

20. The method of claim 18, wherein the cationic polymer has weight average molecular weight in the range of about 4,000 g/mol to about 450,000 g/mol.

21. The method of claim 18, wherein the cationic polymer is in the range from about 0.1 wt % to about 2 wt % relative to the total weight of the pretreatment composition.

22. The method of claim 17, wherein the pretreatment composition has a pH of 7.5 or higher.

23. The method of claim 17, wherein the pH of the pretreatment composition is between 7.8 and 10.5.

24. The method of claim 18, wherein the cationic polymer is a linear or branched polyethyleneimine.

25. The method of claim 24, wherein the pretreatment composition has a pH of 9.5 or higher.

26. The method of claim 17, further comprising after applying the pretreatment composition and prior to applying the composition, at least partially eliminating the medium of the pretreatment composition from the keratin fiber.

27. The method of claim 16, prior to applying the composition and prior to optionally applying the pretreatment composition, further comprising priming the keratin fibers by applying a Praeparatur technique to the fibers, wherein the Praeparatur technique comprises a Praeparatur step of treating the keratin fibers with a substantially non-conditioning or a non-conditioning surfactant composition to produce primed keratin fibers.

28. The method of claim 27, wherein the surfactant composition includes an anionic, nonionic, amphoteric or zwitterionic surfactant or a combination thereof at a concentration of from about 2 wt % to about 30 wt % preferably from about 10 wt % to about 25 wt % relative to the total weight of the composition and optional inclusion of agents for adjustment of viscosity and ionicity and optional adjustment of the pH.

29. The method of claim 16, prior to applying the composition and prior to optionally applying the pretreatment composition, further comprising deep cleaning the keratin fibers by applying a Fundamenta technique to the fibers, wherein the Fundamenta technique comprises a Fundamenta step of deep cleaning the surfaces of the keratin fibers to form deep cleaned keratin fibers.

30. The method of claim 29, wherein the deep cleaning is accomplished by application of at least one of a cold plasma, a phase transfer tenside, an oxidizing agent or a combination thereof to the surfaces of the keratin fibers whereby the deep cleaning at least partially removes F layer fatty acid, sebum and optionally adjusts the topography at the surfaces of the keratin fibers.

31. The method of claim 16, further comprising at least partially drying the keratin fiber.

32. The method of claim 16, wherein the method is for treating hair.

33. The method of claim 27, further comprising post-treating the hair.

34. Kit for coloring hair, comprising one or more bio-based polymers comprising olefinoyl-functional groups, said bio-based polymer(s) selected from proteins and polysaccharides, one or more crosslinkers comprising amine-functional groups and/or mercapto-functional groups, and one or more pigments, wherein the one or more bio-based polymers are contained in a first compartment, the crosslinker(s) optionally are contained in a second compartment different from the first compartment, and the pigment(s) optionally are contained in a compartment different from the first compartment, the second compartment or both.

35. Keratin fiber, comprising a coating of crosslinked bio-based polymers obtainable by the method of claim 16.

36. A coating of crosslinked bio-based polymers obtainable by the method of claim 16.

37. The keratin fiber of claim 35, wherein the coating of crosslinked bio-based polymers is biodegradable.
